(19) United States
(12) Patent Application Publication
(10) Pub. No.: US 2023/0399371 A1
(43) Pub. Date: Dec. 14, 2023

(54) IL-12 COMPOSITIONS AND METHODS OF USE THEREOF

(71) Applicant: Proviva Therapeutics (Hong Kong) Limited, Hong Kong (CN)

(72) Inventors: Zijuan LI, Shanghai (CN); Hongxing ZHOU, Bedford, MA (US)

(21) Appl. No.: 18/034,248

(22) PCT Filed: Oct. 28, 2021
(86) PCT No.: PCT/US2021/057005

§ 371 Date: Apr. 27, 2023

Related U.S. Application Data
(60) Provisional application No. 63/107,023, filed on Oct. 29, 2020.
(60) Provisional application No. 63/147,402, filed on Feb. 9, 2021.

(51) Int. Cl. C07K 14/54; C07K 16/28; A61P 35/00

(52) U.S. Cl. CPC C07K 14/5434; C07K 16/2827; A61P 35/00; C07K 2319/70; C07K 2317/522

(57) ABSTRACT

Provided are attenuated protein homodimers or heterodimers, comprising an immunoglobulin antigen binding domain (ABD), an IL-12A (p35) protein, and an IL-12B (p40) protein, among other optional features, and related pharmaceutical compositions and methods of use thereof.

### CROSS-REFERENCE TO RELATED APPLICATIONS

This application claims the benefit under 35 U.S.C. § 119(e) to U.S. Provisional Application No. 63/107,023, filed Oct. 29, 2020; and U.S. Provisional Application No. 63/147,402, filed Feb. 9, 2021, each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is PRVA_011_02WO_ST25.txt. The text file is about 715 KB, created on Oct. 26, 2021, and is being submitted electronically via EFS-Web.

### BACKGROUND

### Technical Field

The present disclosure relates generally to attenuated protein homodimers or heterodimers, comprising an immunoglobulin antigen binding domain (ABD), an IL-12A (p35) protein, and an IL-12B (p40) protein, among other optional features, and related pharmaceutical compositions and methods of use thereof.

### Description of the Related Art

Interleukin-12 (IL-12) immunotherapy has potential in the treatment of diseases such as cancers and infectious diseases. However, there are certain problems associated with standard IL-12 therapies. For example, IL-12 therapies can have a short half-life in circulation, and have high potency towards a broad range of immune cells. Also, the effects of such IL-therapies are largely systemic, instead of being localized to target tissues, leading to reduced efficacy and increased adverse events (see, for example, Lasek et al., Cancer Immunol Immunother. 63:419-435, 2014).

Nonetheless, IL-12 therapies could be effective, and there is an unmet need in the art to overcome these and other drawbacks. Strategies remain to be developed in order to harness the therapeutic effects of IL-12 for treating patients with cancer, infection and other diseases with high efficacy and low adverse effects (see, for example, Tugues et al., Cell Death and Differentiation, 22: 237-246, 2015).

Embodiments of the present disclosure address these problems and more by providing an attenuated protein homodimers and heterodimers, comprising IL-12 that can be “activated” within or targeted to a disease tissue, for example, a cancer tissue or infected tissue.

### BRIEF SUMMARY

Embodiments of the present disclosure include an attenuated protein homodimer, comprising a first polypeptide and a second polypeptide, wherein:
the first and the second polypeptide comprise, in an N- to C-terminal orientation, a region of an immunoglobulin antigen binding domain (ABD), an IL-12A (p35) protein, a linker, and an IL-12B (p40) protein,
wherein the ABD specifically binds to a cell surface protein expressed on a cell, a plasma protein, or an extracellular matrix (ECM) protein, wherein the IL-12A protein of the first polypeptide is bound to the IL-12B protein of the second polypeptide, and wherein the IL-12B protein of the first polypeptide is bound to the IL-12A protein of the second polypeptide, wherein said binding partially masks a binding site of IL-12 protein(s) that otherwise binds to an IL-12Rβ1/IL-12Rβ2 receptor complex on the surface of an immune cell in vitro or in vivo and attenuates or reduces at least one IL-12 signaling activity of the homodimer relative to wild-type IL-12.

In certain embodiments, the cell surface protein is inducible and co-expressed on an immune cell with the IL-12Rβ1/IL-12Rβ2 receptor complex, or the plasma protein is selected from albumins, globulins, fibrinogens, and clotting factors, or the ECM protein is selected from collagens, elastins, fibronectin, and laminins. In certain embodiments, the cell surface protein is selected from Programmed cell death protein 1 (PD-1), Programmed death-ligand 1 (PD-L1), B7H3 (CD276), T cell immunoreceptor with Ig and ITIM domains (TIGIT), T-cell immunoglobulin and mucin-domain containing-3 (TIM-3), NKG2D, 4-1BB (CD137), CD3, CD4, CD8, CD25, CD70.

In certain embodiments, the ABD comprises a (i) variable heavy chain (VH) region and a CH1 region, and (ii) a variable light chain (VL) region and a CL region, optionally wherein the CH1 region and the CL region are bound together via a disulfide bond. In some embodiments, the C-terminus of the CH1 region is fused to the N-terminus of the p35 protein, optionally via a linker, and wherein the VL/CL region is a separate polypeptide chain that is bound to the VH/CH1 region via the disulfide bond. In certain embodiments, the C-terminus of the CL region is fused to the N-terminus of the p35 protein, optionally via a linker, and wherein the VH/CH1 region is a separate polypeptide chain that is bound to the VL/CL region via the disulfide bond.

In some embodiments, the first and second p35 proteins comprise, consist, or consist essentially of an amino acid sequence that is at least 80, 85, 90, 95, 98, or 100% identical to an amino acid sequence selected from Table S1, optionally wherein the p35 protein is a variant that comprises or retains an amino acid substitution at C74 and/or S197, as defined by the mature p35 sequence, optionally C74A or C74S and/or S197A.

In certain embodiments, the first and second p40 proteins comprise, consist, or consist essentially of an amino acid sequence that is at least 80, 85, 90, 95, 98, or 100% identical to an amino acid sequence selected from Table S2, optionally wherein the p40 protein is a variant that comprises or retains amino acid substitutions at any one or more of Y144, C177, C252, K258, S259, K260, R261, and/or D290, including combinations thereof, as defined by the mature p40 sequence, including any one or more of Y144F, C177A, C252S, K258Q, S259D, K260Q, R261D, and/or D290A, optionally a QDQD substitution at residues K258-R261.

In some embodiments, the linker is a flexible linker, optionally a stable or non-cleavable linker. In some embodiments, the linker is about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length. In some embodiments, the linker is selected from Table L1 or Table L2.

In some embodiments, the immune cell is selected from one or more of a T cell, a B cell, a natural killer (NK) cell, a monocyte, and a macrophage. In some embodiments, the immune cell is an In some embodiments, the first polypeptide and the second polypeptide comprise, consist, or consist essentially of an amino acid sequence that is at least 80, 85, 90, 95, 98, or 100% identical to a sequence selected from Table S3, optionally wherein the attenuated proprotein homodimer comprises chains 3 and 4 selected from Table S3, optionally, wherein the VL/CL region is a separate polypeptide chain that is bound to the VH/CH1 region via the disulfide bond, or wherein the VH/CH1 region is a separate polypeptide chain that is bound to the VL/CL region via the disulfide bond.

In some embodiments, binding of the ABD to the cell surface protein increases binding (avidity) of the IL-12 protein(s) in the homodimer to the IL-12Rβ31/IL-12Rβ2 receptor complex on the surface of an immune cell in vitro or in vivo, and thereby increases at least one IL-12 signaling activity of the homodimer. In some embodiments, binding of the ABD to the cell surface protein increases binding (avidity) of the IL-12 protein(s) in the homodimer to the IL-12Rβ1/IL-12Rβ2 receptor complex by about or at least about 10%, 20%, 30%, 40%, 50%, 60%, 70%, 80%, 90%, 100%, 200%, 300%, 400%, 500%, 1000%, 2000%, 3000%, 4000%, or 5000% or more. In some embodiments, binding of the ABD to the cell surface protein increases at least one IL-12 signaling activity of the homodimer by about or at least about 10%, 20%, 30%, 40%, 50%, 60%, 70%, 80%, 90%, 100%, 200%, 300%, 400%, 500%, 1000%, 2000%, 3000%, 4000%, or 5000% or more. In some embodiments, the at least one IL-12 signaling activity is selected from one or more of stimulating growth and function of T cells, enhancing cytotoxic activity of NK cells and/or CD8+ T cells, stimulating production of interferon-γ (IFN-γ) and/or tumor necrosis factor-α (TNF-α), and inhibiting angiogenesis.

In some embodiments, the attenuated protein homodimer is substantially in homodimeric form in a physiological solution, or under physiological conditions, optionally in vivo conditions.

Certain embodiments include an attenuated protein heterodimer, comprising a first polypeptide and a second polypeptide, wherein:
(a) the first polypeptide comprises, in an N- to C-terminal orientation, a variable heavy chain (VH) region and a CH1 region, an optional linker, and an IL-12A (p35) protein, and the second polypeptide comprises, in an N- to C-terminal orientation, a variable light chain (VL) region and a CL region, an optional linker, and an IL-12B (p40) protein, wherein the p35 protein of the first polypeptide is bound to the p40 protein of the second polypeptide; or
(b) the first polypeptide comprises, in an N- to C-terminal orientation, a variable heavy chain (VH) region and a CH1 region, an optional linker, and an IL-12B (p40) protein, and the second polypeptide comprises, in an N- to C-terminal orientation, a variable light chain (VL) region and a CL region, an optional linker, and an IL-12A (35) protein, wherein the p40 protein of the first polypeptide is bound to the p35 protein of the second polypeptide,
wherein the CH1 region and the CL region of (a) or (b) are bound together via a disulfide bond to form an immunoglobulin antigen binding domain (ABD), wherein the ABD specifically binds to a cell surface protein expressed on a cell, a plasma protein, or an extracellular matrix (ECM) protein, and wherein at least one of the p35 and/or the p40 protein is a variant that has one or more amino acid alterations relative to a wild-type p35 or p40 sequence, and which has reduced binding affinity to a wild-type IL-12Rβ1/IL-12Rβ2 receptor complex on the surface of an immune cell in vitro or in vivo relative to that of the wild-type p35 and/or p40 sequence.

In some embodiments, the cell surface protein is inducible and co-expressed on the immune cell with the IL-12Rβ1/IL-12Rβ2 receptor complex, or the plasma protein is selected from albumins, globulins, fibrinogens, and clotting factors, or the ECM protein is selected from collagens, elastins, fibronectin, and laminins. In some embodiments, the cell surface protein is selected from Programmed cell death protein 1 (PD-1), Programmed death-ligand 1 (PD-L1), B7H3 (CD276), T cell immunoreceptor with Ig and ITIM domains (TIGIT), T-cell immunoglobulin and mucin-domain containing-3 (TIM-3), NKG2D, 4-1BB (CD137), CD3, CD4, CD8, CD25, and CD70.

In some embodiments, the p35 protein is a variant that has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p35 sequence. In some embodiments, the p35 protein has an amino acid substitution at any one or more of Y167, E38, F39, P41, K128, F166, and/or D188, as defined by the mature p35 sequence, optionally selected from any one or more of:
a Y167A, Y167D, Y167E, Y167F, Y167G, Y167H, Y167I, Y167L, Y167N, Y167Q, Y167S, Y167T, or Y167V substitution;
a E38A, E38D, E38F, E38G, E38H, E38I, E38K, E38L, E38M, E38N, E38P, E38Q, E38R, E38S, E38T, E38V, or E38W substitution;
a F39A, F39D, F39E, F39G, F39H, F39I, F39K, F39L, F39M, F39N, F39P, F39Q, F39R, F39S, F39T, F39V, F39W, or F39Y substitution;
a P41A, P41D, P41E, P41F, P41G, P41H, P41I, P41K, P41L, P41M, P41N, P41Q, P41R, P41S, P41T, P41V, P41W, or P41Y substitution;
a K128A, K128D, K128E, K128F, K128G, K128H, K128I, K128L, K128M, K128N, K128P, K128Q, K128R, K128S, K128T, K128V, K128W, or K128Y substitution;
a F166A, F166D, F166E, F166G, F166H, F166I, F166K, F166L, F166M, F166N, F166P, F166Q, F166R, F166S, F166T, F166V, F166W, or F166Y substitution; and
a D188A substitution, including any combination of the foregoing.

In some embodiments, the p35 protein comprises, consists, or consists essentially of an amino acid sequence that is at least 80, 85, 90, 95, 98, or 100% identical to an amino acid sequence selected from Table S1, optionally wherein the p35 protein is a variant that comprises or retains the acid substitution at Y167, and/or comprises or retains an amino acid substitution at C74 and/or S197, as defined by the mature p35 sequence, optionally C74A or C74S and/or S197A.

In some embodiments, the p40 protein is a variant that has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p40 sequence. In specific embodiments, the p40 protein has an amino acid substitution at any one or more of D18, E59, K99, and/or K264, as defined by the mature p40 sequence.

In some embodiments, the p40 protein comprises, consists, or consists essentially of an amino acid sequence that is at least 80, 85, 90, 95, 98, or 100% identical to an amino acid sequence selected from Table S2, optionally wherein the p40 protein is a variant that comprises or retains amino acid substitutions at any one or more of Y144, C177, C252, K258, 5259, K260, R261, and/or D290, including combinations thereof, as defined by the mature p40 sequence, including any one or more of Y144F, C177A, C252S, K258Q, S259D, K260Q, R261D, and/or D290A, optionally a QDQD substitution at residues K258-R261.

In some embodiments, the linker is a flexible linker, optionally a stable or non-cleavable linker. In some embodiments, the linker is about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length. In some embodiments, the linker is selected from Table L1 or Table L2.

Certain attenuated protein heterodimers comprise four polypeptides selected from:
(i) two of the first and second polypeptides of (a); and
(ii) two of the first and second polypeptides of (b),
wherein the four polypeptides are bound together to form an attenuated protein tetramer, for example, wherein the linkers are about or less than about 1, 2, 3, 4, 5, 6, 7, 8, 9, or 10 amino acids in length.

In some embodiments, the immune cell is selected from one or more of a T cell, a B cell, a natural killer (NK) cell, a monocyte, and a macrophage. In some embodiments, the immune cell is an exhausted T cell or an exhausted NK cell. In some embodiments, the first polypeptide comprises, consists, or consists essentially of an amino acid sequence that is at least 80, 85, 90, 95, 98, or 100% identical to a sequence selected from Table S4, wherein the second polypeptide comprises, consists, or consists essentially of an amino acid sequence that is at least 80, 85, 90, 95, 98, or 100% identical to a sequence selected from Table S4.

In some embodiments, binding of the ABD to the cell surface protein increases binding (avidity) of the IL-12 protein(s) in the heterodimer to the IL-12Rβ1/IL-12Rβ2 receptor complex on the surface of the immune cell in vitro or in vivo, and thereby increases at least one IL-12 signaling activity of the heterodimer. In some embodiments, binding of the ABD to the cell surface protein increases binding (avidity) of the IL-12 protein(s) in the heterodimer to the IL-12Rβ31/IL-12Rβ2 receptor complex by about or at least about 10%, 20%, 30%, 40%, 50%, 60%, 70%, 80%, 90%, 100%, 200%, 300%, 400%, 500%, 1000%, 2000%, 3000%, 4000%, or 5000% or more. In some embodiments, binding of the ABD to the cell surface protein increases at least one IL-12 signaling activity of the heterodimer by about or at least about 10%, 20%, 30%, 40%, 50%, 60%, 70%, 80%, 90%, 100%, 200%, 300%, 400%, 500%, 1000%, 2000%, 3000%, 4000%, or 5000% or more. In some embodiments, the at least one IL-12 signaling activity is selected from one or more of stimulating growth and function of T cells, enhancing cytotoxic activity of NK cells and/or CD8+ T cells, stimulating production of interferon-γ (IFN-γ) and/or tumor necrosis factor-α (TNF-α), and inhibiting angiogenesis.

In some embodiments, the attenuated protein heterodimer is substantially in homodimeric form in a physiological solution, or under physiological conditions, optionally in vivo conditions.

Also included are one or more recombinant nucleic acid molecules encoding the first and second polypeptide of an attenuated protein homodimer described herein, and optionally the VL/CL region and/or the VH/CH1 region as a separate polypeptide chain, one or more vectors comprising the recombinant nucleic acid molecules, or one or more host cells comprising the one or more vectors.

Some embodiments include methods of producing an attenuated protein homodimer described herein, comprising culturing the one or more host cells described herein under culture conditions suitable for the expression of the attenuated protein homodimer, and isolating the attenuated protein homodimer from the culture.

Also included are or more recombinant nucleic acid molecules encoding the first and second polypeptide of an attenuated protein heterodimer described herein, one or more vectors comprising the recombinant nucleic acid molecules, or one or more host cells comprising the one or more vectors.

Certain embodiments include methods of producing an attenuated protein heterodimer described herein, comprising
(a) culturing the one or more host cells described herein under culture conditions suitable for the expression of the attenuated protein heterodimer, and isolating the attenuated protein heterodimer from the culture; or
(b) culturing a host cell of described herein that expresses the first polypeptide of the heterodimer, culturing a separate host cell described herein that expresses the second polypeptide of the heterodimer, isolating the first and second polypeptides from each separate host cell, and combining the first and second polypeptides to produce the attenuated protein heterodimer.

Certain embodiments include an isolated human IL-12A (p35) protein variant, which comprises, consists, or consists essentially of an amino acid sequence that is at least 80, 85, 90, 95, 98, or 100% identical to the amino acid sequence set forth in SEQ ID NO: 1 or 2, and which has an amino acid substitution at any one or more of E38, F39, P41, C74, K128, F166, Y167, S197, and/or D188, as defined by the mature p35 sequence. In some embodiments, the isolated human p35 protein variant has an amino acid substitution selected from any one or more of:
a Y167A, Y167D, Y167E, Y167F, Y167G, Y167H, Y167I, Y167L, Y167N, Y167Q, Y167S, Y167T, or Y167V substitution;
a E38A, E38D, E38F, E38G, E38H, E38I, E38K, E38L, E38M, E38N, E38P, E38Q, E38R, E38S, E38T, E38V, or E38W substitution;
a F39A, F39D, F39E, F39G, F39H, F39I, F39K, F39L, F39M, F39N, F39P, F39Q, F39R, F39S, F39T, F39V, F39W, or F39Y substitution;
a P41A, P41D, P41E, P41F, P41G, P41H, P41I, P41K, P41L, P41M, P41N, P41Q, P41R, P41S, P41T, P41V, P41W, or P41Y substitution;
a K128A, K128D, K128E, K128F, K128G, K128H, K128I, K128L, K128M, K128N, K128P, K128Q, K128R, K128S, K128T, K128V, K128W, or K128Y substitution;
a F166A, F166D, F166E, F166G, F166H, F166I, F166K, F166L, F166M, F166N, F166P, F166Q, F166R, F166S, F166T, F166V, F166W, or F166Y substitution; and
a D188A substitution, including any combination of the foregoing. In some embodiments, the isolated p35 protein variant has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p35 sequence.

Certain embodiments include an isolated recombinant nucleic acid molecule encoding a human IL-12A (p35) protein variant described herein, a vector comprising the recombinant nucleic acid molecule, a host cell comprising the vector. Also included are methods of producing the human IL-12A (p35) protein variant described herein, comprising culturing the host cell under culture conditions suitable for the expression of the p35 protein variant, and isolating the p35 protein variant from the culture.

Particular embodiments include pharmaceutical compositions, comprising a pharmaceutically acceptable carrier and an attenuated protein homodimer described herein, an attenuated protein heterodimer described herein, an IL-12A (p35) protein variant described herein, or any combination thereof.

Also included are methods of treating disease in a subject, and/or a method of enhancing an immune response in a subject, comprising administering to the subject a therapeutically effective amount of the pharmaceutical composition described herein. In some embodiments, the disease is selected from one or more of a cancer, a viral infection, and an immune disorder.

In some embodiments, the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, following administration, the attenuated protein homodimer or heterodimer is activated through binding of the ABD to the cell surface receptor on an immune cell in vivo, optionally in a tumor microenvironment, which increases binding (avidity) of the IL-12 protein(s) to the IL-12Rβ1/IL-12Rβ2 receptor complex on the surface of the immune cell, and thereby increases at least one IL-12 signaling activity. In some embodiments, the immune cell is selected from one or more of a T cell, a B cell, a natural killer cell, a monocyte, and a macrophage. In some embodiments, the immune cell is an exhausted T cell or an exhausted NK cell.

In some embodiments, administration of the attenuated protein homodimer or heterodimer increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response. In some embodiments, administration of the attenuated protein homodimer or heterodimer increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

In some embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis, optionally wherein the subject is HIV-positive.

In some embodiments, the immune disorder is selected from one or more of type 1 diabetes, vasculitis, and an immunodeficiency.

In some embodiments, the pharmaceutical composition is administered to the subject by parenteral administration. In specific embodiments, the parenteral administration is intravenous administration.

Also included is the use of a pharmaceutical composition described herein in the preparation of a medicament for treating a disease in a subject, and/or for enhancing an immune response in a subject. Particular embodiments include a pharmaceutical composition described herein for use in treating a disease in a subject, and/or for enhancing an immune response in a subject.

### BRIEF DESCRIPTION OF THE DRAWINGS

FIG. 1A is a ribbon presentation of a disulfide bounded heterodimeric human IL-12 structure (PDB: 1F45) through the binding of p35 and p40, and FIG. 1B is a schematic diagram of the p35 and p40 chains of IL-12.

FIG. 2 is a schematic diagram of wild-type IL-12 that is bound to IL-12 co-receptors on a cell surface. The IL-12 co-receptor is composed of IL-12Rβ1 and IL-12Rβ2 chains and signals through Jak2 and Tyk2, respectively, leading to phosphorylation of STAT4 and other STAT transcription factors.

FIG. 3 is a schematic diagram of an exemplary low-affinity IL-12 variant that is bound weakly to IL-12 co-receptors on a cell surface. The IL-12 variant (mutated at p35, p40, or both) has reduced binding affinity to IL-12 co-receptors, resulting in low or no signaling in the cells.

FIG. 4A is a schematic diagram of an antigen binding domain (ABD) fused to the exemplary low-affinity IL-12 variant of FIG. 3, illustrating the general principle that binding of the ABD to a different cell surface receptor on an IL-12 co-receptor-expressing cell limits the diffusive property of the low-affinity IL-12 variant, resulting in increased binding (avidity) to the IL-12 co-receptors and increased IL-12 signaling activity. FIG. 4B is a schematic diagram of an ABD fused to the exemplary low-affinity IL-12 variant of FIG. 3, illustrating the general principle that binding of the ABD to a non-IL-12 effector cell results in the presentation of multiple copies of IL-12 variants to an effector cells expressing IL-12 co-receptors. This mode of action increases binding of IL-12 variants to the IL-12 co-receptors by increasing binding avidity, and thereby increasing the IL-12 variant signaling activity.

FIG. 5A is a schematic of an exemplary attenuated protein homodimer, as described herein. The homodimeric binding between the p35 and p40 proteins on each separate chain attenuate the binding affinity of the homodimer to IL-12 co-receptors, relative to wild-type IL-12, and binding of the ABD to a cell surface receptor on an immune cell of interest results in increased binding (avidity) of the homodimer to the IL-12 co-receptors and increased IL-12 signaling activity. FIGS. 5B-5C are schematics of exemplary attenuated protein heterodimers, as described herein, which are composed of ABD regions fused to an exemplary low-affinity IL-12 variant. In FIG. 5B, the VH/CH1 regions of the ABD are fused to a P35 variant, the VL/CL regions are fused to a p40 variant, and the CH1/CL regions are bound by a disulfide bond. In FIG. 5C, the VL/CL regions of the ABD are fused to a P35 variant, the VH/CH1 regions are fused to a p40 variant, and the CH1/CL regions are bound by a disulfide bond. As above, binding of the ABD to a cell surface receptor on an immune cell of interest results in increased binding (avidity) of the heterodimer to the IL-12 co-receptors and increased IL-12 signaling activity. FIG. 5D and FIG. 5E are schematics of exemplary tetramers of the heterodimeric structures from FIG. 5B and FIG. 5C, respectively.

FIGS. 6A-6J show representative SDS-PAGE results of purified proteins. FIGS. 6A, 6C, 6E, 6G, and 6I show non-reducing SDS-PAGE results. FIGS. 6B, 6D, 6F, 6H, and 6J show reducing SDS-PAGE results. “M” on the figures represents the protein standard marker.

FIGS. 7A-7L show representative HPLC analysis results of purified proteins.

FIGS. 8A-8C show ELISA binding results of purified proteins to PD-L1 and/or B7H3.

FIGS. 9A-9S show the activity of exemplary IL-12-based protein homodimers and heterodimers relative to wild-type human IL-12, as measured by phosphorylation of STAT4 in preactivated human PBMCs.

FIGS. 10A-10D show the activity of exemplary IL-12-based protein homodimers and heterodimers relative to wild-type human IL-12, as measured by IFN-γ release in preactivated human PBMCs.

FIGS. 11A-11D show the activity of exemplary IL-12-based protein heterodimers relative to wild-type human IL-12, as measured by IFN-γ release in the NK92MI cell line.

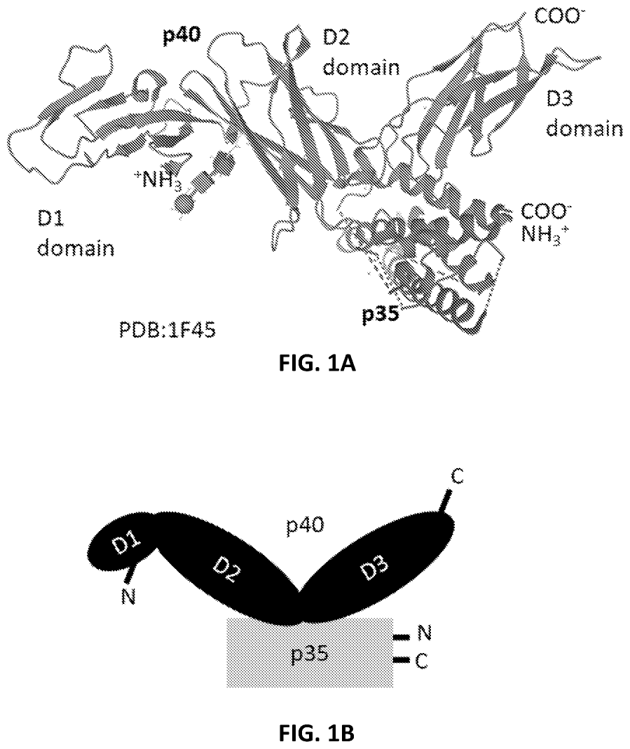

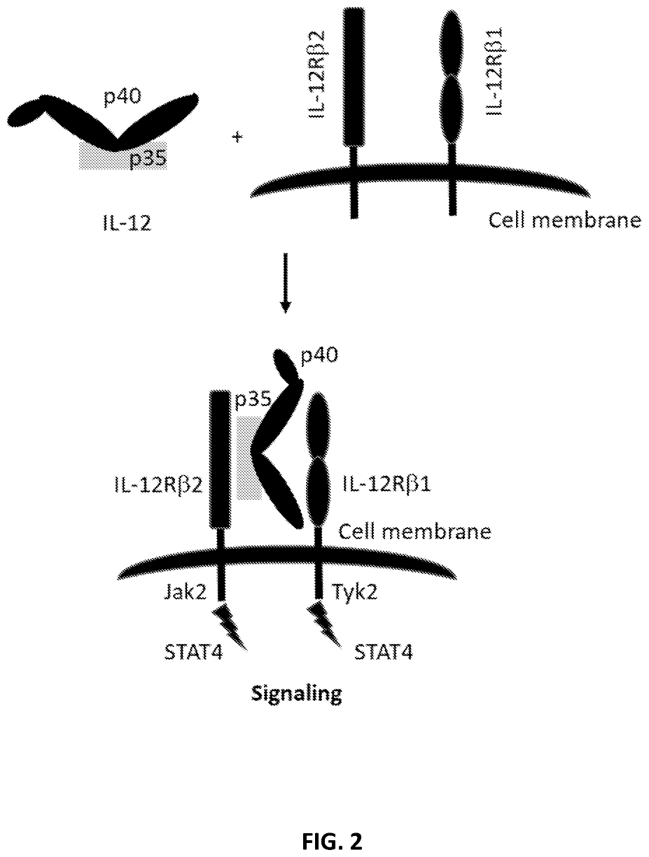

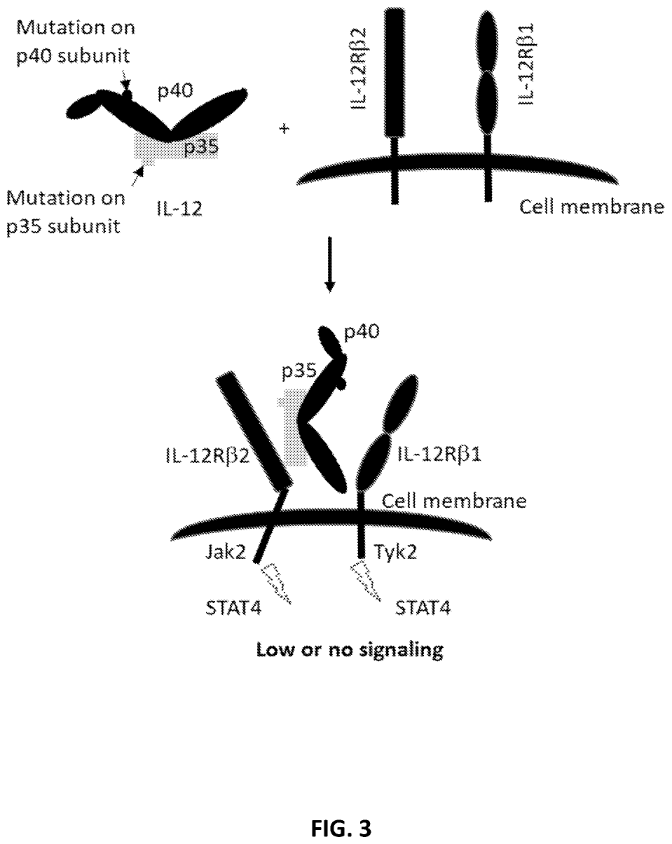

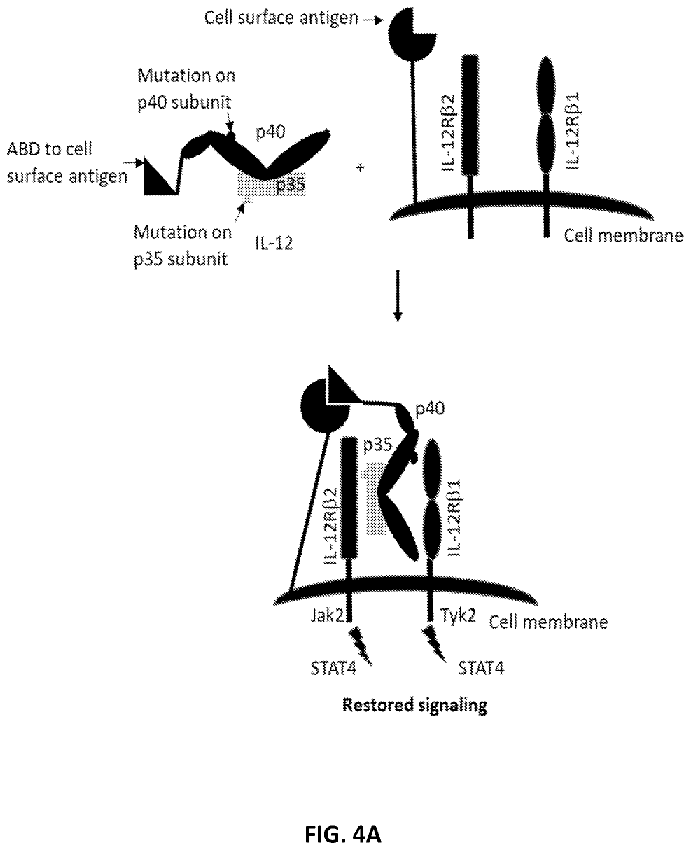

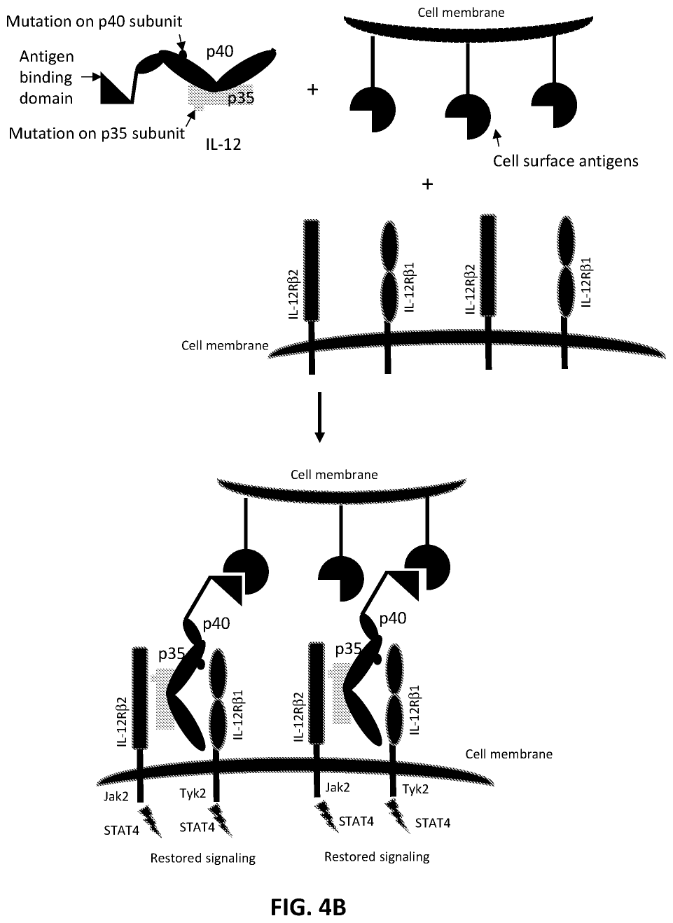

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “attenuated protein” refers to a protein or protein complex that comprises an active domain which has inherently reduced biological activity (for example, receptor binding affinity, signaling activity) relative to its corresponding wild-type protein or protein complex under comparable conditions.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological or chemical agent that interferes with or otherwise reduces the physiological action of another agent or molecule. In some instances, the antagonist specifically binds to the other agent or molecule. Included are full and partial antagonists.

An “agonist” refers to biological or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction.

Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (e.g., homodimer, heterodimer) as described herein at which it induces a response halfway between the baseline and maximum after some specified exposure time; the EC₅₀ of a graded dose response curve therefore represents the concentration of a compound at which 50% of its maximal effect is observed. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC₅₀ of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, efferocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7, 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

The term “isolated” polypeptide or protein (the terms “polypeptide” and “protein” are used interchangeably) referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys, and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Attenuated and Activatable Protein Homodimers and Heterodimers

Embodiments of the present disclosure include attenuated protein homodimers and heterodimers, comprising IL-12A and IL-12B proteins that remain relatively inactive in the attenuated protein form, and which can be “activated” upon contact with the appropriate environment.

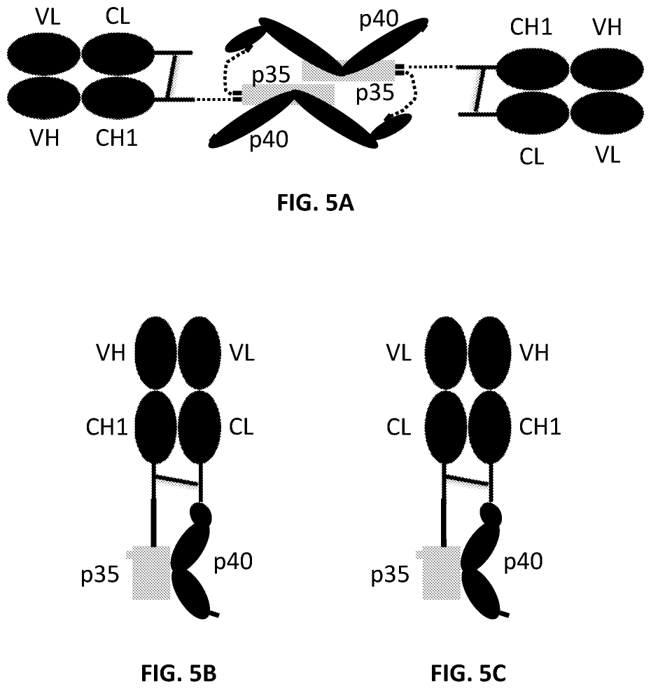

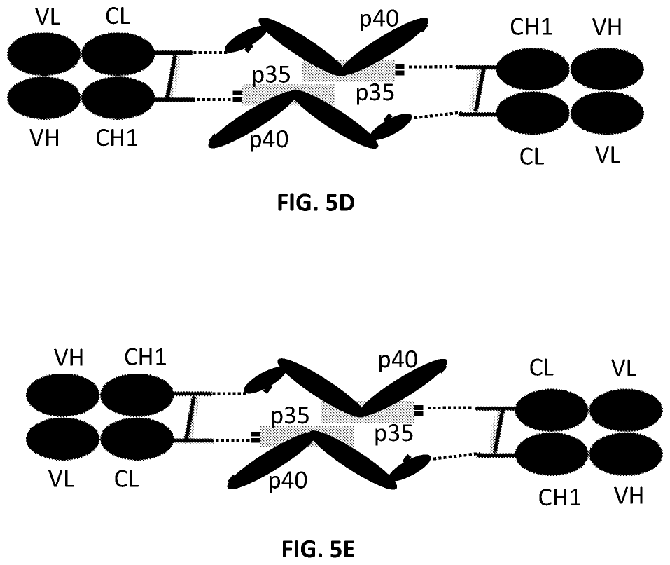

The attenuated protein homodimers described herein comprise at least two separate but otherwise identical polypeptide chains, which bind together via non-covalent interactions and/or certain covalent bonds, for example, disulfide bonds, but not via peptide or amide bonds. Generally, each polypeptide chain comprises a region of an immunoglobulin antigen binding domain (ABD) (for example, a light chain variable region, a heavy chain variable region), an IL-12A (p35) protein, an IL-12B (p40) protein, and at least one linker, for example, a flexible linker. The corresponding region(s) of the ABD are typically present as separate polypeptide chains, bound to the homodimer portion of the ABD via typical immunoglobulin interactions, including a disulfide bond (see, for example, FIG. 5A). The linker can be stable or degradable, for example, protease degradable. In some instances, the flexible linker is short enough to prevent or reduce intra-chain binding between the IL-12A and IL12B proteins, so that the IL-12A protein of the first polypeptide is bound to the IL-12B protein of the second polypeptide, and the IL-12A protein of the second polypeptide is bound to the IL-12B protein of the first polypeptide, to form a relatively stable homodimer in which these binding interactions sterically hinder or otherwise attenuate the IL-12 proteins in each chain from interacting with or binding to their cognate receptor(s) on an immune cell.

The attenuated protein heterodimers described herein comprise at least two separate and distinct or different polypeptide chains, which bind together via non-covalent interactions and/or certain covalent bonds, for example, disulfide bonds, but not via peptide or amide bonds (see FIGS. 5B-5C). One polypeptide chain comprises a first region of an immunoglobulin ABD (e.g., heavy chain variable region) and an IL-12A (p35) protein, and the other polypeptide chain comprises a second region of the ABD (e.g., light chain variable region) and an IL-12B (p40) protein, and optional linkers in between. The p35 and p40 proteins are bound together, and first and second regions of the ABD are bound together via a disulfide bond to form a functional ABD. At least one of the p35 protein and/or the P40 protein is a variant that has reduced binding affinity to the IL-12 co-receptors on the surface of an immune cell. Certain attenuated protein heterodimers comprise four polypeptide chains (two of the p35 polypeptide chains, and two of the p40 polypeptide chains), which bind together to form tetramers (see FIGS. 5D-5E). In certain of these and related embodiments, the linkers are relatively short, for example, about or less than about 1, 2, 3, 4, 5, 6, 7, 8, 9, or 10 amino acids in length.

As noted above, the homodimers and heterodimers described herein comprise at least one immunoglobulin ABD. Typically, the ABD has binding specificity (that is, it specifically binds) to a cell surface receptor on a cell, for example, cell surface receptor that is not an IL-12 receptor but is co-expressed on an immune cell with the IL-12 co-receptor complex. In some instances, the cell surface receptor is inducible or conditionally-expressed under appropriate conditions, for example, diseased conditions such as a tumor microenvironment. If an immune cell expresses only the IL-12 co-receptors (and not the targeted cell surface receptor), the attenuated protein homodimers and heterodimers at best bind weakly to the cell and initiate little to no signaling activity. However, if an immune cell expresses the IL-12 co-receptors along with the targeted cell surface receptor, then binding of the ABD to the cell surface receptor limits the diffusivity property of the attenuated protein homodimer or heterodimer, increases its binding (avidity) to the IL-12 co-receptors on the immune cell, and thereby increases at least one IL-12 signaling activity of the homodimer or heterodimer. In some instances, the ABD has binding specificity (that is, it specifically binds) to a plasma protein or an extracellular matrix (ECM) protein. Here, binding of the ABD to such target proteins likewise limits the diffusivity property of the homodimer or heterodimer, which increases the binding avidity of the IL-12 variant(s) to IL-12 co-receptors on a nearby immune cell, and thereby increases the IL-12 variant signaling activity of the homodimer or heterodimer. Thus, the attenuated protein homodimers and heterodimers described herein can be selectively activated against a sub-population of immune cells that co-express IL-12 co-receptors and the targeted cell surface protein, plasma protein, or ECM protein, for example, locally-activated immune cells in diseases or otherwise damaged tissues, including tumor tissues.

The attenuated protein homodimers and heterodimers described herein address many of the drawbacks of standard IL-12 therapies in the treatment of cancer, infectious diseases, and other diseases, including high initial serum Cₘₐₓ, which causes over-activation of the immune system, short PK because of the otherwise small molecular size of IL-12 and/or catabolism by the large number of immune cells that express IL-12 receptors, poor accumulation in the target tissues (e.g., cancers, tumors) because of the short PK and/or ineffective tumor targeting, and undesirable accumulation and immune activation in normal tissues.

Embodiments of the present disclosure thus include an attenuated protein homodimer, comprising a first polypeptide and a second polypeptide, wherein:
the first and the second polypeptide comprise, in an N- to C-terminal orientation, a region of an immunoglobulin antigen binding domain (ABD), an IL-12A (p35) protein, a linker, and an IL-12B (p40) protein (see, for example, FIG. 5A),
wherein the ABD specifically binds to a cell surface protein expressed on a cell, a plasma protein, or an extracellular matrix (ECM) protein, wherein the IL-12A protein of the first polypeptide is bound to the IL-12B protein of the second polypeptide, and wherein the IL-12B protein of the first polypeptide is bound to the IL-12A protein of the second polypeptide, wherein said binding partially masks a binding site of IL-12 protein(s) that otherwise binds to an IL-12Rβ1/IL-12Rβ2 receptor complex on the surface of an immune cell in vitro or in vivo and attenuates or reduces at least one IL-12 signaling activity of the homodimer relative to wild-type IL-12.

Also included is an attenuated protein heterodimer, comprising a first polypeptide and a second polypeptide, wherein:
(a) the first polypeptide comprises, in an N- to C-terminal orientation, a variable heavy chain (VH) region and a CH1 region, an optional linker, and an IL-12A (p35) protein, and the second polypeptide comprises, in an N- to C-terminal orientation, a variable light chain (VL) region and a CL region, an optional linker, and an IL-12B (p40) protein, wherein the p35 protein of the first polypeptide is bound to the p40 protein of the second polypeptide (see, for example, FIG. 5B); or
(b) the first polypeptide comprises, in an N- to C-terminal orientation, a variable heavy chain (VH) region and a CH1 region, an optional linker, and an IL-12B (p40) protein, and the second polypeptide comprises, in an N- to C-terminal orientation, a variable light chain (VL) region and a CL region, an optional linker, and an IL-12A (35) protein, wherein the p40 protein of the first polypeptide is bound to the p35 protein of the second polypeptide (see, for example, FIG. 5C),
wherein the CH1 region and the CL region of (a) or (b) are bound together via a disulfide bond to form an immunoglobulin antigen binding domain (ABD), wherein the ABD specifically binds to a cell surface protein expressed on a cell, a plasma protein, or an extracellular matrix (ECM) protein, and wherein at least one of the p35 and/or the p40 protein is a variant that has one or more amino acid alterations relative to a wild-type p35 or p40 sequence, and which has reduced binding affinity to a wild-type IL-12Rβ1/IL-12Rβ2 receptor complex on the surface of an immune cell in vitro or in vivo relative to that of the wild-type p35 and/or p40 sequence.

Certain of the attenuated protein heterodimers comprise four polypeptides selected from:
(i) two of the first and second polypeptides of (a); and
(ii) two of the first and second polypeptides of (b), wherein the four polypeptides are bound together to form an attenuated protein tetramer, that is, an oligomer composed of four monomers or subunits. In specific attenuated protein tetramers, the linkers are about or less than about 1, 2, 3, 4, 5, 6, 7, 8, 9, or 10 amino acids in length.

Exemplary IL-12A (p35) proteins, IL-12B (p40) proteins, ABDs to cell surface proteins, plasma proteins, and ECM proteins, and linkers are described elsewhere herein.

As noted above, the attenuated homodimer and heterodimer proteins have (inherently) reduced binding affinity to the wild-type IL-12Rβ1/IL-12Rβ2 receptor complex on the surface of an immune cell. In certain attenuated homodimer proteins, the homodimeric binding between the first and second polypeptides allosterically interferes with or inhibits the binding of the IL-12A and/or IL12B proteins to their target receptor, for example, cognate IL-12Rβ1/IL-12Rβ2 receptor complexes on the surface of an immune cell. In the attenuated heterodimer proteins, as noted above, at least one the IL-12A and/or IL12B protein is a low-affinity variant that has reduced binding affinity to the wild-type IL-12Rβ1/IL-12Rβ2 receptor complex on the surface of an immune cell. In these and related embodiments, and under conditions where the cell surface protein is not substantially co-expressed with the IL-12 co-receptors, the attenuated homodimer or heterodimer protein show no binding, or substantially no binding, or attenuated binding to its target (the IL-12Rβ1/IL-12Rβ2 receptor complex), or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding to its target, as compared to the binding of the wild-type IL-12 proteins alone, optionally for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater, optionally as measured in vivo or in a Target Displacement in vitro assay available in the art.

In some embodiments, as noted above, binding of the ABD to the cell surface protein, plasma protein, or ECM protein, increases binding (avidity) of the IL-12 protein(s) in the homodimer or heterodimer to the IL-12Rβ1/IL-12Rβ2 receptor complex on the surface of an immune cell in vitro or in vivo, and thereby increases at least one IL-12 signaling activity of the homodimer. In specific embodiments, binding of the ABD to the cell surface protein increases binding (avidity) of the IL-12 protein(s) in the homodimer or heterodimer to the IL-12Rβ1/IL-12Rβ2 receptor complex by about or at least about 10%, 20%, 30%, 40%, 50%, 60%, 70%, 80%, 90%, 100%, 200%, 300%, 400%, 500%, 1000%, 2000%, 3000%, 4000%, or 5000% or more. In particular embodiments, binding of the ABD to the cell surface protein increases at least one IL-12 signaling activity of the homodimer or heterodimer by about or at least about 10%, 20%, 30%, 40%, 50%, 60%, 70%, 80%, 90%, 100%, 200%, 300%, 400%, 500%, 1000%, 2000%, 3000%, 4000%, or 5000% or more. In some embodiments, the at least one IL-12 signaling activity is selected from one or more of stimulating growth and function of T cells, enhancing cytotoxic activity of NK cells and/or CD8+ T cells, stimulating production of interferon-γ (IFN-γ) and/or tumor necrosis factor-α (TNF-α), and inhibiting angiogenesis.

In some instances, the various components of each polypeptide chain can be fused in any orientation or combination. For example, in some attenuated protein homodimers, the ABD comprises a (i) variable heavy chain (VH) region and a CH1 region, and (ii) a variable light chain (VL) region and a CL region, including wherein the CH1 region and the CL region are bound together via a disulfide bond. In certain of these and related embodiments, the C-terminus of the CH1 region is fused to the N-terminus of the p35 protein, optionally via a linker, and the VL/CL region is a separate polypeptide chain that is bound to the VH/CH1 region via the disulfide bond. In some embodiments, the C-terminus of the CL region is fused to the N-terminus of the p35 protein, optionally via a linker, and the VH/CH1 region is a separate polypeptide chain that is bound to the VL/CL region via the disulfide bond.

Certain attenuated protein homodimers and heterodimers are composed only of two of the foregoing protein chains, that is, they are composed only of a first polypeptide and a second polypeptide, as described herein. In some instances, however, certain homodimers and heterodimers comprise multiple chains, for example, where the first and second polypeptide chains form a “core structure” upon which additional or higher-order structures can be built, the various core structures being optionally bound together via additional protein binding domains, for example, tumor-targeting domains, serum albumin, human serum albumin binding domains, cell-surface targeted binding domains, matrix protein binding domains, and others.

The individual components of the attenuated protein homodimers and heterodimers are described in greater detail herein.

Interleukin-12 (IL-12) Proteins. The attenuated protein homodimers and heterodimers described herein comprise at least one “IL-12 protein”. IL-12 is a cytokine produced by dendritic cells, macrophages, neutrophils, and human B-lymphoblastoid cells (NC-37) in response to antigenic stimulation. IL-12 binds to the IL-12 receptor, which is a heterodimeric receptor formed by IL-12Rβ1 and IL-12Rβ2.

IL-12 is a heterodimeric cytokine encoded by two separate genes, IL-12A (p35) and IL-12B (p40). The active heterodimer (referred to as “p70”) is formed following protein synthesis.

Exemplary IL-12A (p35) polypeptide sequences are provided in Table S1.

TABLE S1
Exemplary IL-12A (p35) Polypeptides

| | | SEQ ID |
|---|---|---|
| Name | Sequence | NO. |
| Human IL- | MCPARSLLLVATLVLLDHLSLARNLPVATPDPGMFPCLHHSQNLLRAVSN | 1 |
| 12A (P35) | MLQKARQTLEFYPCTSEEIDHEDITKDKTSTVEACLPLELTKNESCLNSR | |
| Full-length | ETSFITNGSCLASRKTSFMMALCLSSIYEDLKMYQVEFKTMNAKLLMDPK | |
| | RQIFLDQNMLAVIDELMQALNFNSETVPQKSSLEEPDFYKTKIKLCILLH | |
| | AFRIRAVTIDRVMSYLNAS | |
| Human IL- | RNLPVATPDPGMFPCLHHSQNLLRAVSNMLQKARQTLEFYPCTSEEIDHE | 2 |
| 12A (P35) | DITKDKTSTVEACLPLELTKNESCLNSRETSFITNGSCLASRKTSFMMAL | |
| Mature | CLSSIYEDLKMYQVEFKTMNAKLLMDPKRQIFLDQNMLAVIDELMQALNE | |
| | NSETVPQKSSLEEPDFYKTKIKLCILLHAFRIRAVTIDRVMSYLNAS | |
| Human IL- | RNLPVATPDPGMFPCLHHSQNLLRAVSNMLQKARQTLEFYPCTSEEIDHE | 3 |
| 12A (P35) | DITKDKTSTVEACLPLELTKNESSLNSRETSFITNGSCLASRKTSFMMAL | |
| Mature | CLSSIYEDLKMYQVEFKTMNAKLLMDPKRQIFLDQNMLAVIDELMQALNF | |
| variant | NSETVPQKSSLEEPDFYKTKIKLCILLHAFRIRAVTIDRVMSYLNAS | |
| Human IL- | RNLPVATPDPGMFPCLHHSQNLLRAVSNMLQKARQTLEFYPCTSEEIDHE | 4 |
| 12A (P35) | DITKDKTSTVEACLPLELTKNESALNSRETSFITNGSCLASRKTSFMMAL | |
| Mature | CLSSIYEDLKMYQVEFKTMNAKLLMDPKRQIFLDQNMLAVIDELMQALNF | |
| variant | NSETVPQKSSLEEPDFYKTKIKLCILLHAFRIRAVTIDRVMSYLNAS | |
| Human IL- | RNLPVATPDPGMFPCLHHSQNLLRAVSNMLqKARQTLEFYPCTSEEIDHE | 5 |
| 12A (P35) | DITKDKTSTVEACLPLELTKNESSLNSRETSFITNGSCLASRKTSFMMAL | |
| Mature | CLSSIYEDLKMYQVEFKTMNAKLLMDPKRQIFLDQNMLAVIDELMQALNF | |
| variant | NSETVPQKSSLEEPDFYKTKIKLCILLHAFRIRAVTIDRVMSYLNAA | |
| Human IL- | RNLPVATPDPGMFPCLHHSQNLLRAVSNMLQKARQTLEFYPCTSEEIDHE | 6 |
| 12A (P35) | DITKDKTSTVEACLPLELTKNESSLNSRETSFITNGSCLASRKTSFMMAL | |
| Mature | CLSSIYEDLKMYQVEFKTMNAKLLMDPKRQIFLDQNMLAVIDELMQALNF | |
| variant | NSETVPQKSSLEEPDFYKTKIKLCILLHAFRIRAVTIARVMSYLNAA | |
| Human IL- | RNLPVATPDPGMFPCLHHSQNLLRAVSNMLQKARQTLEFYPCTSEEIDHE | 7 |
| 12A (P35) | DITKDKTSTVEACLPLELTKNESSLNSRETSFITNGSCLASRKTSFMMAL | |
| Mature | CLSSIYEDLKMYQVEFKTMNAKLLMDPKRQIFLDQNMLAVIDELMQALNF | |
| variant | NSETVPQKSSLEEPDFAKTKIKLCILLHAFRIRAVTIDRVMSYLNAA | |
| Human IL- | RNLPVATPDPGMFPCLHHSQNLLRAVSNMLQKARQTLEFYPCTSEEIDHE | 8 |
| 12A (P35) | DITKDKTSTVEACLPLELTKNESSLNSRETSFITNGSCLASRKTSFMMAL | |
| Mature | CLSSIYEDLKMYQVEFKTMNAKLLMDPKRQIFLDQNMLAVIDELMQALNF | |
| variant | NSETVPQKSSLEEPDFDKTKIKLCILLHAFRIRAVTIDRVMSYLNAA | |

Thus, in certain embodiments, an IL-12A protein comprises, consists, or consists essentially of one or more amino acid sequences selected from Table S1, or an active variant or fragment thereof that is at least 80, 85, 90, 95, 98, or 100% identical to a sequence selected from Table S1. In particular embodiments, the IL-12A protein is a mature form of IL-12A, or an active variant or fragment thereof. In certain embodiments, an IL-12A protein is a variant that comprises or retains an amino acid substitution at C74, as defined by the mature IL-12A sequence, including C74A or C74S.

In certain embodiments, an IL-12A protein is a variant that comprises or retains an amino acid substitution at S197, as defined by the mature IL-12A sequence, including S197A. In some embodiments, an IL-12A protein comprises an additional alanine residue (A) at position 198. In certain embodiments, the IL-12A (p35) protein is a variant (e.g., a low binding-affinity variant) that has one or more amino acid alterations relative to a wild-type p35 sequence, and which has reduced binding affinity to a wild-type IL-12Rβ1/IL-12Rβ2 receptor complex on the surface of an immune cell in vitro or in vivo relative to that of the wild-type p35 sequence. In some embodiments, the p35 protein is a variant that has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p35 sequence. In some instances, the low binding-affinity variant has an amino acid substitution at any one or more of Y167, E38, F39, P41, K128, F166, and/or D188, as defined by the mature p35 sequence. In specific embodiments, the amino acid substitution is selected from any one or more of Y167A, Y167D, Y167E, Y167F, Y167G, Y167H, Y167I, Y167L, Y167N, Y167Q, Y167S, Y167T, and Y167V; E38A, E38D, E38F, E38G, E38H, E38I, E38K, E38L, E38M, E38N, E38P, E38Q, E38R, E38S, E38T, E38V, and E38W; F39A, F39D, F39E, F39G, F39H, F39I, F39K, F39L, F39M, F39N, F39P, F39Q, F39R, F39S, F39T, F39V, F39W, and F39Y; P41A, P41D, P41E, P41F, P41G, P41H, P41I, P41K, P41L, P41M, P41N, P41Q, P41R, P41S, P41T, P41V, P41W, and P41Y; K128A, K128D), K128E, K128F7, K128G, K128H, K128I, K128L, K128M, K128N, K128P, K128Q, K128R, K128S, K128T, K128V, K128W, and K128Y; F166A, F166D, F166E, F166G, F166H, F166I, F166K, F166L, F166M, F166N, F166P, F166Q, F166R, F166S, F166T, F166V, F166W, and F166Y; and D188A, including any combination of the foregoing. In some embodiments, the low-binding affinity p35 protein comprises, consists, or consists essentially of one or more amino acid sequences selected from Table S1, or an active variant or fragment thereof that is at least 80, 85, 90, 95, 98, or 100% identical to a sequence selected from Table 81, and which comprises or retains any one or more of the foregoing amino acid substitutions.

Exemplary IL-12B (p40) polypeptide sequences are provided in Table 82.

TABLE S2
Exemplary IL-12B Polypeptides

| | | SEQ ID |
|---|---|---|
| Name | Sequence | NO. |
| Human IL- | MCHQQLVISWFSLVFLASPLVAIWELKKDVYVVELDWYPDAPGEMVVLTC | 9 |
| 12B (P40) | DTPEEDGITWTLDQSSEVLGSGKTLTIQVKEFGDAGQYTCHKGGEVLSHS | |
| Full-length | LLLLHKKEDGIWSTDILKDQKEPKNKTFLRCEAKNYSGRFTCWWLTTIST | |
| | DLTFSVKSSRGSSDPQGVTCGAATLSAERVRGDNKEYEYSVECQEDSACP | |
| | AAEESLPIEVMVDAVHKLKYENYTSSFFIRDIIKPDPPKNLQLKPLKNSR | |
| | QVEVSWEYPDTWSTPHSYFSLTFCVQVQGKSKREKKDRVFTDKTSATVIC | |
| | RKNASISVRAQDRYYSSSWSEWASVPCS | |
| Human IL- | IWELKKDVYVVELDWYPDAPGEMVVLTCDTPEEDGITWTLDQSSEVLGSG | 10 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSACPAAEESLPIEVMVDAVHKLKYEN | |
| | YTSSFFIRDIIKPDPPKNLQLKPLKNSRQVEVSWEYPDTWSTPHSYESLT | |
| | FCVQVQGKSKREKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 11 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSACPAAEESLPIEVMVDAVHKLKYEN | |
| variant | YTSSFFIRDIIKPDPPKNLQLKPLKNSRQVEVSWEYPDTWSTPHSYFSLT | |
| | FCVQVQGQDQDEKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 12 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSAAPAAEESLPIEVMVDAVHKLKYEN | |
| variant | YTSSFFIRDIIKPDPPKNLQLKPLKNSRQVEVSWEYPDTWSTPHSYFSLT | |
| | FCVQVQGQDQDEKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 13 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSAAPAAEESLPIEVMVDAVHKLKYEN | |
| variant | YTSSFFIRDIIKPDPPKNLQLKPLKNSRQVEVSWEYPDTWSTPHSYESLT | |
| | FCVQVQGQDQDEKKDRVFTDKTSATVICRKNASISVRAQARYYSSSWSEW | |
| | ASVPCS | |
| Human IL- | IWELKKDVYVVELDWYPDAPGEMVVLTCDTPEEDGITWTLDQSSEVLGSG | 14 |
| 12B | KTLTIQVKEFGDAGQYTCHKGGEVLSHSLLLLHKKEDGIWSTDILKDQKE | |
| (P40) | PKNKTFLRCEAKNFSGRFTCWWLTTISTDLTFSVKSSRGSSDPQGVTCGA | |
| Mature | ATLSAERVRGDNKEYEYSVECQEDSAAPAAEESLPIEVMVDAVHKLKYEN | |
| variant | YTSSFFIRDIIKPDPPKNLQLKPLKNSRQVEVSWEYPDTWSTPHSYESLT | |
| | FCVQVQGQDQDEKKDRVFTDKTSATVICRKNASISVRAQDRYYSSSWSEW | |
| | ASVPC | |
| Human IL- | IWELKKDVYVVELDWYPDAPGEMVVLTCDTPEEDGITWTLDQSSEVLGSG | 15 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSAAPAAEESLPIEVMVDAVHKLKYEN | |
| variant | YTSSFFIRDIIKPDPPKNLQLKPLKNSRQVEVSWEYPDTWSTPHSYESLT | |
| | FSVQVQGQDQDEKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 16 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSAAPAAEESLPIEVMVDAVHKLKYEN | |
| variant | YTSSFFIRDIIKPDPPKNLQLKPLKNSRQVEVSWEYPDTWSTPHSYFSLT | |
| | FCVQVQGKSKREKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 17 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSAAPAAEESLPIEVMVDAVHKLKYEN | |
| variant | YTSSFFIRDIIKPDPPKNLQLKPLKNSRQVEVSWEYPDTWSTPHSYFSLT | |
| | FSVQVQGKSKREKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 18 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSACPAAEESLPIEVMVDAVHKLKYEN | |
| variant | YTSSFFIRDIIKPDPPKNLQLKPLKNSRQVEVSWEYPDTWSTPHSYFSLT | |
| | FSVQVQGQDQDEKKDRVFTDKTSATVICRKNASISVRAQDRYYSSSWSEW | |
| | ASVPCS | |
| Human IL- | IWELKKDVYVVELDWYPDAPGEMVVLTCDTPEEDGITWTLDQSSEVLGSG | 19 |
| 12B | KTLTIQVKEFGDAGQYTCHKGGEVLSHSLLLLHKKEDGIWSTDILKDQKE | |
| (P40) | PKNKTFLRCEAKNYSGRFTCWWLTTISTDLTFSVKSSRGSSDPQGVTCGA | |
| Mature | ATLSAERVRGDNKEYEYSVECQEDSACPAAEESLPIEVMVDAVHKLKYEN | |
| variant | YTSSFFIRDIIKPDPPKNLQLKPLKNSRQVEVSWEYPDTWSTPHSYFSLT | |
| | FSVQVQGKSKREKKDRVFTDKTSATVICRKNASISVRAQDRYYSSSWSEW | |
| | ASVPCS | |

Thus, in certain embodiments, an IL-12B protein comprises, consists, or consists essentially of one or more amino acid sequences selected from Table S2, or an active variant or fragment thereof that is at least 80, 85, 90, 95, 98, or 100% identical to a sequence selected from Table S2. In particular embodiments, the IL-12B protein is a mature form of IL-12B3, or an active variant or fragment thereof. In some embodiments, the IL-12B protein is a variant that comprises or retains amino acid substitutions at any one or more of Y144, C177, C252, K258, S259, K260, R261, and/or D290, including combinations thereof, as defined by the mature IL-12B sequence, including any one or more of Y114F, C177A, C252S, K258Q, S259D), K260Q, R261D), and/or D290A, including combinations thereof (e.g., QDQD at residues K258-R261).

In certain embodiments, the IL-12B (p40) protein is a variant (e.g., a low binding-affinity variant) that has one or more amino acid alterations relative to a wild-type p40 sequence, and which has reduced binding affinity to a wild-type IL-12Rβ1/IL-12Rβ2 receptor complex on the surface of an immune cell in vitro or in vivo relative to that of the wild-type p40 sequence. In some embodiments, the p40 protein is a variant that has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p40 sequence. In specific embodiments, the p40 protein has or retains an amino acid substitution at any one or more of D18, E59, K99, and/or K264, as defined by the mature p40 sequence

In some embodiments, an “active” or “activated” heterodimer, homodimer, IL-12A or IL-12B protein, or fragment or variant thereof, is characterized, for example, by its ability to bind to an IL-12RβI and/or IL-12Rβ2 receptor chain (e.g., IL-12Rβ1/IL-12Rβ2 complex) present on the surface of an immune cell in vitro or in vivo, and stimulate downstream IL-12 signaling activities. Certain exemplary IL-12 signaling activities include differentiation of naive T cells into Th1 cells, stimulating growth and function of T cells generally, activation of the cytotoxic activity of natural killer (NK) cells and cytotoxic T lymphocytes, and anti-angiogenic activities (i.e., reducing formation of new blood vessels), among other activities. Additional examples of IL-12 signaling activities include stimulating the production of interferon-gamma (IFN-γ) and tumor necrosis factor-alpha (TNF-α) from T cells and NK cells, and reducing IL-4 mediated suppression of IFN-γ.

In some instances, IL-12A and/or IL-12B binds to the IL-12Rβ2 subunit of IL-12 receptor, which is found on activated T cells and is stimulated by cytokines that promote Th1 cell development and inhibited by those that promote Th2 cell development. Upon binding, IL-12R-β2 becomes tyrosine phosphorylated and provides binding sites for kinases, Tyk2 and Jak2, which in turn activate transcription factor proteins such as STAT4. Thus, in some instances, an active or activated IL-12A and/or IL-12B polypeptide activates the JAK-STAT pathway. In some instances, an IL-12A and/or IL-12B polypeptide has an anti-cancer activity.

Any one or more of the foregoing IL-12 proteins can be combined with any of the other components described herein, for example, ABDs and linkers, and other optional protein domains, to generate one or more attenuated protein homodimers or heterodimers, or larger, multi-chain structures comprising the same.

Antigen Binding Domains (ABDs). The attenuated protein homodimers and heterodimers described herein comprise an immunoglobulin ABD, which specifically binds to a cell surface protein expressed on a cell (for example, an immune cell), a plasma protein, or an extracellular matrix (ECM) protein. In particular embodiments, the cell surface protein is inducible and co-expressed on an immune cell with the IL-12Rβ1/IL-12Rβ2 receptor complex. By “inducible” is meant that the cell surface protein is not substantially constitutively expressed on the immune cell, but is rather conditionally-expressed upon certain conditions, for example, activation of the immune cell, exposure of the immune cell to diseased tissue, including tumor tissues, among other conditions.

In certain embodiments, the cell surface protein is selected from Programmed cell death protein 1 (PD-1), Programmed death-ligand 1 (PD-L1), B7H3 (CD276), T cell immunoreceptor with Ig and ITIM domains (TIGIT), T-cell immunoglobulin and mucin-domain containing-3 (TIM-3), NKG2D, 4-1BB (CD137), CD3, CD4, CD8, CD25, and CD70. Exemplary antibodies against the foregoing cell surface proteins are known in the art.

PD-1 is a cell surface receptor that belongs to the immunoglobulin superfamily and is expressed on activated T cells, B cells, and myeloid. PD-1 interacts with two ligands, PD-L1 and PD-L2. Exemplary anti-PD-1 antibodies include nivolumab, pembrolizumab, PDR001, MK-3475, AMP-224, AMP-514, and pidilizumab, among others (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579). PD-L1 is expressed on macrophages, among other cells. Exemplary anti-PD-L1 antibodies include atezolizumab (MPDL3280A), avelumab (MSB0010718C), and durvalumab (MEDI4736), among others (see, e.g., U.S. Pat. Nos. 9,102,725; 9,393,301; 9,402,899; 9,439,962).

B7H3, or CD276, is an immune checkpoint molecule expressed on a variety of cells, including tumor cells. Exemplary anti-B7H3 antibodies include enoblituzumab (MGA271), omburtamab (8H9), MGC018, Orlotamab (MGD009), among others (see, for example, Yang et al., Int J Biol Sci. 16:1767-1773, 2020).

TIGIT is a co-inhibitory receptor that is found on the surface of a variety of lymphoid cells, and suppresses antitumor immunity, for example, via Tregs. Exemplary anti-TIGIT antibodies include tiragotumab and etigilimab, among others (see, e.g., Johnston et al., Cancer Cell. 26:923-37, 2014; and Solomon and Garrido-Laguna, Cancer Immunol Immunother. 67:1659-1667, 2018). TIM-3 is an immune checkpoint molecule expressed, for example, on the cell surface of IFNγ-producing or activated CD4+Th1 cells, CD8+Tc1 cells, monocytes, macrophages, and dendritic cells. Exemplary anti-TIM-3 antibodies include TSR-022, MBG453, and LY3321367, among others (see, for example, Acharya et al., Tim-3 finds its place in the cancer immunotherapy landscape, Journal for Immuno Therapy of Cancer. 2020; 8: e000911.doi:10.1136/jitc-2020-000911).

NKG2D is a transmembrane protein belonging to the NKG2 family of C-type lectin-like receptors, and is expressed one NK cells, γδ T cells, and CD8+αβ T cells, among others (see, for example, Bauer et al., Science. 285:727-9, 1999). Exemplary anti-NKG2D antibodies include NNC0142-0002, CX5, and B10G5, among others (see, for example, Allez et al., Gut. 66:1918-1925, 2017; Steigerwald et al., MAbs. 1:115-127, 2009; and Lu et al., Clin Cancer Res. 21:4819-30, 2015). 4-1BB, or CD137, is a member of the tumor necrosis factor (TNF) receptor family, and is expressed on activated T cells of both the CD4+ and CD8+ lineages. Exemplary anti-4-1BB antibodies include utomilumab (PF-05082566) and urelumab (663513), among others (see, for example, Qiao et al., Int J Biol Sci. 3:455-462, 2007; and Girard and Watts, Immunity. 49:791-3, 2018).

CD3 is a protein complex and T cell co-receptor that is involved in activating cytotoxic T cells (CD8+ naive T cells) and T helper cells (CD4+ naive T cells). It is expressed on the surface of mature T cells. Exemplary anti-CD3 antibodies include muromonab-CD3, otelixizumab, teplizumab, foralumab, and visilizumab, among others. CD4 is a glycoprotein expressed on the surface of immune cells such as T helper cells, monocytes, macrophages, and dendritic cells. It is a co-receptor of the T cell receptor (TCR) and assists the latter in communicating with antigen-presenting cells. Exemplary anti-CD4 antibodies include cedelizumab, clenoliximab, ibalizumab, keliximab, priliximab, tregalizumab, and zanolimumab, among others.

CD8 is a transmembrane glycoprotein that serves as a co-receptor for the T-cell receptor (TCR). Along with the TCR, the CD8 co-receptor plays a role in T cell signaling and aiding with cytotoxic T cell antigen interactions. CD8 is mainly expressed on the surface of cytotoxic T cells, but can also be found on natural killer cells, cortical thymocytes, and dendritic cells. Exemplary anti-CD8 antibodies are widely commercially available. CD25 (or interleukin-2 receptor alpha chain, IL-2Rα) is a type I transmembrane protein expressed on activated T cells, activated B cells, some thymocytes, myeloid precursors, and oligodendrocytes. Exemplary anti-CD25 antibodies include basiliximab, daclizumab, and inolimomab, among others. CD70 is a protein that is expressed on highly activated lymphocytes. Exemplary anti-CD70 antibodies include ARGX-110, cusatuzumab, and vorsetuzumab, among others (see, for example, Jacobs et al., Pharmacology & Therapeutics. 155:1-10, 2015; and Israel et al., Mol Cancer Ther. 4:2037-2044, 2005).

In certain embodiments, the plasma protein is selected from albumins, globulins, fibrinogens, and clotting factors. In specific embodiments, the albumin is human serum albumin. In some embodiments, the globulin is an alpha 1-globulin (for example, alpha 1-antitrypsin, alpha 1-antichymotrypsin, orosomucoid (acid glycoprotein), serum amyloid A, alpha 1-lipoprotein), an alpha-2 globulin (for example, haptoglobin, alpha-2α-globulin, α2-macroglobulin, ceruloplasmin, thyroxine-binding globulin, alpha 2-antiplasmin, protein C, alpha 2-lipoprotein, angiotensinogen, cortisol binding globulin, or vitamin D-binding protein), a beta-globulin (for example, beta-2 microglobulin, plasminogen, angiostatins, properdin, sex hormone-binding globulin, and transferrin), or a gamma-globulin (in some instances, excluding immunoglobulins).

In some embodiments, the ECM protein is selected from collagens, elastin proteins, fibronectin, and laminins. In some embodiments, the collagen is fibrillar collagen, for example, Type I, Type II, Type III, Type V, or Type XI fibrillar collagen, or non-fibrillar collagen, for example, Fibril Associated Collagens with Interrupted Triple Helices (FACIT) Type IX, Type XII, Type XIV, Type XIX, or Type XXI collagen, short chain Type VIII or Type X collagen, basement membrane Type IV collagen, multiplexin Type XV or Type XVIII collagen, Membrane Associated Collagens with Interrupted Triple Helices (MACIT) Type XIII or Type XVII collagen, microfibril forming Type VI collage, or anchoring fibril Type VII collagen. In some embodiments, the elastin protein is elastin (UniProt: P15502), fibrillin 1 (UniProt: P35555), fibrillin 2 (UniProt: P35556), or fibrillin 3 (UniProt: Q75N90). In some embodiments, the fibronectin (UniProt: P02751) is soluble plasma fibronectin, or insoluble cellular fibronectin. In some embodiments, the laminin is a lamin trimer selected from LAMA1, LAMA2, LAMA3, LAMA4, LAMA5, LAMB1, LAMB2, LAMB3, LAMB4, LAMC1, LAMC2, and LAMC3, and/or a laminin selected from laminin-111, laminin-211, laminin-121, laminin-221, laminin-332, laminin-3B32, laminin-311, laminin-321, laminin-411, laminin-421, laminin-511, laminin-521, laminin-213, laminin-423, laminin-522, and laminin-523.

Typically, an ABD comprises a (i) variable heavy chain (VH) region and a CH1 region, and (ii) a variable light chain (VL) region and a CL region, including wherein the CH1 region and the CL region are bound together via a disulfide bond. In some embodiments, an ABD is a fragment antigen-binding domain (Fab), a F(ab′)2 domain, or a whole antibody. Thus, in some embodiments, an ABD comprises an Fc region.

The ABD can be derived from or composed of any immunoglobulin molecule, and can bind to essentially any cell surface protein that is co-expressed on an immune cell with the IL-12 co-receptors, or any one or more of the plasma proteins or ECM proteins described herein. In some instances, its primary function is to bind to the cell surface protein, plasma protein, or ECM protein, reduce the diffusivity of the attenuated protein homodimer or heterodimer at or near the cell surface of an IL-12 co-receptor expressing cell, and thereby conditionally increase its binding (avidity) to the IL-12 co-receptors on the cell. Thus, the ABD specifically binds to the targeted cell surface receptor, plasma protein, or ECM protein. The ABD or antibody can be of essentially any type. As is well known in the art, an antibody is an immunoglobulin molecule capable of specific binding to a target, through at least one epitope recognition site, located in the variable region of the immunoglobulin molecule.

The term “antigen-binding fragment” as used herein refers to a polypeptide fragment that contains at least one CDR of an immunoglobulin heavy and/or light chain that binds to the target antigen of interest, that is, the cell surface protein. In this regard, an antigen-binding fragment of the herein described antibodies may comprise 1, 2, 3, 4, 5, or all 6 CDRs of a V_{H} and V_{L} sequence from antibodies that bind to a target antigen.

The binding properties of ABDs, antibodies, and antigen-binding fragments thereof can be quantified using methods well known in the art (see Davies et al., Annual Rev. Biochem. 59:439-473, 1990). In some embodiments, an ABD or an antibody or antigen-binding fragment thereof specifically binds to an antigen domain or an epitope of a cell surface receptor with an equilibrium dissociation constant that is about or ranges from about ≤10⁻⁷ M to about 10⁻⁸ M. In some embodiments, the equilibrium dissociation constant is about or ranges from about ≤10⁻⁹ M to about ≤10⁻¹⁰ M. In certain illustrative embodiments, an ABD or antibody or antigen-binding fragment thereof has an affinity (Kd or EC₅₀) for a cell surface receptor (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

An ABD or antibody is said to exhibit “specific binding” or “preferential binding” if it reacts or associates more frequently, more rapidly, with greater duration and/or with greater affinity with a particular cell, substance, or particular epitope (i.e., antigen domain) than it does with alternative cells, substances, or epitopes. An ABD or antibody “specifically binds” or “preferentially binds” to an antigen domain or epitope of a cell surface protein if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an ABD or antibody that specifically or preferentially binds to a specific epitope is an ABD or antibody that binds that specific epitope with greater affinity, avidity, more readily, and/or with greater duration than it binds to other epitopes. It is also understood by reading this definition that, for example, an ABD or antibody (or moiety or epitope) that specifically or preferentially binds to a first target may or may not specifically or preferentially bind to a second target. The term is also applicable where, for example, an ABD or antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of an ABD or antibody for an antigen domain or epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the accumulated strength of multiple affinities of individual non-covalent binding interactions, such as between a protein receptor and its ligand, and is commonly referred to as “functional affinity”.

ABDs and other antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

In certain embodiments, ABDs and antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, and ABDs derived from the same, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as ABD, Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH:VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

Where bispecific antibodies are to be used, or ABDs derived from the same, these may be conventional bispecific antibodies, which can be manufactured in a variety of ways (Holliger and Winter, Current Opinion Biotechnol. 4:446-449, 1993), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

In certain embodiments, the ABDs or antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies, and ABDs derived therefrom, preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the ABDs or antibodies are “chimeric” antibodies. In this regard, a chimeric antibody is comprised of an antigen-binding fragment of an antibody operably linked or otherwise fused to a heterologous Fc portion of a different antibody. In certain embodiments, the Fc domain or heterologous Fc domain is of human origin. In certain embodiments, the Fc domain or heterologous Fc domain is of mouse origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

It will be appreciated that any one or more of the ABDs can be combined with any of the other P35 proteins, P40 proteins, and linkers described herein, to generate one or more attenuated protein homodimers or heterodimers, or larger, multi-chain structures comprising the same.

Linkers. As noted above, in certain embodiments, an attenuated protein homodimer or heterodimer comprises one or more linkers, or peptide linkers, including flexible linkers. In some embodiments, a linker is a non-cleavable linker, that is, a physiologically-stable linker, or a stable linker. In some embodiments, a linkers is a cleavable linker, for example, a cleavable linker that comprises a protease cleavage site.

In some embodiments, a linker is about 1-50 1-40, 1-30, 1-20, 1-10, 1-5, 1-4, 1-3 amino acids in length, or about 1, 2, 3,4, 5,6, 7, 8,9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

Exemplary stable linker sequences, including flexible linkers, are provided in Table L1.

TABLE L1
Exemplary stable linkers

| | | SEQ ID |
|---|---|---|
| Name | Sequence | NO: |
| | [G]ₓ | |
| | [S]ₓ | |
| | [N]ₓ | |
| | [GS]ₓ | |
| | [GGS]ₓ | |
| | [GSS]ₓ | |
| | [GSGS]ₓ | 20 |
| | [GGSG]ₓ | 21 |
| | [GGGS]ₓ | 22 |
| | [GSGGG]ₓ | 23 |
| | [GGGGS]ₓ | 24 |
| | [GN]ₓ | |
| | [GGN]ₓ | |
| | [GNN]ₓ | |
| | [GNGN]ₓ | 25 |
| | [GGNG]ₓ | 26 |
| | [GGGN]ₓ | 27 |
| | [GGGGN]ₓ | 28 |
| | DGGGS | 29 |
| | TGEKP | 30 |
| | GGRR | 31 |
| | EGKSSGSGSESKVD | 32 |
| | KESGSVSSEQLAQFRSLD | 33 |
| | GGRRGGGS | 34 |
| | LRQRDGERP | 35 |
| | LRQKDGGGSERP | 36 |
| | LRQKD(GGGS)₂ERP | 37 |
| | GGGGENLYFQGGGGS | 38 |
| | GGGGSENLYFQGGGSGGGGS | 39 |
| | GGSPLGLAGGGS | |
| | GGSGGPLGLAGSGRSDNRGGA | 41 |
| | GGGGSGGGGSENLYFQGGGSGGGGS | 42 |
| | GGGGSPLGLAGSGRSDNRGSGGGGS | 43 |

where ₓ is 1, 2, 3, 4, 5, 6, 7, 8, 9, 10, 11, 12, 13, 14, 15, 16, 17, 18, 19, or 20

Thus, in certain embodiment, a stable and/or flexible linker is selected from Table L1. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

In some embodiments, a cleavable linker comprises at least one protease cleavage site, or is a low pH-sensitive linker. Suitable protease cleavages sites and self-cleaving peptides are known to the skilled person (see, e.g., Ryan et al., J. Gener. Virol. 78:699-722, 1997; and Scymczak et al., Nature Biotech. 5:589-594, 2004). In some embodiments, the protease cleavage site is cleavable by a protease selected from one or more of a metalloprotease, a serine protease, a cysteine protease, and an aspartic acid protease. In particular embodiments, the protease cleavage site is cleavable by a protease selected from one or more of MMP1, MMP2, MMP3, MMP4, MMP5, MMP6, MMP7, MMP8, MMP9, MMP10, MMP11, MMP12, MMP13, MMP14, TEV protease, matriptase, uPA, FAP, Legumain, PSA, Kallikrein, Cathepsin A, and Cathepsin B.

Exemplary cleavable linker sequences are provided in Table L2. PGP-24 DNA

TABLE L2
Exemplary cleavable linkers

| | | SEQ ID |
|---|---|---|
| Name | Sequence | NO: |
| | PLGLA | 44 |
| | GPLGVR | 45 |
| | SGRSDNH | 46 |
| | SGRSDNQ | 47 |
| | SGRSDNR | 48 |
| | SGRSDNS | 49 |
| | SGRSDNT | 50 |

Thus, in certain embodiment, a cleavable linker is selected from Table L2. In particular embodiments, a cleavable linker has a half life at pH 7.4, 25° C., for example, at physiological pH, human body temperature (e.g., in vivo, in serum, in a given tissue), of about or less than about 30 minutes, 1 hour, 2 hours, 3 hours, 4 hours, 5 hours, 6 hours, 12 hours, 18 hours, 24 hours, 36 hours, 48 hours, 72 hours, or 96 hours, or any intervening half-life.

It will be appreciated that any one or more of the foregoing linkers can be combined with any of p35 proteins, p40 proteins, and/or ABDs described herein, to generate one or more attenuated protein homodimers or heterodimers, or larger, multi-chain structures comprising the same

Additional Domains. Certain attenuated protein homodimers or heterodimers comprise one or more additional domains, for example, binding domains. In some embodiments, each of polypeptides in an attenuated protein homodimer or heterodimer further comprise one or more protein domains at a free terminus.

In particular embodiments, the protein domains are selected from one or more of cell receptor targeting moieties optionally bi-specific targeting moieties, antigen binding domains optionally bi-specific antigen binding domains, cell membrane receptor extracellular domains (ECDs), Fc domains, human serum albumin (HSA), Fc binding domains, HSA binding domains, cytokines, chemokines, and soluble protein ligands.

In some embodiments, the one or more additional protein domains can be used to form complexes of two, three, four, five, or more attenuated protein homodimers or heterodimers, which are bound to together via the additional domain(s).

Exemplary attenuated protein homodimers are provided in Table S3.

TABLE S
Exemplary Attenuated Fusion Proteins

| | | |
|---|---|---|
| | | SEQ ID |
| Name | Sequence | NO: |

P3074

| | | |
|---|---|---|
| Chains 1 | RNLPVATPDPGMFPCLHHSQNLLRAVSNMLQKARQTLEFYPCTSEEIDHEDI | 51 |
| and 2 | TKDKTSTVEACLPLELTKNESCLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITWTLDQSSEVLG | |
| | SGKTLTIQVKEFGDAGQYTCHKGGEVLSHSLLLLHKKEDGIWSTDILKDQKE | |
| | PKNKTFLRCEAKNYSGRFTCWWLTTISTDLTFSVKSSRGSSDPQGVTCGAAT | |
| | LSAERVRGDNKEYEYSVECQEDSACPAAEESLPIEVMVDAVHKLKYENYTSS | |
| | FFIRDIIKPDPPKNLQLKPLKNSRQVEVSWEYPDTWSTPHSYFSLTFCVQVQ | |
| | GQDQDEKKDRVFTDKTSATVICRKNASISVRAQDRYYSSSWSEWASVPCS | |

P3075

| | | |
|---|---|---|
| Chains 1 | RNLPVATPDPGMFPCLHHSQNLLRAVSNMLQKARQTLEFYPCTSEEIDHEDI | 52 |
| and 2 | TKDKTSTVEACLPLELTKNESCLNSRETSFITNGSCLASRKTSEMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITWTLDQS | |
| | SEVLGSGKTLTIQVKEFGDAGQYTCHKGGEVLSHSLLLLHKKEDGIWSTDIL | |
| | KDQKEPKNKTFLRCEAKNYSGRFTCWWLTTISTDLTFSVKSSRGSSDPQGVT | |
| | CGAATLSAERVRGDNKEYEYSVECQEDSACPAAEESLPIEVMVDAVHKLKYE | |
| | NYTSSFFIRDIIKPDPPKNLQLKPLKNSRQVEVSWEYPDTWSTPHSYESLTF | |
| | CVQVQGQDQDEKKDRVFTDKTSATVICRKNASISVRAQDRYYSSSWSEWASV | |
| | PCS | |

P3076

| | | |
|---|---|---|
| Chains 1 | RNLPVATPDPGMFPCLHHSQNLLRAVSNMLQKARQTLEFYPCTSEEIDHEDI | 53 |
| and 2 | TKDKTSTVEACLPLELTKNESCLNSRETSFITNGSCLASRKTSEMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSACPAAEESLPIEVMVDAVH | |
| | KLKYENYTSSFFIRDIIKPDPPKNLQLKPLKNSRQVEVSWEYPDTWSTPHSY | |
| | FSLTFCVQVQGQDQDEKKDRVFTDKTSATVICRKNASISVRAQDRYYSSSWS | |
| | EWASVPCS | |

P3077

| | | |
|---|---|---|
| Chains 1 | RNLPVATPDPGMFPCLHHSQNLLRAVSNMLQKARQTLEFYPCTSEEIDHEDI | 54 |
| and 2 | TKDKTSTVEACLPLELTKNESSLNSRETSFITNGSCLASRKTSEMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITWTLDQSSEVLG | |
| | SGKTLTIQVKEFGDAGQYTCHKGGEVLSHSLLLLHKKEDGIWSTDILKDQKE | |
| | PKNKTFLRCEAKNYSGRFTCWWLTTISTDLTFSVKSSRGSSDPQGVTCGAAT | |
| | LSAERVRGDNKEYEYSVECQEDSAAPAAEESLPIEVMVDAVHKLKYENYTSS | |
| | FFIRDIIKPDPPKNLQLKPLKNSRQVEVSWEYPDTWSTPHSYFSLTFCVQVQ | |
| | GQDQDEKKDRVFTDKTSATVICRKNASISVRAQDRYYSSSWSEWASVPCS | |

P3078

| | | |
|---|---|---|
| Chains 1 | RNLPVATPDPGMFPCLHHSQNLLRAVSNMLQKARQTLEFYPCTSEEIDHEDI | 55 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITWTLDQS | |
| | SEVLGSGKTLTIQVKEFGDAGQYTCHKGGEVLSHSLLLLHKKEDGIWSTDIL | |
| | KDQKEPKNKTFLRCEAKNYSGRFTCWWLTTISTDLTFSVKSSRGSSDPQGVT | |
| | CGAATLSAERVRGDNKEYEYSVECQEDSAAPAAEESLPIEVMVDAVHKLKYE | |
| | NYTSSFFIRDIIKPDPPKNLQLKPLKNSRQVEVSWEYPDTWSTPHSYESLTF | |
| | CVQVQGQDQDEKKDRVFTDKTSATVICRKNASISVRAQDRYYSSSWSEWASV | |
| | PCS | |

P3079

| | | |
|---|---|---|
| Chains 1 | RNLPVATPDPGMFPCLHHSQNLLRAVSNMLQKARQTLEFYPCTSEEIDHEDI | 56 |
| and 2 | TKDKTSTVEACLPLELTKNESSLNSRETSFITNGSCLASRKTSEMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSAAPAAEESLPIEVMVDAVH | |
| | KLKYENYTSSFFIRDIIKPDPPKNLQLKPLKNSRQVEVSWEYPDTWSTPHSY | |
| | FSLTFCVQVQGQDQDEKKDRVFTDKTSATVICRKNASISVRAQDRYYSSSWS | |
| | EWASVPCS | |

P3080

| | | |
|---|---|---|
| Chains 1 | RNLPVATPDPGMFPCLHHSQNLLRAVSNMLQKARQTLEFYPCTSEEIDHEDI | 57 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITWTLDQSSEVLG | |
| | SGKTLTIQVKEFGDAGQYTCHKGGEVLSHSLLLLHKKEDGIWSTDILKDQKE | |
| | PKNKTFLRCEAKNFSGRFTCWWLTTISTDLTFSVKSSRGSSDPQGVTCGAAT | |
| | LSAERVRGDNKEYEYSVECQEDSAAPAAEESLPIEVMVDAVHKLKYENYTSS | |
| | FFIRDIIKPDPPKNLQLKPLKNSRQVEVSWEYPDTWSTPHSYFSLTFCVQVQ | |
| | GQDQDEKKDRVFTDKTSATVICRKNASISVRAQDRYYSSSWSEWASVPCS | |

P3081

| | | |
|---|---|---|
| Chains 1 | RNLPVATPDPGMFPCLHHSQNLLRAVSNMLQKARQTLEFYPCTSEEIDHEDI | 58 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITWTLDQS | |
| | SEVLGSGKTLTIQVKEFGDAGQYTCHKGGEVLSHSLLLLHKKEDGIWSTDIL | |
| | KDQKEPKNKTFLRCEAKNFSGRFTCWWLTTISTDLTFSVKSSRGSSDPQGVT | |
| | CGAATLSAERVRGDNKEYEYSVECQEDSAAPAAEESLPIEVMVDAVHKLKYE | |
| | NYTSSFFIRDIIKPDPPKNLQLKPLKNSRQVEVSWEYPDTWSTPHSYFSLTF | |
| | CVQVQGQDQDEKKDRVFTDKTSATVICRKNASISVRAQDRYYSSSWSEWASV | |
| | PCS | |

P3082

| | | |
|---|---|---|
| Chains 1 | RNLPVATPDPGMFPCLHHSQNLLRAVSNMLQKARQTLEFYPCTSEEIDHEDI | 59 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNAS [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNFSGRFTCWWLTTISTDLTESVKSSRGSSD | |
| | PQGVTCGAATLSAERVRGDNKEYEYSVECQEDSAAPAAEESLPIEVMVDAVH | |
| | KLKYENYTSSFFIRDIIKPDPPKNLQLKPLKNSRQVEVSWEYPDTWSTPHSY | |
| | FSLTFCVQVQGQDQDEKKDRVFTDKTSATVICRKNASISVRAQDRYYSSSWS | |
| | EWASVPCS | |

P3083

| | | |
|---|---|---|
| Chains 1 | EPKSSDKTHTCPPCPAPEAAGGPSVFLFPPKPKDTLMISRTPEVTCVVVDVS | 60 |
| and 2 | HEDPEVKFNWYVDGVEVHNAKTKPREEQYNSTYRVVSVLTVLHQDWLNGKEY | |
| | KCKVSNKALAAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVE | |
| | SCSVMHEALHNHYTQKSLSLSPG [symbol] RNLPVATPDPGMFPCLH | |
| | HSQNLLRAVSNMLQKARQTLEFYPCTSEEIDHEDITKDKTSTVEACLPLELT | |
| | KNESSLNSRETSFITNGSCLASRKTSFMMALCLSSIYEDLKMYQVEFKTMNA | |
| | KLLMDPKRQIFLDQNMLAVIDELMQALNFNSETVPQKSSLEEPDFYKTKIKL | |
| | CILLHAFRIRAVTIDRVMSYLNAS [symbol] IWELKKDVYVVEL | |
| | DWYPDAPGEMVVLTCDTPEEDGITWTLDQSSEVLGSGKTLTIQVKEFGDAGQ | |
| | YTCHKGGEVLSHSLLLLHKKEDGIWSTDILKDQKEPKNKTFLRCEAKNYSGR | |
| | FTCWWLTTISTDLTFSVKSSRGSSDPQGVTCGAATLSAERVRGDNKEYEYSV | |
| | ECQEDSAAPAAEESLPIEVMVDAVHKLKYENYTSSFFIRDIIKPDPPKNLQL | |
| | KPLKNSRQVEVSWEYPDTWSTPHSYFSLTFCVQVQGQDQDEKKDRVFTDKTS | |
| | ATVICRKNASISVRAQDRYYSSSWSEWASVPCS | |

P3084

| | | |
|---|---|---|
| Chains 1 | EPKSSDKTHTCPPCPAPEAAGGPSVFLFPPKPKDTLMISRTPEVTCVVVDVS | 61 |
| and 2 | HEDPEVKFNWYVDGVEVHNAKTKPREEQYNSTYRVVSVLTVLHQDWLNGKEY | |
| | KCKVSNKALAAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPG [symbol] RNLPVATPDPGMFPCLH | |
| | HSQNLLRAVSNMLQKARQTLEFYPCTSEEIDHEDITKDKTSTVEACLPLELT | |
| | KNESSLNSRETSFITNGSCLASRKTSFMMALCLSSIYEDLKMYQVEFKTMNA | |
| | KLLMDPKRQIFLDQNMLAVIDELMQALNFNSETVPQKSSLEEPDFYKTKIKL | |
| | CILLHAFRIRAVTIDRVMSYLNAS [symbol] IWELKKDV | |
| | YVVELDWYPDAPGEMVVLTCDTPEEDGITWTLDQSSEVLGSGKTLTIQVKEF | |
| | GDAGQYTCHKGGEVLSHSLLLLHKKEDGIWSTDILKDQKEPKNKTFLRCEAK | |
| | NYSGRFTCWWLTTISTDLTFSVKSSRGSSDPQGVTCGAATLSAERVRGDNKE | |
| | YEYSVECQEDSAAPAAEESLPIEVMVDAVHKLKYENYTSSFFIRDIIKPDPP | |
| | KNLQLKPLKNSRQVEVSWEYPDTWSTPHSYFSLTFCVQVQGQDQDEKKDRVE | |
| | TDKTSATVICRKNASISVRAQDRYYSSSWSEWASVPCS | |

P3085

| | | |
|---|---|---|
| Chains 1 | EPKSSDKTHTCPPCPAPEAAGGPSVFLFPPKPKDTLMISRTPEVTCVVVDVS | 62 |
| and 2 | HEDPEVKFNWYVDGVEVHNAKTKPREEQYNSTYRVVSVLTVLHQDWLNGKEY | |
| | KCKVSNKALAAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSP [symbol] RNLPVATPDPGMFPCLH | |
| | HSQNLLRAVSNMLQKARQTLEFYPCTSEEIDHEDITKDKTSTVEACLPLELT | |
| | KNESSLNSRETSFITNGSCLASRKTSFMMALCLSSIYEDLKMYQVEFKTMNA | |
| | KLLMDPKRQIFLDQNMLAVIDELMQALNFNSETVPQKSSLEEPDFYKTKIKL | |
| | CILLHAFRIRAVTIDRVMSYLNAS [symbol] IWE | |
| | LKKDVYVVELDWYPDAPGEMVVLTCDTPEEDGITWTLDQSSEVLGSGKTLTI | |
| | QVKEFGDAGQYTCHKGGEVLSHSLLLLHKKEDGIWSTDILKDQKEPKNKTFL | |
| | RCEAKNYSGRFTCWWLTTISTDLTFSVKSSRGSSDPQGVTCGAATLSAERVR | |
| | GDNKEYEYSVECQEDSAAPAAEESLPIEVMVDAVHKLKYENYTSSFFIRDII | |
| | KPDPPKNLQLKPLKNSRQVEVSWEYPDTWSTPHSYFSLTFCVQVQGQDQDEK | |
| | KDRVFTDKTSATVICRKNASISVRAQDRYYSSSWSEWASVPCS | |

P30952158

| | | |
|---|---|---|
| Chains 1 | QVQLVQSGAEVKKPGASVKVSCKASGYTFTMNIIHWVRQAPGQGLEWMGWIH | 63 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKTHTCPPCPAPEAAGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALAAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] RNLPVATPDPGMFP | |
| | CLHHSQNLLRAVSNMLQKARQTLEFYPCTSEEIDHEDITKDKTSTVEACLPL | |
| | ELTKNESSLNSRETSFITNGSCLASRKTSFMMALCLSSIYEDLKMYQVEFKT | |
| | MNAKLLMDPKRQIFLDQNMLAVIDELMQALNFNSETVPQKSSLEEPDFYKTK | |
| | IKLCILLHAFRIRAVTIDRVMSYLNAS [symbol] IWEL | |
| | KKDVYVVELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATLSAERVRG | |
| | DNKEYEYSVECQEDSAAPAAEESLPIEVMVDAVHKLKYENYTSSFFIRDIIK | |
| | PDPPKNLQLKPLKNSRQVEVSWEYPDTWSTPHSYFSLTFCVQVQGQDQDEKK | |
| | DRVFTDKTSATVICRKNASISVRAQDRYYSSSWSEWASVPCS | |
| Chains 3 | EIVLTQSPGTLSLSPGERATLSCRASQSVSTSAYSYMHWYQQKPGQAPRLLI | 64 |
| and 4 | YLASNLESGIPDRFSGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P30962158

| | | |
|---|---|---|
| Chains 1 | QVQLVQSGAEVKKPGASVKVSCKASGYTFTMNIIHWVRQAPGQGLEWMGWIH | 65 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDK [symbol] RNLPVATPDPGMFPCLHHSQNLLRAVSNML | |
| | QKARQTLEFYPCTSEEIDHEDITKDKTSTVEACLPLELTKNESSLNSRETSF | |
| | ITNGSCLASRKTSFMMALCLSSIYEDLKMYQVEFKTMNAKLLMDPKRQIFLD | |
| | QNMLAVIDELMQALNFNSETVPQKSSLEEPDFYKTKIKLCILLHAFRIRAVT | |
| | IDRVMSYLNAS [symbol] IWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFCVQVQGQDQDEKKDRVFTDKTSATVICRK | |
| | NASISVRAQDRYYSSSWSEWASVPCS | |
| Chains 3 | EIVLTQSPGTLSLSPGERATLSCRASQSVSTSAYSYMHWYQQKPGQAPRLLI | 66 |
| and 4 | YLASNLESGIPDRFSGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P30972158

| | | |
|---|---|---|
| Chains 1 | QVQLVQSGAEVKKPGASVKVSCKASGYTFTMNIIHWVRQAPGQGLEWMGWIH | 67 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDK [symbol] RNLPVATPDPGMFPCLHHSQNLLRA | |
| | VSNMLQKARQTLEFYPCTSEEIDHEDITKDKTSTVEACLPLELTKNESSLNS | |
| | RETSFITNGSCLASRKTSFMMALCLSSIYEDLKMYQVEFKTMNAKLLMDPKR | |
| | QIFLDQNMLAVIDELMQALNFNSETVPQKSSLEEPDFYKTKIKLCILLHAFR | |
| | IRAVTIDRVMSYLNAS [symbol] IWELKKDVYVVELDW | |
| | YPDAPGEMVVLTCDTPEEDGITWTLDQSSEVLGSGKTLTIQVKEFGDAGQYT | |
| | CHKGGEVLSHSLLLLHKKEDGIWSTDILKDQKEPKNKTFLRCEAKNYSGRFT | |
| | CWWLTTISTDLTFSVKSSRGSSDPQGVTCGAATLSAERVRGDNKEYEYSVEC | |
| | QEDSAAPAAEESLPIEVMVDAVHKLKYENYTSSFFIRDIIKPDPPKNLQLKP | |
| | LKNSRQVEVSWEYPDTWSTPHSYFSLTFCVQVQGQDQDEKKDRVFTDKTSAT | |
| | VICRKNASISVRAQDRYYSSSWSEWASVPCS | |
| Chains 3 | EIVLTQSPGTLSLSPGERATLSCRASQSVSTSAYSYMHWYQQKPGQAPRLLI | 68 |
| and 4 | YLASNLESGIPDRFSGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P30982158

| | | |
|---|---|---|
| Chains 1 | QVQLVQSGAEVKKPGASVKVSCKASGYTFTMNIIHWVRQAPGQGLEWMGWIH | 69 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDK [symbol] RNLPVATPDPGMFPCLHHSQ | |
| | NLLRAVSNMLQKARQTLEFYPCTSEEIDHEDITKDKTSTVEACLPLELTKNE | |
| | SSLNSRETSFITNGSCLASRKTSFMMALCLSSIYEDLKMYQVEFKTMNAKLL | |
| | MDPKRQIFLDQNMLAVIDELMQALNFNSETVPQKSSLEEPDFYKTKIKLCIL | |
| | LHAFRIRAVTIDRVMSYLNAS [symbol] IWELKKDVYV | |
| | VELDWYPDAPGEMVVLTCDTPEEDGITWTLDQSSEVLGSGKTLTIQVKEFGD | |
| | AGQYTCHKGGEVLSHSLLLLHKKEDGIWSTDILKDQKEPKNKTFLRCEAKNY | |
| | SGRFTCWWLTTISTDLTFSVKSSRGSSDPQGVTCGAATLSAERVRGDNKEYE | |
| | YSVECQEDSAAPAAEESLPIEVMVDAVHKLKYENYTSSFFIRDIIKPDPPKN | |
| | LQLKPLKNSRQVEVSWEYPDTWSTPHSYFSLTFCVQVQGQDQDEKKDRVFTD | |
| | KTSATVICRKNASISVRAQDRYYSSSWSEWASVPCS | |
| Chains 3 | EIVLTQSPGTLSLSPGERATLSCRASQSVSTSAYSYMHWYQQKPGQAPRLLI | 70 |
| and 4 | YLASNLESGIPDRFSGSGSGTDFTLTISRLEPEDFAVYYCQQSRELPYTEGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P30991942

| | | |
|---|---|---|
| Chains 1 | QVQLVQSGAEVKKPGSSVKVSCKTSGDTFSTYAISWVRQAPGQGLEWMGGII | 71 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRVESKYGPPCPPCPAPEFLGGPSVFLFPPKPKDTLMISRTP | |
| | EVTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSRLTVD | |
| | KSRWQEGNVFSCSVMHEALHNHYTQKSLSLSL [symbol] RNLPVATPDPGMFP | |
| | CLHHSQNLLRAVSNMLQKARQTLEFYPCTSEEIDHEDITKDKTSTVEACLPL | |
| | ELTKNESSLNSRETSFITNGSCLASRKTSFMMALCLSSIYEDLKMYQVEFKT | |
| | MNAKLLMDPKRQIFLDQNMLAVIDELMQALNFNSETVPQKSSLEEPDFYKTK | |
| | IKLCILLHAFRIRAVTIDRVMSYLNAS [symbol] IWEL | |
| | KKDVYVVELDWYPDAPGEMVVLTCDTPEEDGITWTLDQSSEVLGSGKTLTIQ | |
| | VKEFGDAGQYTCHKGGEVLSHSLLLLHKKEDGIWSTDILKDQKEPKNKTFLR | |
| | CEAKNYSGRFTCWWLTTISTDLTFSVKSSRGSSDPQGVTCGAATLSAERVRG | |
| | DNKEYEYSVECQEDSAAPAAEESLPIEVMVDAVHKLKYENYTSSFFIRDIIK | |
| | PDPPKNLQLKPLKNSRQVEVSWEYPDTWSTPHSYFSLTFCVQVQGQDQDEKK | |
| | DRVFTDKTSATVICRKNASISVRAQDRYYSSSWSEWASVPCS | |
| Chains 3 | EIVLTQSPATLSLSPGERATLSCRASQSVSSYLAWYQQKPGQAPRLLIYDAS | 72 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P31001942

| | | |
|---|---|---|
| Chains 1 | QVQLVQSGAEVKKPGSSVKVSCKTSGDTFSTYAISWVRQAPGQGLEWMGGII | 73 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRV [symbol] RNLPVATPDPGMFPCLHHSQNLLRAVSNMLQKAR | |
| | QTLEFYPCTSEEIDHEDITKDKTSTVEACLPLELTKNESSLNSRETSFITNG | |
| | SCLASRKTSFMMALCLSSIYEDLKMYQVEFKTMNAKLLMDPKRQIFLDQNML | |
| | AVIDELMQALNFNSETVPQKSSLEEPDFYKTKIKLCILLHAFRIRAVTIDRV | |
| | MSYLNAS [symbol] IWELKKDVYVVELDWYPDAPGEMV | |
| | VLTCDTPEEDGITWTLDQSSEVLGSGKTLTIQVKEFGDAGQYTCHKGGEVLS | |
| | HSLLLLHKKEDGIWSTDILKDQKEPKNKTFLRCEAKNYSGRFTCWWLTTIST | |
| | DLTFSVKSSRGSSDPQGVTCGAATLSAERVRGDNKEYEYSVECQEDSAAPAA | |
| | EESLPIEVMVDAVHKLKYENYTSSFFIRDIIKPDPPKNLQLKPLKNSRQVEV | |
| | SWEYPDTWSTPHSYFSLTFCVQVQGQDQDEKKDRVFTDKTSATVICRKNASI | |
| | SVRAQDRYYSSSWSEWASVPCS | |
| Chains 3 | EIVLTQSPATLSLSPGERATLSCRASQSVSSYLAWYQQKPGQAPRLLIYDAS | 74 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P31011563

| | | |
|---|---|---|
| Chains 1 | EVQLLESGGGLVQPGGSLRLSCAASGFTFSSYAMSWVRQAPGKGLEWVSAII | 75 |
| and 2 | GSGASTYYADSVKGRFTISRDNSKNTLYLQMNSLRAEDTAVYYCAKGWFGGF | |
| | NYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKTHTCPPCPAPEAAGGPSVELFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALAAPIEKTISKAKGQPREPQVYTLPPSRDELTKNQVS | |
| | LTCLVKGFYPSDIAVEWESNGQPENNYKTTPPVLDSDGSFFLYSKLTVDKSR | |
| | WQQGNVFSCSVMHEALHNHYTQKSLSLSPG [symbol] RNLPVATPDPGMFPCLH | |
| | HSQNLLRAVSNMLQKARQTLEFYPCTSEEIDHEDITKDKTSTVEACLPLELT | |
| | KNESSLNSRETSFITNGSCLASRKTSEMMALCLSSIYEDLKMYQVEFKTMNA | |
| | KLLMDPKRQIFLDQNMLAVIDELMQALNFNSETVPQKSSLEEPDFYKTKIKL | |
| | CILLHAFRIRAVTIDRVMSYLNAS [symbol] IWELKKD | |
| | VYVVELDWYPDAPGEMVVLTCDTPEEDGITWTLDQSSEVLGSGKTLTIQVKE | |
| | FGDAGQYTCHKGGEVLSHSLLLLHKKEDGIWSTDILKDQKEPKNKTFLRCEA | |
| | KNYSGRFTCWWLTTISTDLTFSVKSSRGSSDPQGVTCGAATLSAERVRGDNK | |
| | EYEYSVECQEDSAAPAAEESLPIEVMVDAVHKLKYENYTSSFFIRDIIKPDP | |
| | PKNLQLKPLKNSRQVEVSWEYPDTWSTPHSYFSLTFCVQVQGQDQDEKKDRV | |
| | FTDKTSATVICRKNASISVRAQDRYYSSSWSEWASVPCS | |
| Chains 3 | EIVLTQSPGTLSLSPGERATLSCRASQSVTSSYLAWYQQKPGQAPRLLINVG | 76 |
| and 4 | SRRATGIPDRFSGSGSGTDFTLTISRLEPEDFAVYYCQQGIMLPPTFGQGTK | |
| | VEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTK | |
| | SFNRGEC | |

P31021563

| | | |
|---|---|---|
| Chains 1 | EVQLLESGGGLVQPGGSLRLSCAASGFTFSSYAMSWVRQAPGKGLEWVSAII | 77 |
| and 2 | GSGASTYYADSVKGRFTISRDNSKNTLYLQMNSLRAEDTAVYYCAKGWFGGF | |
| | NYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDK [symbol] RNLPVATPDPGMFPCLHHSQNLLRAVSNMLQKA | |
| | RQTLEFYPCTSEEIDHEDITKDKTSTVEACLPLELTKNESSLNSRETSFITN | |
| | GSCLASRKTSFMMALCLSSIYEDLKMYQVEFKTMNAKLLMDPKRQIFLDQNM | |
| | LAVIDELMQALNFNSETVPQKSSLEEPDFYKTKIKLCILLHAFRIRAVTIDR | |
| | VMSYLNAS [symbol] IWELKKDVYVVELDWYPDAPGEM | |
| | VVLTCDTPEEDGITWTLDQSSEVLGSGKTLTIQVKEFGDAGQYTCHKGGEVL | |
| | SHSLLLLHKKEDGIWSTDILKDQKEPKNKTFLRCEAKNYSGRFTCWWLTTIS | |
| | TDLTFSVKSSRGSSDPQGVTCGAATLSAERVRGDNKEYEYSVECQEDSAAPA | |
| | AEESLPIEVMVDAVHKLKYENYTSSFFIRDIIKPDPPKNLQLKPLKNSRQVE | |
| | VSWEYPDTWSTPHSYFSLTFCVQVQGQDQDEKKDRVFTDKTSATVICRKNAS | |
| | ISVRAQDRYYSSSWSEWASVPCS | |
| Chains 3 | EIVLTQSPGTLSLSPGERATLSCRASQSVTSSYLAWYQQKPGQAPRLLINVG | 78 |
| and 4 | SRRATGIPDRFSGSGSGTDFTLTISRLEPEDFAVYYCQQGIMLPPTFGQGTK | |
| | VEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTK | |
| | SFNRGEC | |

P3189

| | | |
|---|---|---|
| Chains 1 | RNLPVATPDPGMFPCLHHSQNLLRAVSNMLQKARQTLEFYPCTSEEIDHEDI | 79 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSAAPAAEESLPIEVMVDAVH | |
| | KLKYENYTSSFFIRDIIKPDPPKNLQLKPLKNSRQVEVSWEYPDTWSTPHSY | |
| | FSLTFCVQVQGQDQDEKKDRVFTDKTSATVICRKNASISVRAQDRYYSSSWS | |
| | EWASVPCS | |

P3190

| | | |
|---|---|---|
| Chains 1 | RNLPVATPDPGMFPCLHHSQNLLRAVSNMLQKARQTLEFYPCTSEEIDHEDI | 80 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSAAPAAEESLPIEVMVDAVH | |
| | KLKYENYTSSFFIRDIIKPDPPKNLQLKPLKNSRQVEVSWEYPDTWSTPHSY | |
| | FSLTFSVQVQGQDQDEKKDRVFTDKTSATVICRKNASISVRAQDRYYSSSWS | |
| | EWASVPCS | |

P3191

| | | |
|---|---|---|
| Chains 1 | RNLPVATPDPGMFPCLHHSQNLLRAVSNMLQKARQTLEFYPCTSEEIDHEDI | 81 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSAAPAAEESLPIEVMVDAVH | |
| | KLKYENYTSSFFIRDIIKPDPPKNLQLKPLKNSRQVEVSWEYPDTWSTPHSY | |
| | FSLTFCVQVQGKSKREKKDRVFTDKTSATVICRKNASISVRAQDRYYSSSWS | |
| | EWASVPCS | |

P3192

| | | |
|---|---|---|
| Chains 1 | RNLPVATPDPGMFPCLHHSQNLLRAVSNMLQKARQTLEFYPCTSEEIDHEDI | 82 |
| and 2 | TKDKTSTVEACLPLELTKNESSLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSAAPAAEESLPIEVMVDAVH | |
| | KLKYENYTSSFFIRDIIKPDPPKNLQLKPLKNSRQVEVSWEYPDTWSTPHSY | |
| | FSLTFSVQVQGKSKREKKDRVFTDKTSATVICRKNASISVRAQDRYYSSSWS | |
| | EWASVPCS | |

P3193

| | | |
|---|---|---|
| Chains 1 | RNLPVATPDPGMFPCLHHSQNLLRAVSNMLQKARQTLEFYPCTSEEIDHEDI | 83 |
| and 2 | TKDKTSTVEACLPLELTKNESCLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSACPAAEESLPIEVMVDAVH | |
| | KLKYENYTSSFFIRDIIKPDPPKNLQLKPLKNSRQVEVSWEYPDTWSTPHSY | |
| | FSLTFCVQVQGQDQDEKKDRVFTDKTSATVICRKNASISVRAQDRYYSSSWS | |
| | EWASVPCS | |

P3194

| | | |
|---|---|---|
| Chains 1 | RNLPVATPDPGMFPCLHHSQNLLRAVSNMLQKARQTLEFYPCTSEEIDHEDI | 84 |
| and 2 | TKDKTSTVEACLPLELTKNESCLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSACPAAEESLPIEVMVDAVH | |
| | KLKYENYTSSFFIRDIIKPDPPKNLQLKPLKNSRQVEVSWEYPDTWSTPHSY | |
| | FSLTFSVQVQGQDQDEKKDRVFTDKTSATVICRKNASISVRAQDRYYSSSWS | |
| | EWASVPCS | |

P3195

| | | |
|---|---|---|
| Chains 1 | RNLPVATPDPGMFPCLHHSQNLLRAVSNMLQKARQTLEFYPCTSEEIDHEDI | 85 |
| and 2 | TKDKTSTVEACLPLELTKNESCLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSACPAAEESLPIEVMVDAVH | |
| | KLKYENYTSSFFIRDIIKPDPPKNLQLKPLKNSRQVEVSWEYPDTWSTPHSY | |
| | FSLTFCVQVQGKSKREKKDRVFTDKTSATVICRKNASISVRAQDRYYSSSWS | |
| | EWASVPCS | |

P3196

| | | |
|---|---|---|
| Chains 1 | RNLPVATPDPGMFPCLHHSQNLLRAVSNMLQKARQTLEFYPCTSEEIDHEDI | 86 |
| and 2 | TKDKTSTVEACLPLELTKNESCLNSRETSFITNGSCLASRKTSFMMALCLSS | |
| | IYEDLKMYQVEFKTMNAKLLMDPKRQIFLDQNMLAVIDELMQALNFNSETVP | |
| | QKSSLEEPDFYKTKIKLCILLHAFRIRAVTIDRVMSYLNA [symbol] | |
| | [symbol] IWELKKDVYVVELDWYPDAPGEMVVLTCDTPEEDGITW | |
| | TLDQSSEVLGSGKTLTIQVKEFGDAGQYTCHKGGEVLSHSLLLLHKKEDGIW | |
| | STDILKDQKEPKNKTFLRCEAKNYSGRFTCWWLTTISTDLTFSVKSSRGSSD | |
| | PQGVTCGAATLSAERVRGDNKEYEYSVECQEDSACPAAEESLPIEVMVDAVH | |
| | KLKYENYTSSFFIRDIIKPDPPKNLQLKPLKNSRQVEVSWEYPDTWSTPHSY | |
| | FSLTFSVQVQGKSKREKKDRVFTDKTSATVICRKNASISVRAQDRYYSSSWS | |
| | EWASVPCS | |

P32692158

| | | |
|---|---|---|
| Chains 1 | QVQLVQSGAEVKKPGASVKVSCKASGYTFTMNIIHWVRQAPGQGLEWMGWIH | 87 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKTHTCPPCPAPEAAGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALAAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPG [symbol] RNLPVATPDPGMFP | |
| | CLHHSQNLLRAVSNMLQKARQTLEFYPCTSEEIDHEDITKDKTSTVEACLPL | |
| | ELTKNESSLNSRETSFITNGSCLASRKTSFMMALCLSSIYEDLKMYQVEFKT | |
| | MNAKLLMDPKRQIFLDQNMLAVIDELMQALNFNSETVPQKSSLEEPDFYKTK | |
| | IKLCILLHAFRIRAVTIDRVMSYLNAS [symbol] | |
| | IWELKKDVYVVELDWYPDAPGEMVVLTCDTPEEDGITWTLDQSSEVLGSGKT | |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSAAPAAEESLPIEVMVDAVHKLKYENYTSSFFIR | |
| | DIIKPDPPKNLQLKPLKNSRQVEVSWEYPDTWSTPHSYFSLTFSVQVQGQDQ | |
| | DEKKDRVFTDKTSATVICRKNASISVRAQDRYYSSSWSEWASVPCS | |
| Chains 3 | EIVLTQSPGTLSLSPGERATLSCRASQSVSTSAYSYMHWYQQKPGQAPRLLI | 88 |
| and 4 | YLASNLESGIPDRFSGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P32702158

| | | |
|---|---|---|
| Chains 1 | QVQLVQSGAEVKKPGASVKVSCKASGYTFTMNIIHWVRQAPGQGLEWMGWIH | 89 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDK [symbol] RNLPVATPDPGMFPCLHHSQNLLRAVSNML | |
| | QKARQTLEFYPCTSEEIDHEDITKDKTSTVEACLPLELTKNESSLNSRETSF | |
| | ITNGSCLASRKTSFMMALCLSSIYEDLKMYQVEFKTMNAKLLMDPKRQIFLD | |
| | QNMLAVIDELMQALNFNSETVPQKSSLEEPDFYKTKIKLCILLHAFRIRAVT | |
| | IDRVMSYLNAA [symbol] IWELKKDVYVVELDWY | |
| | PDAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVECQ | |
| | EDSAAPAAEESLPIEVMVDAVHKLKYENYTSSFFIRDIIKPDPPKNLQLKPL | |
| | KNSRQVEVSWEYPDTWSTPHSYFSLTFSVQVQGQDQDEKKDRVFTDKTSATV | |
| | ICRKNASISVRAQDRYYSSSWSEWASVPCS | |
| Chains 3 | EIVLTQSPGTLSLSPGERATLSCRASQSVSTSAYSYMHWYQQKPGQAPRLLI | 90 |
| and 4 | YLASNLESGIPDRFSGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P32712158

| | | |
|---|---|---|
| Chains 1 | QVQLVQSGAEVKKPGASVKVSCKASGYTFTMNIIHWVRQAPGQGLEWMGWIH | 91 |
| and 2 | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDK [symbol] RNLPVATPDPGMFPCLHHSQNLLRAVSNML | |
| | QKARQTLEFYPCTSEEIDHEDITKDKTSTVEACLPLELTKNESSLNSRETSF | |
| | ITNGSCLASRKTSFMMALCLSSIYEDLKMYQVEFKTMNAKLLMDPKRQIFLD | |
| | QNMLAVIDELMQALNFNSETVPQKSSLEEPDFYKTKIKLCILLHAFRIRAVT | |
| | IDRVMSYLNAA [symbol] IWELKKDVYVVELDWY | |
| | PDAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVECQ | |
| | EDSAAPAAEESLPIEVMVDAVHKLKYENYTSSFFIRDIIKPDPPKNLQLKPL | |
| | KNSRQVEVSWEYPDTWSTPHSYFSLTFSVQVQGQDQDEKKDRVFTDKTSATV | |
| | ICRKNASISVRAQDRYYSSSWSEWASVPCS | |
| Chains 3 | EIVLTQSPGTLSLSPGERATLSCRASQSVSTSAYSYMHWYQQKPGQAPRLLI | 92 |
| and 4 | YLASNLESGIPDRFSGSGSGTDFTLTISRLEPEDFAVYYCQQSRELPYTEGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |

P32721942

| | | |
|---|---|---|
| Chains 1 | QVQLVQSGAEVKKPGSSVKVSCKTSGDTFSTYAISWVRQAPGQGLEWMGGII | 93 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRVESKYGPPCPPCPAPEFLGGPSVFLFPPKPKDTLMISRTP | |
| | EVTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSRLTVD | |
| | KSRWQEGNVFSCSVMHEALHNHYTQKSLSLSLG [symbol] RNLPVATPDPGMFP | |
| | CLHHSQNLLRAVSNMLQKARQTLEFYPCTSEEIDHEDITKDKTSTVEACLPL | |
| | ELTKNESSLNSRETSFITNGSCLASRKTSFMMALCLSSIYEDLKMYQVEFKT | |
| | MNAKLLMDPKRQIFLDQNMLAVIDELMQALNFNSETVPQKSSLEEPDFYKTK | |
| | IKLCILLHAFRIRAVTIDRVMSYLNAA [symbol] | |
| | IWELKKDVYVVELDWYPDAPGEMVVLTCDTPEEDGITWTLDQSSEVLGSGKT | |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSAAPAAEESLPIEVMVDAVHKLKYENYTSSFFIR | |
| | DIIKPDPPKNLQLKPLKNSRQVEVSWEYPDTWSTPHSYFSLTFSVQVQGQDQ | |
| | DEKKDRVFTDKTSATVICRKNASISVRAQDRYYSSSWSEWASVPCS | |
| Chains 3 | EIVLTQSPATLSLSPGERATLSCRASQSVSSYLAWYQQKPGQAPRLLIYDAS | 94 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P32731942

| | | |
|---|---|---|
| Chains 1 | QVQLVQSGAEVKKPGSSVKVSCKTSGDTFSTYAISWVRQAPGQGLEWMGGII | 95 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRV [symbol] RNLPVATPDPGMFPCLHHSQNLLRAVSNMLQKAR | |
| | QTLEFYPCTSEEIDHEDITKDKTSTVEACLPLELTKNESSLNSRETSFITNG | |
| | SCLASRKTSFMMALCLSSIYEDLKMYQVEFKTMNAKLLMDPKRQIFLDQNML | |
| | AVIDELMQALNFNSETVPQKSSLEEPDFYKTKIKLCILLHAFRIRAVTIDRV | |
| | MSYLNAA [symbol] IWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chains 3 | EIVLTQSPATLSLSPGERATLSCRASQSVSSYLAWYQQKPGQAPRLLIYDAS | 96 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P32741942

| | | |
|---|---|---|
| Chains 1 | QVQLVQSGAEVKKPGSSVKVSCKTSGDTFSTYAISWVRQAPGQGLEWMGGII | 97 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRV [symbol] RNLPVATPDPGMFPCLHHSQNLLRAVSNMLQKAR | |
| | QTLEFYPCTSEEIDHEDITKDKTSTVEACLPLELTKNESSLNSRETSFITNG | |
| | SCLASRKTSFMMALCLSSIYEDLKMYQVEFKTMNAKLLMDPKRQIFLDQNML | |
| | AVIDELMQALNFNSETVPQKSSLEEPDFYKTKIKLCILLHAFRIRAVTIDRV | |
| | MSYLNAA [symbol] IWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chains 3 | EIVLTQSPATLSLSPGERATLSCRASQSVSSYLAWYQQKPGQAPRLLIYDAS | 98 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P32771942

| | | |
|---|---|---|
| Chains 1 | QVQLVQSGAEVKKPGSSVKVSCKTSGDTFSTYAISWVRQAPGQGLEWMGGII | 99 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRV [symbol] RNLPVATPDPGMFPCLHHSQNLLRAVSNMLQKAR | |
| | QTLEFYPCTSEEIDHEDITKDKTSTVEACLPLELTKNESSLNSRETSFITNG | |
| | SCLASRKTSFMMALCLSSIYEDLKMYQVEFKTMNAKLLMDPKRQIFLDQNML | |
| | AVIDELMQALNFNSETVPQKSSLEEPDFYKTKIKLCILLHAFRIRAVTIARV | |
| | MSYLNAA [symbol] IWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chains 3 | EIVLTQSPATLSLSPGERATLSCRASQSVSSYLAWYQQKPGQAPRLLIYDAS | 100 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P32781942

| | | |
|---|---|---|
| Chains 1 | QVQLVQSGAEVKKPGSSVKVSCKTSGDTFSTYAISWVRQAPGQGLEWMGGII | 101 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRV [symbol] RNLPVATPDPGMFPCLHHSQNLLRAVSNMLQKAR | |
| | QTLEFYPCTSEEIDHEDITKDKTSTVEACLPLELTKNESSLNSRETSFITNG | |
| | SCLASRKTSFMMALCLSSIYEDLKMYQVEFKTMNAKLLMDPKRQIFLDQNML | |
| | AVIDELMQALNFNSETVPQKSSLEEPDFAKTKIKLCILLHAFRIRAVTIDRV | |
| | MSYLNAA [symbol] IWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chains 3 | EIVLTQSPATLSLSPGERATLSCRASQSVSSYLAWYQQKPGQAPRLLIYDAS | 102 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

P32791942

| | | |
|---|---|---|
| Chains 1 | QVQLVQSGAEVKKPGSSVKVSCKTSGDTFSTYAISWVRQAPGQGLEWMGGII | 103 |
| and 2 | PIFGKAHYAQKFQGRVTITADESTSTAYMELSSLRSEDTAVYFCARKFHFVS | |
| | GSPFGMDVWGQGTTVTVSSASTKGPSVFPLAPCSRSTSESTAALGCLVKDYF | |
| | PEPVTVSWNSGALTSGVHTFPAVLQSSGLYSLSSVVTVPSSSLGTKTYTCNV | |
| | DHKPSNTKVDKRV [symbol] RNLPVATPDPGMFPCLHHSQNLLRAVSNMLQKAR | |
| | QTLEFYPCTSEEIDHEDITKDKTSTVEACLPLELTKNESSLNSRETSFITNG | |
| | SCLASRKTSFMMALCLSSIYEDLKMYQVEFKTMNAKLLMDPKRQIFLDQNML | |
| | AVIDELMQALNFNSETVPQKSSLEEPDFDKTKIKLCILLHAFRIRAVTIDRV | |
| | MSYLNAA [symbol] IWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chains 3 | EIVLTQSPATLSLSPGERATLSCRASQSVSSYLAWYQQKPGQAPRLLIYDAS | 104 |
| and 4 | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |

Thus, in certain embodiments, the first and second polypeptides (that is, chains 1 and 2) of an attenuated protein homodimer comprise, consist, or consist essentially of an amino acid sequence that is at least 80, 85, 90, 95, 98, or 100% identical to a sequence selected from Table S3. In certain embodiments, the attenuated proprotein homodimer comprises chains 3 and 4 selected from Table S3, for example, wherein the VL/CL region is a separate polypeptide chain that is bound to the VH/CH1 region via the disulfide bond, or wherein the VH/CH1 region is a separate polypeptide chain that is bound to the VL/CL region via the disulfide bond.

Exemplary attenuated protein heterodimers are provided in Table S4.

TABLE S4
Exemplary Attenuated Fusion Proteins

| | | |
|---|---|---|
| | | SEQ ID |
| Name | Sequence | NO: |

P32213218

| | | |
|---|---|---|
| Chain 1 | EVQLVESGGGLVQPGGSLRLSCAASGFTFSDSWIHWVRQAPGKGLEWVAWIS | 105 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] IWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSACPAAEESLPIEV | |
| | MVDAVHKLKYENYTSSFFIRDIIKPDPPKNLQLKPLKNSRQVEVSWEYPDTW | |
| | STPHSYFSLTFSVQVQGKSKREKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 106 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] RNLPVATPDPGMFPCLHHSQNLLRAVSNMLQKARQTLEFYP | |
| | CTSEEIDHEDITKDKTSTVEACLPLELTKNESCLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P3230313218

| | | |
|---|---|---|
| Chain 1 | IWELKKDVYVVELDWYPDAPGEMVVLTCDTPEEDGITWTLDQSSEVLGSGKT | 107 |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSACPAAEESLPIEVMVDAVHKLKYENYTSSFFIR | |
| | DIIKPDPPKNLQLKPLKNSRQVEVSWEYPDTWSTPHSYFSLTFCVQVQGKSK | |
| | REKKDRVFTDKTSATVICRKNASISVRAQDRYYSSSWSEWASVPCS | |
| Chain 2 | EVQLVESGGGLVQPGGSLRLSCAASGFTFSDSWIHWVRQAPGKGLEWVAWIS | 108 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK | |
| Chain 3 | DIQMTQSPSSLSASVGDRVTITCRASQDVSTAVAWYQQKPGKAPKLLIYSAS | 109 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] RNLPVATPDPGMFPCLHHSQNLLRAVSNMLQKARQTLEFYP | |
| | CTSEEIDHEDITKDKTSTVEACLPLELTKNESCLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P32223219

| | | |
|---|---|---|
| Chain 1 | EVQLVESGGGLVQPGGSLRLSCAASGFTFSDSWIHWVRQAPGKGLEWVAWIS | 110 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] IWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGKSKREKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 111 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] RNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P32233219

| | | |
|---|---|---|
| Chain 1 | EVQLVESGGGLVQPGGSLRLSCAASGFTFSDSWIHWVRQAPGKGLEWVAWIS | 112 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] IWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 113 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] RNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P32223220

| | | |
|---|---|---|
| Chain 1 | EVQLVESGGGLVQPGGSLRLSCAASGFTFSDSWIHWVRQAPGKGLEWVAWIS | 114 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] IWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 115 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] RNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |
| | A | |

P32233220

| | | |
|---|---|---|
| Chain 1 | EVQLVESGGGLVQPGGSLRLSCAASGFTFSDSWIHWVRQAPGKGLEWVAWIS | 116 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] IWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 117 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] RNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |
| | A | |

P32243227

| | | |
|---|---|---|
| Chain 1 | EVQLVESGGGLVQPGGSLRLSCAASGFTFSDSWIHWVRQAPGKGLEWVAWIS | 118 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] RNLPVATPDPGMFPCLHHSQNLLRAVSNMLQK | |
| | ARQTLEFYPCTSEEIDHEDITKDKTSTVEACLPLELTKNESCLNSRETSFIT | |
| | NGSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTID | |
| | RVMSYLNAS | |
| Chain 2 | DIQMTQSPSSLSASVGDRVTITCRASQDVSTAVAWYQQKPGKAPKLLIYSAS | 119 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] IWELKKDVYVVELDWYPDAPGEMVVLTCDTPEEDGITWTLD | |
| | QSSEVLGSGKTLTIQVKEFGDAGQYTCHKGGEVLSHSLLLLHKKEDGIWSTD | |
| | ILKDQKEPKNKTFLRCEAKNYSGRFTCWWLTTISTDLTFSVKSSRGSSDPQG | |
| | VTCGAATLSAERVRGDNKEYEYSVECQEDSACPAAEESLPIEVMVDAVHKLK | |
| | YENYTSSFFIRDIIKPDPPKNLQLKPLKNSRQVEVSWEYPDTWSTPHSYFSL | |
| | TFSVQVQGKSKREKKDRVFTDKTSATVICRKNASISVRAQDRYYSSSWSEWA | |
| | SVPCS | |

P3224302034

| | | |
|---|---|---|
| Chain 1 | EVQLVESGGGLVQPGGSLRLSCAASGFTFSDSWIHWVRQAPGKGLEWVAWIS | 120 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] RNLPVATPDPGMFPCLHHSQNLLRAVSNMLQK | |
| | ARQTLEFYPCTSEEIDHEDITKDKTSTVEACLPLELTKNESCLNSRETSFIT | |
| | NGSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTID | |
| | RVMSYLNAS | |
| Chain 2 | IWELKKDVYVVELDWYPDAPGEMVVLTCDTPEEDGITWTLDQSSEVLGSGKT | 121 |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSACPAAEESLPIEVMVDAVHKLKYENYTSSFFIR | |
| | DIIKPDPPKNLQLKPLKNSRQVEVSWEYPDTWSTPHSYFSLTFCVQVQGKSK | |
| | REKKDRVFTDKTSATVICRKNASISVRAQDRYYSSSWSEWASVPCS | |
| Chain 3 | DIQMTQSPSSLSASVGDRVTITCRASQDVSTAVAWYQQKPGKAPKLLIYSAS | 122 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC | |

P32253228

| | | |
|---|---|---|
| Chain 1 | EVQLVESGGGLVQPGGSLRLSCAASGFTFSDSWIHWVRQAPGKGLEWVAWIS | 123 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] RNLPVATPDPGMFPCLHHSQNLLRAVSNMLQK | |
| | ARQTLEFYPCTSEEIDHEDITKDKTSTVEACLPLELTKNESSLNSRETSFIT | |
| | NGSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTID | |
| | RVMSYLNAS | |
| Chain 2 | DIQMTQSPSSLSASVGDRVTITCRASQDVSTAVAWYQQKPGKAPKLLIYSAS | 124 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] IWELKKDVYVVELDWYPDAPGEMVVLTCDTPEEDGITWTLD | |
| | QSSEVLGSGKTLTIQVKEFGDAGQYTCHKGGEVLSHSLLLLHKKEDGIWSTD | |
| | ILKDQKEPKNKTFLRCEAKNYSGRFTCWWLTTISTDLTFSVKSSRGSSDPQG | |
| | VTCGAATLSAERVRGDNKEYEYSVECQEDSAAPAAEESLPIEVMVDAVHKLK | |
| | YENYTSSFFIRDIIKPDPPKNLQLKPLKNSRQVEVSWEYPDTWSTPHSYFSL | |
| | TFSVQVQGKSKREKKDRVFTDKTSATVICRKNASISVRAQDRYYSSSWSEWA | |
| | SVPCS | |

P32253229

| | | |
|---|---|---|
| Chain 1 | EVQLVESGGGLVQPGGSLRLSCAASGFTFSDSWIHWVRQAPGKGLEWVAWIS | 125 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] RNLPVATPDPGMFPCLHHSQNLLRAVSNMLQK | |
| | ARQTLEFYPCTSEEIDHEDITKDKTSTVEACLPLELTKNESSLNSRETSFIT | |
| | NGSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTID | |
| | RVMSYLNAS | |
| Chain 2 | DIQMTQSPSSLSASVGDRVTITCRASQDVSTAVAWYQQKPGKAPKLLIYSAS | 126 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] IWELKKDVYVVELDWYPDAPGEMVVLTCDTPEEDGITWTLD | |
| | QSSEVLGSGKTLTIQVKEFGDAGQYTCHKGGEVLSHSLLLLHKKEDGIWSTD | |
| | ILKDQKEPKNKTFLRCEAKNYSGRFTCWWLTTISTDLTFSVKSSRGSSDPQG | |
| | VTCGAATLSAERVRGDNKEYEYSVECQEDSAAPAAEESLPIEVMVDAVHKLK | |
| | YENYTSSFFIRDIIKPDPPKNLQLKPLKNSRQVEVSWEYPDTWSTPHSYFSL | |
| | TFSVQVQGQDQDEKKDRVFTDKTSATVICRKNASISVRAQDRYYSSSWSEWA | |
| | SVPCS | |

P32263228

| | | |
|---|---|---|
| Chain 1 | EVQLVESGGGLVQPGGSLRLSCAASGFTFSDSWIHWVRQAPGKGLEWVAWIS | 127 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] RNLPVATPDPGMFPCLHHSQNLLRAVSNMLQK | |
| | ARQTLEFYPCTSEEIDHEDITKDKTSTVEACLPLELTKNESSLNSRETSFIT | |
| | NGSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTID | |
| | RVMSYLNASA | |
| Chain 2 | DIQMTQSPSSLSASVGDRVTITCRASQDVSTAVAWYQQKPGKAPKLLIYSAS | 128 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] IWELKKDVYVVELDWYPDAPGEMVVLTCDTPEEDGITWTLD | |
| | QSSEVLGSGKTLTIQVKEFGDAGQYTCHKGGEVLSHSLLLLHKKEDGIWSTD | |
| | ILKDQKEPKNKTFLRCEAKNYSGRFTCWWLTTISTDLTFSVKSSRGSSDPQG | |
| | VTCGAATLSAERVRGDNKEYEYSVECQEDSAAPAAEESLPIEVMVDAVHKLK | |
| | YENYTSSFFIRDIIKPDPPKNLQLKPLKNSRQVEVSWEYPDTWSTPHSYFSL | |
| | TFSVQVQGKSKREKKDRVFTDKTSATVICRKNASISVRAQDRYYSSSWSEWA | |
| | SVPCS | |

P32263229

| | | |
|---|---|---|
| Chain 1 | EVQLVESGGGLVQPGGSLRLSCAASGFTFSDSWIHWVRQAPGKGLEWVAWIS | 129 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDK [symbol] RNLPVATPDPGMFPCLHHSQNLLRAVSNMLQK | |
| | ARQTLEFYPCTSEEIDHEDITKDKTSTVEACLPLELTKNESSLNSRETSFIT | |
| | NGSCLASRKTSFMMALCLSSIYEDLKMYQVEFKTMNAKLLMDPKRQIFLDQN | |
| | MLAVIDELMQALNFNSETVPQKSSLEEPDFYKTKIKLCILLHAFRIRAVTID | |
| | RVMSYLNASA | |
| Chain 2 | DIQMTQSPSSLSASVGDRVTITCRASQDVSTAVAWYQQKPGKAPKLLIYSAS | 130 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC [symbol] IWELKKDVYVVELDWYPDAPGEMVVLTCDTPEEDGITWTLD | |
| | QSSEVLGSGKTLTIQVKEFGDAGQYTCHKGGEVLSHSLLLLHKKEDGIWSTD | |
| | ILKDQKEPKNKTFLRCEAKNYSGRFTCWWLTTISTDLTFSVKSSRGSSDPQG | |
| | VTCGAATLSAERVRGDNKEYEYSVECQEDSAAPAAEESLPIEVMVDAVHKLK | |
| | YENYTSSFFIRDIIKPDPPKNLQLKPLKNSRQVEVSWEYPDTWSTPHSYFSL | |
| | TFSVQVQGQDQDEKKDRVFTDKTSATVICRKNASISVRAQDRYYSSSWSEWA | |
| | SVPCS | |

P35123513

| | | |
|---|---|---|
| Chain 1 | QVQLVESGGGVVQPGRSLRLDCKASGITFSNSGMHWVRQAPGKGLEWVAVIW | 131 |
| | YDGSKRYYADSVKGRFTISRDNSKNTLFLQMNSLRAEDTAVYYCATNDDYWG | |
| | QGTLVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVD | |
| | KKVEPKSCDKGGGGSIWELKKDVYVVELDWYPDAPGEMVVLTCDTPEEDGIT | |
| | WTLDQSSEVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSVKSSRGSS | |
| | DPQGVTCGAATLSAERVRGDNKEYEYSVECQEDSAAPAAEESLPIEVMVDAV | |
| | HKLKYENYTSSFFIRDIIKPDPPKNLQLKPLKNSRQVEVSWEYPDTWSTPHS | |
| | YFSLTFSVQVQGQDQDEKKDRVFTDKTSATVICRKNASISVRAQDRYYSSSW | |
| | SEWASVPCS | |
| Chain 2 | EIVLTQSPATLSLSPGERATLSCRASQSVSSYLAWYQQKPGQAPRLLIYDAS | 132 |
| | NRATGIPARFSGSGSGTDFTLTISSLEPEDFAVYYCQQSSNWPRTFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDEDKTKIKLCILLHAFRIRAVTIDRVMSYLNAS | |

P35123514

| | | |
|---|---|---|
| Chain 1 | QVQLVESGGGVVQPGRSLRLDCKASGITFSNSGMHWVRQAPGKGLEWVAVIW | 133 |
| | YDGSKRYYADSVKGRFTISRDNSKNTLFLQMNSLRAEDTAVYYCATNDDYWG | |
| | QGTLVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVD | |
| | KKVEPKSCDKGGGGSIWELKKDVYVVELDWYPDAPGEMVVLTCDTPEEDGIT | |
| | WTLDQSSEVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSVKSSRGSS | |
| | DPQGVTCGAATLSAERVRGDNKEYEYSVECQEDSAAPAAEESLPIEVMVDAV | |
| | HKLKYENYTSSFFIRDIIKPDPPKNLQLKPLKNSRQVEVSWEYPDTWSTPHS | |
| | YFSLTFSVQVQGQDQDEKKDRVFTDKTSATVICRKNASISVRAQDRYYSSSW | |
| | SEWASVPCS | |
| Chain 2 | EIVLTQSPATLSLSPGERATLSCRASQSVSSYLAWYQQKPGQAPRLLIYDAS | 134 |
| | NRATGIPARFSGSGSGTDFTLTISSLEPEDFAVYYCQQSSNWPRTFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFEKTKIKLCILLHAFRIRAVTIDRVMSYLNAS | |

P35123515

| | | |
|---|---|---|
| Chain 1 | QVQLVESGGGVVQPGRSLRLDCKASGITFSNSGMHWVRQAPGKGLEWVAVIW | 135 |
| | YDGSKRYYADSVKGRFTISRDNSKNTLFLQMNSLRAEDTAVYYCATNDDYWG | |
| | QGTLVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVD | |
| | KKVEPKSCDKGGGGSIWELKKDVYVVELDWYPDAPGEMVVLTCDTPEEDGIT | |
| | WTLDQSSEVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSVKSSRGSS | |
| | DPQGVTCGAATLSAERVRGDNKEYEYSVECQEDSAAPAAEESLPIEVMVDAV | |
| | HKLKYENYTSSFFIRDIIKPDPPKNLQLKPLKNSRQVEVSWEYPDTWSTPHS | |
| | YFSLTFSVQVQGQDQDEKKDRVFTDKTSATVICRKNASISVRAQDRYYSSSW | |
| | SEWASVPCS | |
| Chain 2 | EIVLTQSPATLSLSPGERATLSCRASQSVSSYLAWYQQKPGQAPRLLIYDAS | 136 |
| | NRATGIPARFSGSGSGTDFTLTISSLEPEDFAVYYCQQSSNWPRTFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFFKTKIKLCILLHAFRIRAVTIDRVMSYLNAS | |

P32233502

| | | |
|---|---|---|
| Chain 1 | EVQLVESGGGLVQPGGSLRLSCAASGFTESDSWIHWVRQAPGKGLEWVAWIS | 137 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSIWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 138 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFK | |
| | KTKIKLCILLHAFRIRAVTIDRVMSYLNAS | |

P32233503

| | | |
|---|---|---|
| Chain 1 | EVQLVESGGGLVQPGGSLRLSCAASGFTFSDSWIHWVRQAPGKGLEWVAWIS | 139 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSIWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 140 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDEMKTKIKLCILLHAFRIRAVTIDRVMSYLNAS | |

P32233504

| | | |
|---|---|---|
| Chain 1 | EVQLVESGGGLVQPGGSLRLSCAASGFTFSDSWIHWVRQAPGKGLEWVAWIS | 141 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSIWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 142 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFPKTKIKLCILLHAFRIRAVTIDRVMSYLNAS | |

P32233505

| | | |
|---|---|---|
| Chain 1 | EVQLVESGGGLVQPGGSLRLSCAASGFTFSDSWIHWVRQAPGKGLEWVAWIS | 143 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSIWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 144 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDERKTKIKLCILLHAFRIRAVTIDRVMSYLNAS | |

P32233506

| | | |
|---|---|---|
| Chain 1 | EVQLVESGGGLVQPGGSLRLSCAASGFTFSDSWIHWVRQAPGKGLEWVAWIS | 145 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSIWELKKDVYVVELDWYPDAPGEMVVLTCDTPE | |
| | EDGITWTLDQSSEVLGSGKTLTIQVKEFGDAGQYTCHKGGEVLSHSLLLLHK | |
| | KEDGIWSTDILKDQKEPKNKTFLRCEAKNYSGRFTCWWLTTISTDLTFSVKS | |
| | SRGSSDPQGVTCGAATLSAERVRGDNKEYEYSVECQEDSAAPAAEESLPIEV | |
| | MVDAVHKLKYENYTSSFFIRDIIKPDPPKNLQLKPLKNSRQVEVSWEYPDTW | |
| | STPHSYFSLTFSVQVQGQDQDEKKDRVFTDKTSATVICRKNASISVRAQDRY | |
| | YSSSWSEWASVPCS | |
| Chain 2 | DIQMTQSPSSLSASVGDRVTITCRASQDVSTAVAWYQQKPGKAPKLLIYSAS | 146 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFWKTKIKLCILLHAFRIRAVTIDRVMSYLNAS | |

P36673219

| | | |
|---|---|---|
| Chain 1 | EVQLVESGGGLVQPGGSLRLSCAASGFTFSDSWIHWVRQAPGKGLEWVAWIS | 147 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSSGRSDNRGGGGSIWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chain 2 | DIQMTQSPSSLSASVGDRVTITCRASQDVSTAVAWYQQKPGKAPKLLIYSAS | 148 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P36673668

| | | |
|---|---|---|
| Chain 1 | EVQLVESGGGLVQPGGSLRLSCAASGFTFSDSWIHWVRQAPGKGLEWVAWIS | 149 |
| | PYGGSTYYADSVKGRFTISADTSKNTAYLQMNSLRAEDTAVYYCARRHWPGG | |
| | FDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKGGGGSSGRSDNRGGGGSIWELKKDVYVVELDWYPDAP | |
| | GEMVVLTCDTPEEDGITWTLDQSSEVLGSGKTLTIQVKEFGDAGQYTCHKGG | |
| | EVLSHSLLLLHKKEDGIWSTDILKDQKEPKNKTFLRCEAKNYSGRFTCWWLT | |
| | TISTDLTFSVKSSRGSSDPQGVTCGAATLSAERVRGDNKEYEYSVECQEDSA | |
| | APAAEESLPIEVMVDAVHKLKYENYTSSFFIRDIIKPDPPKNLQLKPLKNSR | |
| | QVEVSWEYPDTWSTPHSYFSLTFSVQVQGQDQDEKKDRVFTDKTSATVICRK | |
| | NASISVRAQDRYYSSSWSEWASVPCS | |
| Chain 2 | DIQMTQSPSSLSASVGDRVTITCRASQDVSTAVAWYQQKPGKAPKLLIYSAS | 150 |
| | FLYSGVPSRFSGSGSGTDFTLTISSLQPEDFATYYCQQYLYHPATFGQGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSPLGLAGRNLPVATPDPGMFPCLHHSQNLLRAVSNMLQKARQ | |
| | TLEFYPCTSEEIDHEDITKDKTSTVEACLPLELTKNESSLNSRETSFITNGS | |
| | CLASRKTSFMMALCLSSIYEDLKMYQVEFKTMNAKLLMDPKRQIFLDQNMLA | |
| | VIDELMQALNFNSETVPQKSSLEEPDFYKTKIKLCILLHAFRIRAVTIDRVM | |
| | SYLNAS | |

P36693670

| | | |
|---|---|---|
| Chain 1 | QVQLVQSGAEVKKPGASVKVSCKASGYTFTMNIIHWVRQAPGQGLEWMGWIH | 151 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SAAPAAEESLPIEVMVDAVHKLKYENYTSSFFIRDIIKPDPPKNLQLKPLKN | |
| | SRQVEVSWEYPDTWSTPHSYFSLTFSVQVQGQDQDEKKDRVFTDKTSATVIC | |
| | RKNASISVRAQDRYYSSSWSEWASVPCS | |
| Chain 2 | EIVLTQSPGTLSLSPGERATLSCRASQSVSTSAYSYMHWYQQKPGQAPRLLI | 152 |
| | YLASNLESGIPDRESGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGECGGGGSRNLPVATPDPGMFPCLHHSQNLLRAVSNMLQKARQTL | |
| | EFYPCTSEEIDHEDITKDKTSTVEACLPLELTKNESSLNSRETSFITNGSCL | |
| | ASRKTSFMMALCLSSIYEDLKMYQVEFKTMNAKLLMDPKRQIFLDQNMLAVI | |
| | DELMQALNFNSETVPQKSSLEEPDFYKTKIKLCILLHAFRIRAVTIDRVMSY | |
| | LNAS | |

P36693671

| | | |
|---|---|---|
| Chain 1 | QVQLVQSGAEVKKPGASVKVSCKASGYTFTMNIIHWVRQAPGQGLEWMGWIH | 153 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SAAPAAEESLPIEVMVDAVHKLKYENYTSSFFIRDIIKPDPPKNLQLKPLKN | |
| | SRQVEVSWEYPDTWSTPHSYFSLTFSVQVQGQDQDEKKDRVFTDKTSATVIC | |
| | RKNASISVRAQDRYYSSSWSEWASVPCS | |
| Chain 2 | EIVLTQSPGTLSLSPGERATLSCRASQSVSTSAYSYMHWYQQKPGQAPRLLI | 154 |
| | YLASNLESGIPDRFSGSGSGTDFTLTISRLEPEDFAVYYCQQSRELPYTEGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGECGGGGSPLGLAGRNLPVATPDPGMFPCLHHSQNLLRAVSNMLQ | |
| | KARQTLEFYPCTSEEIDHEDITKDKTSTVEACLPLELTKNESSLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P37103712

| | | |
|---|---|---|
| Chain 1 | QVQLVQSGAEVKKPGSSVKVSCKTSGDTFSTYAISWVRQAPGQGLEWMGGII | 155 |
| | PIFGKAHYAQKFQGRVTITADESTSTAYMELSSLRSEDTAVYFCARKFHFVS | |
| | GSPFGMDVWGQGTTVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNV | |
| | NHKPSNTKVDKKVEPKSCDKGGGGSSGRSDNRGGGGSIWELKKDVYVVELDW | |
| | YPDAPGEMVVLTCDTPEEDGITWTLDQSSEVLGSGKTLTIQVKEFGDAGQYT | |
| | CHKGGEVLSHSLLLLHKKEDGIWSTDILKDQKEPKNKTFLRCEAKNYSGRFT | |
| | CWWLTTISTDLTFSVKSSRGSSDPQGVTCGAATLSAERVRGDNKEYEYSVEC | |
| | QEDSAAPAAEESLPIEVMVDAVHKLKYENYTSSFFIRDIIKPDPPKNLQLKP | |
| | LKNSRQVEVSWEYPDTWSTPHSYFSLTFSVQVQGQDQDEKKDRVFTDKTSAT | |
| | VICRKNASISVRAQDRYYSSSWSEWASVPCS | |
| Chain 2 | EIVLTQSPATLSLSPGERATLSCRASQSVSSYLAWYQQKPGQAPRLLIYDAS | 156 |
| | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGECGGGGSRNLPVATPDPGMFPCLHHSQNLLRAVSNMLQKARQTLEFYPC | |
| | TSEEIDHEDITKDKTSTVEACLPLELTKNESSLNSRETSFITNGSCLASRKT | |
| | SFMMALCLSSIYEDLKMYQVEFKTMNAKLLMDPKRQIFLDQNMLAVIDELMQ | |
| | ALNFNSETVPQKSSLEEPDFYKTKIKLCILLHAFRIRAVTIDRVMSYLNAS | |

P37113712

| | | |
|---|---|---|
| Chain 1 | QVQLVQSGAEVKKPGSSVKVSCKTSGDTFSTYAISWVRQAPGQGLEWMGGII | 157 |
| | PIFGKAHYAQKFQGRVTITADESTSTAYMELSSLRSEDTAVYFCARKFHFVS | |
| | GSPFGMDVWGQGTTVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNV | |
| | NHKPSNTKVDKKVEPKSCDKGGSPLGLAGLSGRSDNRGGGGSIWELKKDVYV | |
| | VELDWYPDAPGEMVVLTCDTPEEDGITWTLDQSSEVLGSGKTLTIQVKEFGD | |
| | AGQYTCHKGGEVLSHSLLLLHKKEDGIWSTDILKDQKEPKNKTFLRCEAKNY | |
| | SGRFTCWWLTTISTDLTFSVKSSRGSSDPQGVTCGAATLSAERVRGDNKEYE | |
| | YSVECQEDSAAPAAEESLPIEVMVDAVHKLKYENYTSSFFIRDIIKPDPPKN | |
| | LQLKPLKNSRQVEVSWEYPDTWSTPHSYFSLTFSVQVQGQDQDEKKDRVFTD | |
| | KTSATVICRKNASISVRAQDRYYSSSWSEWASVPCS | |
| Chain 2 | EIVLTQSPATLSLSPGERATLSCRASQSVSSYLAWYQQKPGQAPRLLIYDAS | 158 |
| | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGECGGGGSRNLPVATPDPGMFPCLHHSQNLLRAVSNMLQKARQTLEFYPC | |
| | TSEEIDHEDITKDKTSTVEACLPLELTKNESSLNSRETSFITNGSCLASRKT | |
| | SFMMALCLSSIYEDLKMYQVEFKTMNAKLLMDPKRQIFLDQNMLAVIDELMQ | |
| | ALNFNSETVPQKSSLEEPDFYKTKIKLCILLHAFRIRAVTIDRVMSYLNAS | |

P37133715

| | | |
|---|---|---|
| Chain 1 | QVQLVQSGSELKKPGASVKVSCKASGYTFTNYDINWVRQAPGQGLEWMGWIF | 159 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGGGSSGRSDNRGGGGSIWELKKDVYVVELDWYPDAPG | |
| | EMVVLTCDTPEEDGITWTLDQSSEVLGSGKTLTIQVKEFGDAGQYTCHKGGE | |
| | VLSHSLLLLHKKEDGIWSTDILKDQKEPKNKTFLRCEAKNYSGRFTCWWLTT | |
| | ISTDLTFSVKSSRGSSDPQGVTCGAATLSAERVRGDNKEYEYSVECQEDSAA | |
| | PAAEESLPIEVMVDAVHKLKYENYTSSFFIRDIIKPDPPKNLQLKPLKNSRQ | |
| | VEVSWEYPDTWSTPHSYFSLTFSVQVQGQDQDEKKDRVFTDKTSATVICRKN | |
| | ASISVRAQDRYYSSSWSEWASVPCS | |
| Chain 2 | DVVMTQSPAFLSVTPGEKVTITCRASQSISDYLYWYQQKPDQAPKLLIKYAS | 160 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P37143715

| | | |
|---|---|---|
| Chain 1 | QVQLVQSGSELKKPGASVKVSCKASGYTFTNYDINWVRQAPGQGLEWMGWIF | 161 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGSPLGLAGLSGRSDNRGGGGSIWELKKDVYVVELDWY | |
| | PDAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVECQ | |
| | EDSAAPAAEESLPIEVMVDAVHKLKYENYTSSFFIRDIIKPDPPKNLQLKPL | |
| | KNSRQVEVSWEYPDTWSTPHSYFSLTFSVQVQGQDQDEKKDRVFTDKTSATV | |
| | ICRKNASISVRAQDRYYSSSWSEWASVPCS | |
| Chain 2 | DVVMTQSPAFLSVTPGEKVTITCRASQSISDYLYWYQQKPDQAPKLLIKYAS | 162 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P37163717

| | | |
|---|---|---|
| Chain 1 | QVQLVQSGSELKKPGASVKVSCKASGYTFTNYDINWVRQAPGQGLEWMGWIF | 163 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKESKYGPPCPPCPAPEFLGGPSVFLFPPKPKDTLMISRT | |
| | PEVTCVVVDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPPCQEEMTK | |
| | NQVSLWCLVKGFYPSDIAVEWESNGQPENNYKTTPPVLDSDGSFFLYSRLTV | |
| | DKSRWQEGNVFSCSVMHEALHNHYTQKSLSLSLGGGGGSLSGRSDNRGGGGS | |
| | IWELKKDVYVVELDWYPDAPGEMVVLTCDTPEEDGITWTLDQSSEVLGSGKT | |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSAAPAAEESLPIEVMVDAVHKLKYENYTSSFFIR | |
| | DIIKPDPPKNLQLKPLKNSRQVEVSWEYPDTWSTPHSYFSLTFSVQVQGQDQ | |
| | DEKKDRVFTDKTSATVICRKNASISVRAQDRYYSSSWSEWASVPCS | |
| Chain 2 | DVVMTQSPAFLSVTPGEKVTITCRASQSISDYLYWYQQKPDQAPKLLIKYAS | 164 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECESKYGPPCPPCPAPEFLGGPSVFLFPPKPKDTLMISRTPEVTCVVV | |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVCTLPPSQEEMTKNQVSLSCA | |
| | VKGFYPSDIAVEWESNGQPENNYKTTPPVLDSDGSFFLVSRLTVDKSRWQEG | |
| | NVFSCSVMHEALHNHYTQKSLSLSLGGGGGSRNLPVATPDPGMFPCLHHSQN | |
| | LLRAVSNMLQKARQTLEFYPCTSEEIDHEDITKDKTSTVEACLPLELTKNES | |
| | SLNSRETSFITNGSCLASRKTSFMMALCLSSIYEDLKMYQVEFKTMNAKLLM | |
| | DPKRQIFLDQNMLAVIDELMQALNFNSETVPQKSSLEEPDFYKTKIKLCILL | |
| | HAFRIRAVTIDRVMSYLNAS | |

P37403715

| | | |
|---|---|---|
| Chain 1 | QVQLVQSGSELKKPGASVKVSCKASGYTFTNYDINWVRQAPGQGLEWMGWIF | 165 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGGGSSGRSDNRGGGGSIWELKKDVYVVELDWYPDAPG | |
| | EMVVLTCDTPEEDGITWTLDQSSEVLGSGKTLTIQVKEFGDAGQYTCHKGGE | |
| | VLSHSLLLLHKKEDGIWSTDILKDQKEPKNKTFLRCEAKNYSGRFTCWWLTT | |
| | ISTDLTFSVKSSRGSSDPQGVTCGAATLSAERVRGDNKEYEYSVECQEDSAA | |
| | PAAEESLPIEVMVDAVHKLKYENYTSSFFIRDIIKPDPPKNLQLKPLKNSRQ | |
| | VEVSWEYPDTWSTPHSYFSLTFSVQVQGKSKREKKDRVFTDKTSATVICRKN | |
| | ASISVRAQDRYYSSSWSEWASVPCS | |
| Chain 2 | DVVMTQSPAFLSVTPGEKVTITCRASQSISDYLYWYQQKPDQAPKLLIKYAS | 166 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P37413715

| | | |
|---|---|---|
| Chain 1 | QVQLVQSGSELKKPGASVKVSCKASGYTFTNYDINWVRQAPGQGLEWMGWIF | 167 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGSPLGLAGLSGRSDNRGGGGSIWELKKDVYVVELDWY | |
| | PDAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVECQ | |
| | EDSAAPAAEESLPIEVMVDAVHKLKYENYTSSFFIRDIIKPDPPKNLQLKPL | |
| | KNSRQVEVSWEYPDTWSTPHSYFSLTFSVQVQGKSKREKKDRVFTDKTSATV | |
| | ICRKNASISVRAQDRYYSSSWSEWASVPCS | |
| Chain 2 | DVVMTQSPAFLSVTPGEKVTITCRASQSISDYLYWYQQKPDQAPKLLIKYAS | 168 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

P37423717

| | | |
|---|---|---|
| Chain 1 | QVQLVQSGSELKKPGASVKVSCKASGYTFTNYDINWVRQAPGQGLEWMGWIF | 169 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKESKYGPPCPPCPAPEFLGGPSVFLFPPKPKDTLMISRT | |
| | PEVTCVVVDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPPCQEEMTK | |
| | NQVSLWCLVKGFYPSDIAVEWESNGQPENNYKTTPPVLDSDGSFFLYSRLTV | |
| | DKSRWQEGNVFSCSVMHEALHNHYTQKSLSLSLGGGGGSLSGRSDNRGGGGS | |
| | IWELKKDVYVVELDWYPDAPGEMVVLTCDTPEEDGITWTLDQSSEVLGSGKT | |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSAAPAAEESLPIEVMVDAVHKLKYENYTSSFFIR | |
| | DIIKPDPPKNLQLKPLKNSRQVEVSWEYPDTWSTPHSYFSLTFSVQVQGKSK | |
| | REKKDRVFTDKTSATVICRKNASISVRAQDRYYSSSWSEWASVPCS | |
| Chain 2 | DVVMTQSPAFLSVTPGEKVTITCRASQSISDYLYWYQQKPDQAPKLLIKYAS | 170 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECESKYGPPCPPCPAPEFLGGPSVFLFPPKPKDTLMISRTPEVTCVVV | |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVCTLPPSQEEMTKNQVSLSCA | |
| | VKGFYPSDIAVEWESNGQPENNYKTTPPVLDSDGSFFLVSRLTVDKSRWQEG | |
| | NVFSCSVMHEALHNHYTQKSLSLSLGGGGGSRNLPVATPDPGMFPCLHHSQN | |
| | LLRAVSNMLQKARQTLEFYPCTSEEIDHEDITKDKTSTVEACLPLELTKNES | |
| | SLNSRETSFITNGSCLASRKTSFMMALCLSSIYEDLKMYQVEFKTMNAKLLM | |
| | DPKRQIFLDQNMLAVIDELMQALNFNSETVPQKSSLEEPDFYKTKIKLCILL | |
| | HAFRIRAVTIDRVMSYLNAS | |

P37433717

| | | |
|---|---|---|
| Chain 1 | QVQLVQSGSELKKPGASVKVSCKASGYTFTNYDINWVRQAPGQGLEWMGWIF | 171 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKESKYGPPCPPCPAPEFLGGPSVFLFPPKPKDTLMISRT | |
| | PEVTCVVVDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPPCQEEMTK | |
| | NQVSLWCLVKGFYPSDIAVEWESNGQPENNYKTTPPVLDSDGSFFLYSRLTV | |
| | DKSRWQEGNVFSCSVMHEALHNHYTQKSLSLSLGGGSPLGLAGLSGRSDNRG | |
| | GGGSIWELKKDVYVVELDWYPDAPGEMVVLTCDTPEEDGITWTLDQSSEVLG | |
| | SGKTLTIQVKEFGDAGQYTCHKGGEVLSHSLLLLHKKEDGIWSTDILKDQKE | |
| | PKNKTFLRCEAKNYSGRFTCWWLTTISTDLTFSVKSSRGSSDPQGVTCGAAT | |
| | LSAERVRGDNKEYEYSVECQEDSAAPAAEESLPIEVMVDAVHKLKYENYTSS | |
| | FFIRDIIKPDPPKNLQLKPLKNSRQVEVSWEYPDTWSTPHSYFSLTFSVQVQ | |
| | GKSKREKKDRVFTDKTSATVICRKNASISVRAQDRYYSSSWSEWASVPCS | |
| Chain 2 | DVVMTQSPAFLSVTPGEKVTITCRASQSISDYLYWYQQKPDQAPKLLIKYAS | 172 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECESKYGPPCPPCPAPEFLGGPSVFLFPPKPKDTLMISRTPEVTCVVV | |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVCTLPPSQEEMTKNQVSLSCA | |
| | VKGFYPSDIAVEWESNGQPENNYKTTPPVLDSDGSFFLVSRLTVDKSRWQEG | |
| | NVFSCSVMHEALHNHYTQKSLSLSLGGGGGSRNLPVATPDPGMFPCLHHSQN | |
| | LLRAVSNMLQKARQTLEFYPCTSEEIDHEDITKDKTSTVEACLPLELTKNES | |
| | SLNSRETSFITNGSCLASRKTSFMMALCLSSIYEDLKMYQVEFKTMNAKLLM | |
| | DPKRQIFLDQNMLAVIDELMQALNFNSETVPQKSSLEEPDFYKTKIKLCILL | |
| | HAFRIRAVTIDRVMSYLNAS | |

P37633717

| | | |
|---|---|---|
| Chain 1 | QVQLVQSGSELKKPGASVKVSCKASGYTFTNYDINWVRQAPGQGLEWMGWIF | 173 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKESKYGPPCPPCPAPEFLGGPSVFLFPPKPKDTLMISRT | |
| | PEVTCVVVDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPPCQEEMTK | |
| | NQVSLWCLVKGFYPSDIAVEWESNGQPENNYKTTPPVLDSDGSFFLYSRLTV | |
| | DKSRWQEGNVFSCSVMHEALHNHYTQKSLSLSLGGGGGSIWELKKDVYVVEL | |
| | DWYPDAPGEMVVLTCDTPEEDGITWTLDQSSEVLGSGKTLTIQVKEFGDAGQ | |
| | YTCHKGGEVLSHSLLLLHKKEDGIWSTDILKDQKEPKNKTFLRCEAKNYSGR | |
| | FTCWWLTTISTDLTFSVKSSRGSSDPQGVTCGAATLSAERVRGDNKEYEYSV | |
| | ECQEDSAAPAAEESLPIEVMVDAVHKLKYENYTSSFFIRDIIKPDPPKNLQL | |
| | KPLKNSRQVEVSWEYPDTWSTPHSYFSLTFSVQVQGQDQDEKKDRVFTDKTS | |
| | ATVICRKNASISVRAQDRYYSSSWSEWASVPCS | |
| Chain 2 | DVVMTQSPAFLSVTPGEKVTITCRASQSISDYLYWYQQKPDQAPKLLIKYAS | 174 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECESKYGPPCPPCPAPEFLGGPSVFLFPPKPKDTLMISRTPEVTCVVV | |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVCTLPPSQEEMTKNQVSLSCA | |
| | VKGFYPSDIAVEWESNGQPENNYKTTPPVLDSDGSFFLVSRLTVDKSRWQEG | |
| | NVFSCSVMHEALHNHYTQKSLSLSLGGGGGSRNLPVATPDPGMFPCLHHSQN | |
| | LLRAVSNMLQKARQTLEFYPCTSEEIDHEDITKDKTSTVEACLPLELTKNES | |
| | SLNSRETSFITNGSCLASRKTSFMMALCLSSIYEDLKMYQVEFKTMNAKLLM | |
| | DPKRQIFLDQNMLAVIDELMQALNFNSETVPQKSSLEEPDFYKTKIKLCILL | |
| | HAFRIRAVTIDRVMSYLNAS | |

P37643717

| | | |
|---|---|---|
| Chain 1 | QVQLVQSGSELKKPGASVKVSCKASGYTFTNYDINWVRQAPGQGLEWMGWIF | 175 |
| | PGDESTQYNEKFKGRFVFSLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKESKYGPPCPPCPAPEFLGGPSVFLFPPKPKDTLMISRT | |
| | PEVTCVVVDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPPCQEEMTK | |
| | NQVSLWCLVKGFYPSDIAVEWESNGQPENNYKTTPPVLDSDGSFFLYSRLTV | |
| | DKSRWQEGNVFSCSVMHEALHNHYTQKSLSLSLGGGGGSIWELKKDVYVVEL | |
| | DWYPDAPGEMVVLTCDTPEEDGITWTLDQSSEVLGSGKTLTIQVKEFGDAGQ | |
| | YTCHKGGEVLSHSLLLLHKKEDGIWSTDILKDQKEPKNKTFLRCEAKNYSGR | |
| | FTCWWLTTISTDLTFSVKSSRGSSDPQGVTCGAATLSAERVRGDNKEYEYSV | |
| | ECQEDSAAPAAEESLPIEVMVDAVHKLKYENYTSSFFIRDIIKPDPPKNLQL | |
| | KPLKNSRQVEVSWEYPDTWSTPHSYFSLTFSVQVQGKSKREKKDRVFTDKTS | |
| | ATVICRKNASISVRAQDRYYSSSWSEWASVPCS | |
| Chain 2 | DVVMTQSPAFLSVTPGEKVTITCRASQSISDYLYWYQQKPDQAPKLLIKYAS | 176 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECESKYGPPCPPCPAPEFLGGPSVFLFPPKPKDTLMISRTPEVTCVVV | |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVCTLPPSQEEMTKNQVSLSCA | |
| | VKGFYPSDIAVEWESNGQPENNYKTTPPVLDSDGSFFLVSRLTVDKSRWQEG | |
| | NVFSCSVMHEALHNHYTQKSLSLSLGGGGGSRNLPVATPDPGMFPCLHHSQN | |
| | LLRAVSNMLQKARQTLEFYPCTSEEIDHEDITKDKTSTVEACLPLELTKNES | |
| | SLNSRETSFITNGSCLASRKTSFMMALCLSSIYEDLKMYQVEFKTMNAKLLM | |
| | DPKRQIFLDQNMLAVIDELMQALNFNSETVPQKSSLEEPDFYKTKIKLCILL | |
| | HAFRIRAVTIDRVMSYLNAS | |

P38143816

| | | |
|---|---|---|
| Chain 1 | QVQLVQSGSELKKPGASVKVSCKASGYTFTNYDINWVRQAPGQGLEWMGWIF | 177 |
| | PGDE STQYNEKFKGRFVFSLDTSVSTAYLQISSLKAEDTAVYYCARQTTA | |
| | TWFAYWGQGTLVTVSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKP | |
| | SNTKVDKKVEPKSCDKGGSPLGLAGLSGRSDNRGGGGSIWELKKDVYVVELD | |
| | WYPDAPGEMVVLTCDTPEEDGITWTLDQSSEVLGSGKTLTIQVKEFGDAGQY | |
| | TCHKGGEVLSHSLLLLHKKEDGIWSTDILKDQKEPKNKTFLRCEAKNYSGRF | |
| | TCWWLTTISTDLTFSVKSSRGSSDPQGVTCGAATLSAERVRGDNKEYEYSVE | |
| | CQEDSAAPAAEESLPIEVMVDAVHKLKYENYTSSFFIRDIIKPDPPKNLQLK | |
| | PLKNSRQVEVSWEYPDTWSTPHSYFSLTFSVQVQGQDQDEKKDRVFTDKTSA | |
| | TVICRKNASISVRAQDRYYSSSWSEWASVPCS | |
| Chain 2 | DVVMTQSPAFLSVTPGEKVTITCRASQSISDYLHWYQQKPDQAPKLLIKYAS | 178 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSGGGGSGGGGSRNLPVATPDPGMFPCLHHSQNLLRAVSNMLQ | |
| | KARQTLEFYPCTSEEIDHEDITKDKTSTVEACLPLELTKNESSLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P38153816

| | | |
|---|---|---|
| Chain 1 | QVQLVQSGSELKKPGASVKVSCKASGYTFTNYDINWVRQAPGQGLEWMGWIF | 179 |
| | PGDESTQYNEKFKGRFVFSLDTSVSTAYLQISSLKAEDTAVYYCARQTTATW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGGGSGGGGSGGGGSGGGGSGGGGSIWELKKDVYVVEL | |
| | DWYPDAPGEMVVLTCDTPEEDGITWTLDQSSEVLGSGKTLTIQVKEFGDAGQ | |
| | YTCHKGGEVLSHSLLLLHKKEDGIWSTDILKDQKEPKNKTFLRCEAKNYSGR | |
| | FTCWWLTTISTDLTFSVKSSRGSSDPQGVTCGAATLSAERVRGDNKEYEYSV | |
| | ECQEDSAAPAAEESLPIEVMVDAVHKLKYENYTSSFFIRDIIKPDPPKNLQL | |
| | KPLKNSRQVEVSWEYPDTWSTPHSYFSLTFSVQVQGQDQDEKKDRVFTDKTS | |
| | ATVICRKNASISVRAQDRYYSSSWSEWASVPCS | |
| Chain 2 | DVVMTQSPAFLSVTPGEKVTITCRASQSISDYLHWYQQKPDQAPKLLIKYAS | 180 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSGGGGSGGGGSRNLPVATPDPGMFPCLHHSQNLLRAVSNMLQ | |
| | KARQTLEFYPCTSEEIDHEDITKDKTSTVEACLPLELTKNESSLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P38173819

| | | |
|---|---|---|
| Chain 1 | QVQLVQSGSELKKPGASVKVSCKASGYTFTNYDINWVRQAPGQGLEWMGWIF | 181 |
| | PGDESTQYNEKFKGRFVFSLDTSVSTAYLQISSLKAEDTAVYYCARQTTATW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGSPLGLAGLSGRSDNRGGGGSIWELKKDVYVVELDWY | |
| | PDAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVECQ | |
| | EDSACPAAEESLPIEVMVDAVHKLKYENYTSSFFIRDIIKPDPPKNLQLKPL | |
| | KNSRQVEVSWEYPDTWSTPHSYFSLTFSVQVQGKSKREKKDRVFTDKTSATV | |
| | ICRKNASISVRAQDRYYSSSWSEWASVPCS | |
| Chain 2 | DVVMTQSPAFLSVTPGEKVTITCRASQSISDYLHWYQQKPDQAPKLLIKYAS | 182 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSGGGGSGGGGSRNLPVATPDPGMFPCLHHSQNLLRAVSNMLQ | |
| | KARQTLEFYPCTSEEIDHEDITKDKTSTVEACLPLELTKNESCLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P38183819

| | | |
|---|---|---|
| Chain 1 | QVQLVQSGSELKKPGASVKVSCKASGYTFTNYDINWVRQAPGQGLEWMGWIF | 183 |
| | PGDESTQYNEKFKGRFVFSLDTSVSTAYLQISSLKAEDTAVYYCARQTTATW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGGGSGGGGSGGGGSGGGGSGGGGSIWELKKDVYVVEL | |
| | DWYPDAPGEMVVLTCDTPEEDGITWTLDQSSEVLGSGKTLTIQVKEFGDAGQ | |
| | YTCHKGGEVLSHSLLLLHKKEDGIWSTDILKDQKEPKNKTFLRCEAKNYSGR | |
| | FTCWWLTTISTDLTFSVKSSRGSSDPQGVTCGAATLSAERVRGDNKEYEYSV | |
| | ECQEDSACPAAEESLPIEVMVDAVHKLKYENYTSSFFIRDIIKPDPPKNLQL | |
| | KPLKNSRQVEVSWEYPDTWSTPHSYFSLTFSVQVQGKSKREKKDRVFTDKTS | |
| | ATVICRKNASISVRAQDRYYSSSWSEWASVPCS | |
| Chain 2 | DVVMTQSPAFLSVTPGEKVTITCRASQSISDYLHWYQQKPDQAPKLLIKYAS | 184 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSGGGGSGGGGSRNLPVATPDPGMFPCLHHSQNLLRAVSNMLQ | |
| | KARQTLEFYPCTSEEIDHEDITKDKTSTVEACLPLELTKNESCLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P39043906

| | | |
|---|---|---|
| Chain 1 | QVQLVQSGAEVKKPGASVKVSCKASGYTFTMNIIHWVRQAPGQGLEWMGWIH | 185 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SACPAAEESLPIEVMVDAVHKLKYENYTSSFFIRDIIKPDPPKNLQLKPLKN | |
| | SRQVEVSWEYPDTWSTPHSYFSLTFSVQVQGQDQDEKKDRVFTDKTSATVIC | |
| | RKNASISVRAQDRYYSSSWSEWASVPCS | |
| Chain 2 | EIVLTQSPGTLSLSPGERATLSCRASQSVSTSAYSYMHWYQQKPGQAPRLLI | 186 |
| | YLASNLESGIPDRFSGSGSGTDFTLTISRLEPEDFAVYYCQQSRELPYTEGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGECGGGGSPLGLAGRNLPVATPDPGMFPCLHHSQNLLRAVSNMLQ | |
| | KARQTLEFYPCTSEEIDHEDITKDKTSTVEACLPLELTKNESCLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P39043907

| | | |
|---|---|---|
| Chain 1 | QVQLVQSGAEVKKPGASVKVSCKASGYTFTMNIIHWVRQAPGQGLEWMGWIH | 187 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SACPAAEESLPIEVMVDAVHKLKYENYTSSFFIRDIIKPDPPKNLQLKPLKN | |
| | SRQVEVSWEYPDTWSTPHSYFSLTFSVQVQGQDQDEKKDRVFTDKTSATVIC | |
| | RKNASISVRAQDRYYSSSWSEWASVPCS | |
| Chain 2 | EVQLVESGGGLVQPGNSLRLSCAASGFTFSSFGMSWVRQAPGKGLEWVSSIS | 188 |
| | GSGSDTLYADSVKGRFTISRDNAKTTLYLQMNSLRPEDTAVYYCTIGGSLSR | |
| | SSQGTLVTVSSEIVLTQSPGTLSLSPGERATLSCRASQSVSTSAYSYMHWYQ | |
| | QKPGQAPRLLIYLASNLESGIPDRFSGSGSGTDFTLTISRLEPEDFAVYYCQ | |
| | QSRELPYTFGQGTKLEIKRTVAAPSVFIFPPSDEQLKSGTASVVCLLNNFYP | |
| | REAKVQWKVDNALQSGNSQESVTEQDSKDSTYSLSSTLTLSKADYEKHKVYA | |
| | CEVTHQGLSSPVTKSFNRGECGGGGSPLGLAGRNLPVATPDPGMFPCLHHSQ | |
| | NLLRAVSNMLQKARQTLEFYPCTSEEIDHEDITKDKTSTVEACLPLELTKNE | |
| | SCLNSRETSFITNGSCLASRKTSFMMALCLSSIYEDLKMYQVEFKTMNAKLL | |
| | MDPKRQIFLDQNMLAVIDELMQALNFNSETVPQKSSLEEPDFYKTKIKLCIL | |
| | LHAFRIRAVTIDRVMSYLNAS | |

P39053906

| | | |
|---|---|---|
| Chain 1 | QVQLVQSGAEVKKPGASVKVSCKASGYTFTMNIIHWVRQAPGQGLEWMGWIH | 189 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SACPAAEESLPIEVMVDAVHKLKYENYTSSFFIRDIIKPDPPKNLQLKPLKN | |
| | SRQVEVSWEYPDTWSTPHSYFSLTFSVQVQGKSKREKKDRVFTDKTSATVIC | |
| | RKNASISVRAQDRYYSSSWSEWASVPCS | |
| Chain 2 | EIVLTQSPGTLSLSPGERATLSCRASQSVSTSAYSYMHWYQQKPGQAPRLLI | 190 |
| | YLASNLESGIPDRESGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGECGGGGSPLGLAGRNLPVATPDPGMFPCLHHSQNLLRAVSNMLQ | |
| | KARQTLEFYPCTSEEIDHEDITKDKTSTVEACLPLELTKNESCLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P39053907

| | | |
|---|---|---|
| Chain 1 | QVQLVQSGAEVKKPGASVKVSCKASGYTFTMNIIHWVRQAPGQGLEWMGWIH | 191 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SACPAAEESLPIEVMVDAVHKLKYENYTSSFFIRDIIKPDPPKNLQLKPLKN | |
| | SRQVEVSWEYPDTWSTPHSYFSLTFSVQVQGKSKREKKDRVFTDKTSATVIC | |
| | RKNASISVRAQDRYYSSSWSEWASVPCS | |
| Chain 2 | EVQLVESGGGLVQPGNSLRLSCAASGFTFSSFGMSWVRQAPGKGLEWVSSIS | 192 |
| | GSGSDTLYADSVKGRFTISRDNAKTTLYLQMNSLRPEDTAVYYCTIGGSLSR | |
| | SSQGTLVTVSSEIVLTQSPGTLSLSPGERATLSCRASQSVSTSAYSYMHWYQ | |
| | QKPGQAPRLLIYLASNLESGIPDRFSGSGSGTDFTLTISRLEPEDFAVYYCQ | |
| | QSRELPYTFGQGTKLEIKRTVAAPSVFIFPPSDEQLKSGTASVVCLLNNFYP | |
| | REAKVQWKVDNALQSGNSQESVTEQDSKDSTYSLSSTLTLSKADYEKHKVYA | |
| | CEVTHQGLSSPVTKSFNRGECGGGGSPLGLAGRNLPVATPDPGMFPCLHHSQ | |
| | NLLRAVSNMLQKARQTLEFYPCTSEEIDHEDITKDKTSTVEACLPLELTKNE | |
| | SCLNSRETSFITNGSCLASRKTSFMMALCLSSIYEDLKMYQVEFKTMNAKLL | |
| | MDPKRQIFLDQNMLAVIDELMQALNFNSETVPQKSSLEEPDFYKTKIKLCIL | |
| | LHAFRIRAVTIDRVMSYLNAS | |

P40453671

| | | |
|---|---|---|
| Chain 1 | QVQLVQSGAEVKKPGASVKVSCKASGYTFTMNIIHWVRQAPGQGLEWMGWIH | 193 |
| | PGSGAIKYAQKLQGRVTMTTDTSTSTAYMELRSLRSDDTAVYYCARHGGTGR | |
| | GAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKGGGGSSGRSDNRGGGGSIWELKKDVYVVELDWYPD | |
| | APGEMVVLTCDTPEEDGITWTLDQSSEVLGSGKTLTIQVKEFGDAGQYTCHK | |
| | GGEVLSHSLLLLHKKEDGIWSTDILKDQKEPKNKTFLRCEAKNYSGRFTCWW | |
| | LTTISTDLTFSVKSSRGSSDPQGVTCGAATLSAERVRGDNKEYEYSVECQED | |
| | SAAPAAEESLPIEVMVDAVHKLKYENYTSSFFIRDIIKPDPPKNLQLKPLKN | |
| | SRQVEVSWEYPDTWSTPHSYFSLTFSVQVQGQDQDEKKDRVFTDKTSATVIC | |
| | RKNASISVRAQDRYYSSSWSEWASVPCSGGGGSGGGGSDAHKSEVAHRFKDL | |
| | GEENFKALVLIAFAQYLQQCPFEDHVKLVNEVTEFAKTCVADESAENCDKSL | |
| | HTLFGDKLCTVATLRETYGEMADCCAKQEPERNECFLQHKDDNPNLPRLVRP | |
| | EVDVMCTAFHDNEETFLKKYLYEIARRHPYFYAPELLFFAKRYKAAFTECCQ | |
| | AADKAACLLPKLDELRDEGKASSAKQRLKCASLQKFGERAFKAWAVARLSQR | |
| | FPKAEFAEVSKLVTDLTKVHTECCHGDLLECADDRADLAKYICENQDSISSK | |
| | LKECCEKPLLEKSHCIAEVENDEMPADLPSLAADFVESKDVCKNYAEAKDVE | |
| | LGMFLYEYARRHPDYSVVLLLRLAKTYETTLEKCCAAADPHECYAKVEDEFK | |
| | PLVEEPQNLIKQNCELFEQLGEYKFQNALLVRYTKKVPQVSTPTLVEVSRNL | |
| | GKVGSKCCKHPEAKRMPCAEDYLSVVLNQLCVLHEKTPVSDRVTKCCTESLV | |
| | NRRPCFSALEVDETYVPKEFNAETFTFHADICTLSEKERQIKKQTALVELVK | |
| | HKPKATKEQLKAVMDDFAAFVEKCCKADDKETCFAEEGKKLVAASQAALGL | |
| Chain 2 | EIVLTQSPGTLSLSPGERATLSCRASQSVSTSAYSYMHWYQQKPGQAPRLLI | 194 |
| | YLASNLESGIPDRFSGSGSGTDFTLTISRLEPEDFAVYYCQQSRELPYTFGQ | |
| | GTKLEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGECGGGGSPLGLAGRNLPVATPDPGMFPCLHHSQNLLRAVSNMLQ | |
| | KARQTLEFYPCTSEEIDHEDITKDKTSTVEACLPLELTKNESSLNSRETSFI | |
| | TNGSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAVTI | |
| | DRVMSYLNAS | |

P40463712

| | | |
|---|---|---|
| Chain 1 | QVQLVQSGAEVKKPGSSVKVSCKTSGDTFSTYAISWVRQAPGQGLEWMGGII | 195 |
| | PIFGKAHYAQKFQGRVTITADESTSTAYMELSSLRSEDTAVYFCARKFHFVS | |
| | GSPFGMDVWGQGTTVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNV | |
| | NHKPSNTKVDKKVEPKSCDKGGSPLGLAGLSGRSDNRGGGGSIWELKKDVYV | |
| | VELDWYPDAPGEMVVLTCDTPEEDGITWTLDQSSEVLGSGKTLTIQVKEFGD | |
| | AGQYTCHKGGEVLSHSLLLLHKKEDGIWSTDILKDQKEPKNKTFLRCEAKNY | |
| | SGRFTCWWLTTISTDLTFSVKSSRGSSDPQGVTCGAATLSAERVRGDNKEYE | |
| | YSVECQEDSAAPAAEESLPIEVMVDAVHKLKYENYTSSFFIRDIIKPDPPKN | |
| | LQLKPLKNSRQVEVSWEYPDTWSTPHSYFSLTFSVQVQGQDQDEKKDRVFTD | |
| | KTSATVICRKNASISVRAQDRYYSSSWSEWASVPCSGGGGSGGGGSDAHKSE | |
| | VAHRFKDLGEENFKALVLIAFAQYLQQCPFEDHVKLVNEVTEFAKTCVADES | |
| | AENCDKSLHTLFGDKLCTVATLRETYGEMADCCAKQEPERNECFLQHKDDNP | |
| | NLPRLVRPEVDVMCTAFHDNEETFLKKYLYEIARRHPYFYAPELLFFAKRYK | |
| | AAFTECCQAADKAACLLPKLDELRDEGKASSAKQRLKCASLQKFGERAFKAW | |
| | AVARLSQRFPKAEFAEVSKLVTDLTKVHTECCHGDLLECADDRADLAKYICE | |
| | NQDSISSKLKECCEKPLLEKSHCIAEVENDEMPADLPSLAADFVESKDVCKN | |
| | YAEAKDVFLGMFLYEYARRHPDYSVVLLLRLAKTYETTLEKCCAAADPHECY | |
| | AKVFDEFKPLVEEPQNLIKQNCELFEQLGEYKFQNALLVRYTKKVPQVSTPT | |
| | LVEVSRNLGKVGSKCCKHPEAKRMPCAEDYLSVVLNQLCVLHEKTPVSDRVT | |
| | KCCTESLVNRRPCFSALEVDETYVPKEFNAETFTFHADICTLSEKERQIKKQ | |
| | TALVELVKHKPKATKEQLKAVMDDFAAFVEKCCKADDKETCFAEEGKKLVAA | |
| | SQAALGL | |
| Chain 2 | EIVLTQSPATLSLSPGERATLSCRASQSVSSYLAWYQQKPGQAPRLLIYDAS | 196 |
| | NRATGIPARFSGSGSGTDFTLTISSLEPEDFAVYYCQQRSNWPTFGQGTKVE | |
| | IKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGECGGGGSRNLPVATPDPGMFPCLHHSQNLLRAVSNMLQKARQTLEFYPC | |
| | TSEEIDHEDITKDKTSTVEACLPLELTKNESSLNSRETSFITNGSCLASRKT | |
| | SFMMALCLSSIYEDLKMYQVEFKTMNAKLLMDPKRQIFLDQNMLAVIDELMQ | |
| | ALNENSETVPQKSSLEEPDFYKTKIKLCILLHAFRIRAVTIDRVMSYLNAS | |

P40473715

| | | |
|---|---|---|
| Chain 1 | QVQLVQSGSELKKPGASVKVSCKASGYTFTNYDINWVRQAPGQGLEWMGWIF | 197 |
| | PGDESTQYNEKFKGRFVESLDTSVSTAYLQISSLKAEDTAVYYCARQTTGTW | |
| | FAYWGQGTLVTVSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKGGSPLGLAGLSGRSDNRGGGGSIWELKKDVYVVELDWY | |
| | PDAPGEMVVLTCDTPEEDGITWTLDQSSEVLGSGKTLTIQVKEFGDAGQYTC | |
| | HKGGEVLSHSLLLLHKKEDGIWSTDILKDQKEPKNKTFLRCEAKNYSGRFTC | |
| | WWLTTISTDLTFSVKSSRGSSDPQGVTCGAATLSAERVRGDNKEYEYSVECQ | |
| | EDSAAPAAEESLPIEVMVDAVHKLKYENYTSSFFIRDIIKPDPPKNLQLKPL | |
| | KNSRQVEVSWEYPDTWSTPHSYFSLTFSVQVQGQDQDEKKDRVFTDKTSATV | |
| | ICRKNASISVRAQDRYYSSSWSEWASVPCSGGGGSGGGGSDAHKSEVAHRFK | |
| | DLGEENFKALVLIAFAQYLQQCPFEDHVKLVNEVTEFAKTCVADESAENCDK | |
| | SLHTLFGDKLCTVATLRETYGEMADCCAKQEPERNECFLQHKDDNPNLPRLV | |
| | RPEVDVMCTAFHDNEETFLKKYLYEIARRHPYFYAPELLFFAKRYKAAFTEC | |
| | CQAADKAACLLPKLDELRDEGKASSAKQRLKCASLQKFGERAFKAWAVARLS | |
| | QRFPKAEFAEVSKLVTDLTKVHTECCHGDLLECADDRADLAKYICENQDSIS | |
| | SKLKECCEKPLLEKSHCIAEVENDEMPADLPSLAADFVESKDVCKNYAEAKD | |
| | VFLGMFLYEYARRHPDYSVVLLLRLAKTYETTLEKCCAAADPHECYAKVEDE | |
| | FKPLVEEPQNLIKQNCELFEQLGEYKFQNALLVRYTKKVPQVSTPTLVEVSR | |
| | NLGKVGSKCCKHPEAKRMPCAEDYLSVVLNQLCVLHEKTPVSDRVTKCCTES | |
| | LVNRRPCFSALEVDETYVPKEFNAETFTFHADICTLSEKERQIKKQTALVEL | |
| | VKHKPKATKEQLKAVMDDFAAFVEKCCKADDKETCFAEEGKKLVAASQAALG | |
| | L | |
| Chain 2 | DVVMTQSPAFLSVTPGEKVTITCRASQSISDYLYWYQQKPDQAPKLLIKYAS | 198 |
| | QSISGIPARFSGSGSGTDFTFTISSLEAEDAATYYCQNGHSFPLTFGQGTKL | |
| | ELKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGECGGGGSRNLPVATPDPGMFPCLHHSQNLLRAVSNMLQKARQTLEFYP | |
| | CTSEEIDHEDITKDKTSTVEACLPLELTKNESSLNSRETSFITNGSCLASRK | |
| | TSFMMALCLSSIYEDLKMYQVEFKTMNAKLLMDPKRQIFLDQNMLAVIDELM | |
| | QALNFNSETVPQKSSLEEPDFYKTKIKLCILLHAFRIRAVTIDRVMSYLNAS | |

Thus, in certain embodiments, the first polypeptide (or chain 1) comprises, consists, or consists essentially of an amino acid sequence that is at least 80, 85, 90, 95, 98, or 100% identical to a sequence selected from Table S4, and the second polypeptide (or chain 2) comprises, consists, or consists essentially of a corresponding amino acid sequence that is at least 80, 85, 90, 95, 98, or 100% identical to a sequence selected from Table S4. In certain embodiments, as indicated, the heterodimer comprises, consists, or consists essentially of a chain 3 amino acid sequence that is at least 80, 85, 90, 95, 98, or 100% identical to a chain 3 sequence selected from Table S4.

Human IL-12A (p35) Protein Variants

Certain embodiments include an isolated human IL-12A (p35) protein variant, which comprises, consists, or consists essentially of an amino acid sequence that is at least 80, 85, 90, 95, 98, or 100% identical to the amino acid sequence set forth in SEQ ID NO: 1 or 2, and which has an amino acid substitution at any one or more of E38, F39, P41, C74, K128, F166, Y167, S197, and/or D188, as defined by the mature p35 sequence. In some embodiments, the isolated human p35 protein variant has an amino acid substitution selected from any one or more of:
E38A, E38D, E38F, E38G, E38H, E38I, E38K, E38L, E38M, E38N, E38P, E38Q, E38R, E38S, E38T, E38V, or E38W;
F39A, F39D, F39E, F39G, F39H, F39I, F39K, F39L, F39M, F39N, F39P, F39Q, F39R, F39S, F39T, F39V, or F39W;
P41A, P41D, P41E, P41F, P41G, P41H, P41I, P41K, P41L, P41M, P41N, P41Q, P41R, P41S, P41T, P41V, or P41W;
C74A or C74S;
K128A, K128D, K128E, K128F, K128G, K128H, K128I, K128L, K128M, K128N, K128P, K128Q, K128R, K128S, K128T, K128V, or K128W;
F166A, F166D, F166E, F166G, F166H, F166I, F166K, F166L, F166M, F166N, F166P, F166Q, F166R, F166S, F166T, F166V, or F166W
S197A; and
D188A, including any combination of the foregoing. In some embodiments, the isolated p35 protein variant has reduced binding affinity to wild-type IL-12Rβ31/IL-12Rβ2 receptor complex of about or at least about 2-fold, 5-fold, 10-fold, 50-fold, 100-fold, 1000-fold or more, relative to the binding affinity of the wild-type p35 sequence.

The p35 protein variants can used, for example, as standalone, isolated proteins, as part of IL-12 complexes in combination with IL-12B(p40) proteins, or as part of fusion proteins or other complexes. The p35 protein variants can be used in any of the methods or compositions described herein, including any one or more of the attenuated protein homodimers or heterodimers described herein.

### Methods of Use and Pharmaceutical Compositions

Certain embodiments include methods of treating, ameliorating the symptoms of, and/or reducing the progression of, a disease or condition in a subject in need thereof, comprising administering to the subject at least one attenuated protein homodimer or heterodimer, as described herein, or an IL-12A (p35) protein variant, as described herein. Also included are methods of enhancing an immune response in a subject comprising administering to the subject at least one attenuated protein homodimer or heterodimer, as described herein, or an IL-12A protein variant, as described herein. In particular embodiments, the disease is selected from one or more of a cancer, a viral infection, and an immune disorder.

In some embodiments, following administration, the attenuated protein homodimer or heterodimer is “activated” through binding of the ABD to the cell surface protein, plasma protein, or ECM protein, which increases binding (avidity) of the IL-12 protein(s) in the homodimer or heterodimer to the IL-12Rβ1/IL-12Rβ2 receptor complex on the surface of the immune cell in vitro or in vivo, and thereby increases at least one IL-12 signaling activity of the homodimer or heterodimer. In particular embodiments, the activation occurs in a cancer cell or cancer tissue, or a virally-infected cell or virally-infected tissue.

Typically, the activated homodimer or heterodimer protein has at least one immune-stimulating IL-12 signaling activity, for example, by binding to the IL-12Rβ1/IL-12Rβ2 receptor complex on the surface of an immune cell in vivo, and thereby stimulating the immune cell. Exemplary IL-12 signaling activities include stimulating growth and function of T cells, enhancing cytotoxic activity of NK cells and/or CD8+ T cells, stimulating production of interferon-γ (IFN-γ) and/or tumor necrosis factor-α (TNF-α), and inhibiting angiogenesis.

In particular embodiments, the immune cell is selected from one or more of a T cell, a B cell, a natural killer cell, a monocyte, and a macrophage. In some embodiments, the immune cell is an exhausted T cell or an exhausted NK cell. An “exhausted” cell refers to an immune cell that is immunologically dysfunctional despite repeated exposure to antigens. In some instances, “exhaustion” is defined by poor effector function, sustained expression of inhibitory receptors, and for T cells a transcriptional state distinct from that of functional effector or memory T cells (see, for example, Wherry, Nature Immunol. 12:492-499, 2011; and Beltra et al., Immunity. 52:825-841, 2020). Certain exhausted T cells co-express multiple co-inhibitory receptors (for example, PD-1, CTLA-4, LAG-3, TIGIT, CD39, TIM-3), which dampen T-cell activation by various mechanisms.

In some embodiments, administration and activation of the attenuated protein homodimer or heterodimer increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some instances, the immune response is an anti-cancer or anti-viral immune response. In some embodiments, administration and activation of the attenuated protein homodimer or heterodimer increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control. In some embodiments, wherein the cell-killing is cancer cell-killing or virally-infected cell-killing.

In some embodiments, the disease is a cancer, that is, the subject in need thereof has or is suspected of having a cancer. Certain embodiments thus include methods of treating, ameliorating the symptoms of, or inhibiting the progression of, a cancer in a subject in need thereof, comprising administering to the subject at least one attenuated protein homodimer or heterodimer, as described herein. In particular embodiments, the cancer is a primary cancer or a metastatic cancer. In specific embodiments, the cancer is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject at least one attenuated protein homodimer or heterodimer, as described herein, in combination with at least one additional agent, for example, a chemotherapeutic agent, a hormonal therapeutic agent, and/or a kinase inhibitor. In some embodiments, administering the at least one attenuated protein homodimer or heterodimer enhances the susceptibility of the cancer to the additional agent (for example, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), an anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine); Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The various chemotherapeutic agents described herein can be combined with any one or more of the attenuated protein homodimers or heterodimers described herein, and used according to any one or more of the methods or compositions described herein.

Certain combination therapies employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The various hormonal therapeutic agents described herein can be combined with any one or more of the various attenuated protein homodimers or heterodimers described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The various kinase inhibitors described herein can be combined with any one or more of the various attenuated protein homodimers or heterodimers described herein, and used according to any one or more of the methods or compositions described herein.

In some embodiments, the methods and pharmaceutical compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and pharmaceutical compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease.

In some embodiments, the disease is a viral disease or viral infection. In certain embodiments, the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis. In specific embodiments, the subject is HIV-positive. In some embodiments, the methods and pharmaceutical compositions described herein increase an anti-viral immune response by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the immune disorder is selected from one or more of type 1 diabetes, vasculitis, and an immunodeficiency. In some embodiments, the methods and pharmaceutical compositions described herein improve immune function in the subject, for example, by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control.

In certain embodiments, the methods and therapeutic compositions described herein are sufficient to result in clinically relevant reduction in symptoms of a particular disease indication known to the skilled clinician.

For in vivo use, as noted above, for the treatment of human or non-human mammalian disease or testing, the agents described herein are generally incorporated into one or more therapeutic or pharmaceutical compositions prior to administration, including veterinary therapeutic compositions.

Thus, certain embodiments relate to pharmaceutical or therapeutic compositions that comprise at least one attenuated protein homodimer or heterodimer, as described herein, or an IL-12A (p35) protein variant, as described herein. In some instances, a pharmaceutical or therapeutic composition comprises one or more of the attenuated protein homodimers or heterodimers described herein, or an IL-12A (p35) protein variant, as described herein, in combination with a pharmaceutically- or physiologically-acceptable carrier or excipient. Certain pharmaceutical or therapeutic compositions further comprise at least one additional agent, for example, a chemotherapeutic agent, a hormonal therapeutic agent, and/or a kinase inhibitor as described herein.

Some therapeutic compositions comprise (and certain methods utilize) only one attenuated protein homodimer or heterodimer or IL-12A protein variant. Certain therapeutic compositions comprise (and certain methods utilize) a mixture of at least two, three, four, or five different attenuated protein homodimers, heterodimers, and/or IL-12A (p35) protein variants, as described herein.

In particular embodiments, the pharmaceutical or therapeutic compositions comprising at least one protein described herein is substantially pure on a protein basis or a weight-weight basis, for example, the composition has a purity of at least about 80%, 85%, 90%, 95%, 98%, or 99% on a protein basis or a weight-weight basis.

In some embodiments, the proteins described herein do not form aggregates, have a desired solubility, and/or have an immunogenicity profile that is suitable for use in humans, as known in the art. Thus, in some embodiments, the therapeutic composition comprising a protein described herein is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise a protein that is at least about 50%, about 60%, about 70%, about 80%, about 90% or about 95% monodisperse with respect to its apparent molecular mass.

In some embodiments, the proteins described herein are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains an attenuated protein homodimer or heterodimer and an additional therapeutic agent (e.g., chemotherapeutic agent, hormonal therapeutic agent, kinase inhibitor), as well as administration of compositions comprising an attenuated protein homodimer or heterodimer and an additional therapeutic agent in its own separate pharmaceutical dosage formulation. For example, an attenuated protein homodimer or heterodimer and an additional therapeutic agent can be administered to the subject together in a single oral dosage composition such as a tablet or capsule, or each agent administered in separate oral dosage formulations. Similarly, an attenuated protein homodimer or heterodimer and an additional therapeutic agent can be administered to the subject together in a single parenteral dosage composition such as in a saline solution or other physiologically acceptable solution, or each agent administered in separate parenteral dosage formulations. As another example, for cell-based therapies, an attenuated protein homodimer or heterodimer can be mixed with the cells prior to administration, administered as part of a separate composition, or both. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one attenuated protein homodimer or heterodimer, as described herein; and optionally (b) at least one additional therapeutic agent (e.g., chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an attenuated protein homodimer or heterodimer and optionally at least one additional therapeutic agent. For example, the kit includes a plurality of syringes, ampules, foil packets, or blister packs, each containing a single unit dose of an attenuated protein homodimer or heterodimer and optionally at least one additional therapeutic agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

Expression and Purification Systems

Certain embodiments include methods and related compositions for expressing and purifying the protein components of an attenuated protein homodimer or heterodimer, as described herein, or an IL-12A (p35) protein variant, as described herein. Such recombinant proteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, may be prepared by a procedure including one or more of the steps of: (a) preparing one or more vectors or constructs comprising one or more polynucleotide sequences that encode a first polypeptide (and optionally a second polypeptide, or additional polypeptides, for example, a region of an ABD) of a homodimer or heterodimer, which are operably linked to one or more regulatory elements; (b) introducing the one or more vectors or constructs into one or more host cells; (c) culturing the one or more host cell to express the polypeptides, which bind together to form an attenuated protein homodimer or heterodimer; and (d) isolating the attenuated protein homodimer or heterodimer from the host cell. Alternatively, especially for heterodimers, the first and second polypeptides, or any additional polypeptides (e.g., a region of an ABD), can be produced in separate host cells, isolated separately, and then combined to form an attenuated protein heterodimer.

To express a desired polypeptide, a nucleotide sequence encoding one or more proteins of interest may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector-enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in lon and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS•TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5:Unit 5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grown on serum free medium (see, e.g., Rosser et al., Protein Expr. Purif. 40:237-43, 2005; and U.S. Pat. No. 6,210,922).

A protein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6×His), cMyc tags, V5-tags, glutathione S-transferase (GST) tags, and others.

A protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., Coomassie, silver stain), immunoblot, Bradford, and ELISA, which may be utilized during any step of the production or purification process, typically to measure the purity of the protein composition.

Also included are methods of concentrating proteins, and compositions comprising concentrated soluble proteins, e.g., attenuated protein homodimers or heterodimers. In some aspects, such concentrated solutions of at least one protein comprise proteins at a concentration of about or at least about 5 mg/mL, 8 mg/mL, 10 mg/mL, 15 mg/mL, 20 mg/mL, or more.

In some aspects, such compositions are substantially monodisperse, meaning that a protein exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, a protein composition has a purity of at least about 90%, as measured according to routine techniques in the art. In certain embodiments, such as diagnostic compositions or certain pharmaceutical or therapeutic compositions, a protein composition has a purity of at least about 95%, or at least about 97% or 98% or 99%. In some embodiments, such as when being used as reference or research reagents, a protein composition can be of lesser purity, and may have a purity of at least about 50%, 60%, 70%, or 80%. Purity can be measured overall or in relation to selected components, such as other proteins, e.g., purity on a protein basis.

Purified proteins can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to cell-based assays, including those that utilize at least one IL-12 receptor or receptor complex, which is optionally functionally coupled to a readout or indicator, such as a fluorescent or luminescent indicator of biological activity, as described herein.

In certain embodiments, as noted above, a composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, a composition is made from a eukaryotic cell such as a mammalian or human cell in substantially serum free media. In certain embodiments, as noted herein, a composition has an endotoxin content of less than about 10 EU/mg of protein, or less than about 5 EU/mg of protein, less than about 3 EU/mg of protein, or less than about 1 EU/mg of protein.

In certain embodiments, a composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of proteins and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Proteins with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains a protein of the present disclosure.

All publications, patent applications, and issued patents cited in this specification are herein incorporated by reference as if each individual publication, patent application, or issued patent were specifically and individually indicated to be incorporated by reference.

### EXAMPLES

### Example 1

### Engineering and Testing of Attenuated IL-12 Homodimers

IL-12 (p35-p40) fusion proteins were generated by fusing wild-type or mutant human p35 to the N-terminus of wild-type or mutant human p40. Table E1 shows the p35 and p40 wt/mutants used in the p35-p40 fusion proteins with a His-tag at the C-terminus of p40.

TABLE E1

| | p35 | | p40 | | | |
|---|---|---|---|---|---|---|
| Protein name | Mutants | | Mutants | | | |
| P3074 | — | — | QDQD (258-261) | — | — | — |
| P3075 | — | — | QDQD (258-261) | — | — | — |
| P3076 | — | — | QDQD (258-261) | — | — | — |
| P3077 | C74S | — | QDQD (258-261) | — | C177A | — |
| P3078 | C74S | — | QDQD (258-261) | — | C177A | — |
| P3079 | C74S | — | QDQD (258-261) | — | C177A | — |
| P3080 | C74S | — | QDQD (258-261) | Y114F | C177A | — |
| P3081 | C74S | — | QDQD (258-261) | Y114F | C177A | — |
| P3082 | C74S | — | QDQD (258-261) | Y114F | C177A | — |
| P3189 | C74S | S197A | QDQD (258-261) | — | C177A | — |
| P3190 | C74S | S197A | QDQD (258-261) | — | C177A | C252S |
| P3191 | C74S | S197A | — | — | C177A | — |
| P3192 | C74S | S197A | — | — | C177A | C252S |
| P3193 | — | S197A | QDQD (258-261) | — | — | — |
| P3194 | — | S197A | QDQD (258-261) | — | — | C252S |
| P3195 | — | S197A | — | — | — | — |
| P3196 | — | S197A | — | — | — | C252S |

IL-12 (p40-p35) fusion proteins were also generated by fusing wild-type or mutant human p40 to the N-terminus of wild-type or mutant human p35. Table E2 shows the p40 and p35 wt/mutants used in the p40-p35 fusion proteins with a His-tag at the C-terminus of p35.

TABLE E2

| | p35 | | p40 | | |
|---|---|---|---|---|---|
| Protein name | Mutants | | Mutants | | |
| P3336 | C74S | — | — | C177A | — |
| P3337 | C74S | — | KSKR --> QDQD | C177A | — |
| P3338 | C74S | S197A | KSKR --> QDQD | C177A | — |
| P3339 | C74S | — | — | C177A | C252S |
| P3340 | C74S | — | KSKR --> QDQD | C177A | C252S |
| P3341 | C74S | S197A | KSKR --> QDQD | C177A | C252S |

Antibody-IL-12 (p35-p40) fusion proteins were generated by fusing an immunoglobulin antigen binding domain (ABD) to the N-terminus of the p35-p40 fusion proteins, as shown in Table E3.

TABLE E3

| | p35 | | | p40 | | |
|---|---|---|---|---|---|---|
| Protein name | ABD Target | Mutants | | Mutants | | |
| P3083 | Non-targeted | C74S | — | QDQD (258-261) | C177A | — |
| P3084 | Non-targeted | C74S | — | QDQD (258-261) | C177A | — |
| P3085 | Non-targeted | C74S | — | QDQD (258-261) | C177A | — |
| P30952158 | FAPa, B7H3 | C74S | — | QDQD (258-261) | C177A | — |
| P30962158 | FAPa, B7H3 | C74S | — | QDQD (258-261) | C177A | — |
| P30972158 | FAPa, B7H3 | C74S | — | QDQD (258-261) | C177A | — |
| P30982158 | FAPa, B7H3 | C74S | — | QDQD (258-261) | C177A | — |
| P30991942 | PD-L1 | C74S | — | QDQD (258-261) | C177A | — |
| P31001942 | PD-L1 | C74S | — | QDQD (258-261) | C177A | — |
| P31011563 | FAPa | C74S | — | QDQD (258-261) | C177A | — |
| P31021563 | FAPa | C74S | — | QDQD (258-261) | C177A | — |
| P32692158 | FAPa, B7H3 | C74S | S197A | QDQD (258-261) | C177A | C252S |
| P32702158 | FAPa, B7H3 | C74S | S197A | QDQD (258-261) | C177A | C252S |
| P32712158 | FAPa, B7H3 | C74S | S197A | QDQD (258-261) | C177A | C252S |
| P32721942 | PD-L1 | C74S | S197A | QDQD (258-261) | C177A | C252S |
| P32731942 | PD-L1 | C74S | S197A | QDQD (258-261) | C177A | C252S |
| P32741942 | PD-L1 | C74S | S197A | QDQD (258-261) | C177A | C252S |

To generate ABD-p35-p40 fusion proteins with attenuated IL-12R binding activities, various point mutations were introduced at position 167 of p35, based on the construct P32731942 from Table E3 (supra). The design of the attenuated constructs is shown in Table E4.

TABLE E4

| | p35 | | | | P40 | | |
|---|---|---|---|---|---|---|---|
| Protein name | ABD Target | Mutants | | | Mutants | | |
| P32771942 | PD-L1 | C74S | S197A | D188A | QDQD (258-261) | C177A | C252S |
| P32781942 | PD-L1 | C74S | S197A | Y167A | QDQD (258-261) | C177A | C252S |
| P32791942 | PD-L1 | C74S | S197A | Y167D | QDQD (258-261) | C177A | C252S |
| P32801942 | PD-L1 | C74S | S197A | Y167E | QDQD (258-261) | C177A | C252S |
| P32811942 | PD-L1 | C74S | S197A | Y167F | QDQD (258-261) | C177A | C252S |
| P32821942 | PD-L1 | C74S | S197A | Y167G | QDQD (258-261) | C177A | C252S |
| P32831942 | PD-L1 | C74S | S197A | Y167H | QDQD (258-261) | C177A | C252S |
| P32841942 | PD-L1 | C74S | S197A | Y167I | QDQD (258-261) | C177A | C252S |
| P32851942 | PD-L1 | C74S | S197A | Y167L | QDQD (258-261) | C177A | C252S |
| P32861942 | PD-L1 | C74S | S197A | Y167N | QDQD (258-261) | C177A | C252S |
| P32871942 | PD-L1 | C74S | S197A | Y167Q | QDQD (258-261) | C177A | C252S |
| P32881942 | PD-L1 | C74S | S197A | Y167S | QDQD (258-261) | C177A | C252S |
| P32891942 | PD-L1 | C74S | S197A | Y167T | QDQD (258-261) | C177A | C252S |
| P32901942 | PD-L1 | C74S | S197A | Y167V | QDQD (258-261) | C177A | C252S |

Additional ABD-p35-p40 fusion proteins were generated by fusing a non-targeting (control) ABD to the N-terminus of the p35-p40 fusion proteins, as shown in Table E5.

| Protein | p35 | | | | |
|---|---|---|---|---|---|
| name | ABD Target | Mutants | | | LC |
| P32731563 | PD-L1 | C74S | S197A | — | M85LC |
| P32771563 | PD-L1 | C74S | S197A | D188A | M85LC |
| P32781563 | PD-L1 | C74S | S197A | Y167A | M85LC |
| P32791563 | PD-L1 | C74S | S197A | Y167D | M85LC |
| P32801563 | PD-L1 | C74S | S197A | Y167E | M85LC |
| P32811563 | PD-L1 | C74S | S197A | Y167F | M85LC |
| P32821563 | PD-L1 | C74S | S197A | Y167G | M85LC |
| P32831563 | PD-L1 | C74S | S197A | Y167H | M85LC |
| P32841563 | PD-L1 | C74S | S197A | Y167I | M85LC |
| P32851563 | PD-L1 | C74S | S197A | Y167L | M85LC |
| P32861563 | PD-L1 | C74S | S197A | Y167N | M85LC |
| P32871563 | PD-L1 | C74S | S197A | Y167Q | M85LC |
| P32881563 | PD-L1 | C74S | S197A | Y167S | M85LC |
| P32891563 | PD-L1 | C74S | S197A | Y167T | M85LC |
| P32901563 | PD-L1 | C74S | S197A | Y167V | M85LC |

Plasmids coding for the fusion proteins were constructed by standard gene synthesis or mutagenesis, and followed by sub-cloning into the pTT5 expression vector.

Production, purification, and characterization. Fusion proteins were produced by transient transfection in Expi293 cells. His-tagged proteins were purified by one-step purification process of nickel affinity chromatography (GE Healthcare). ABD fusion proteins were purified by a one-step purification of CaptureSelect™ IgG-CH1 Affinity Matrix (ThermoFisher).

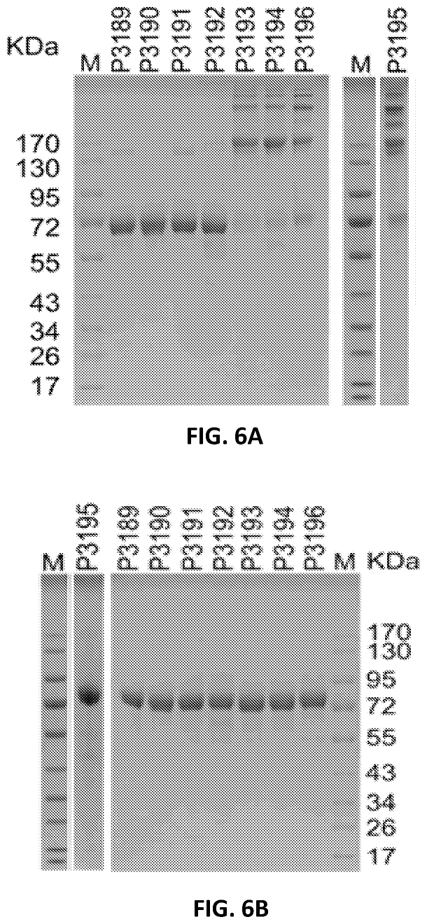

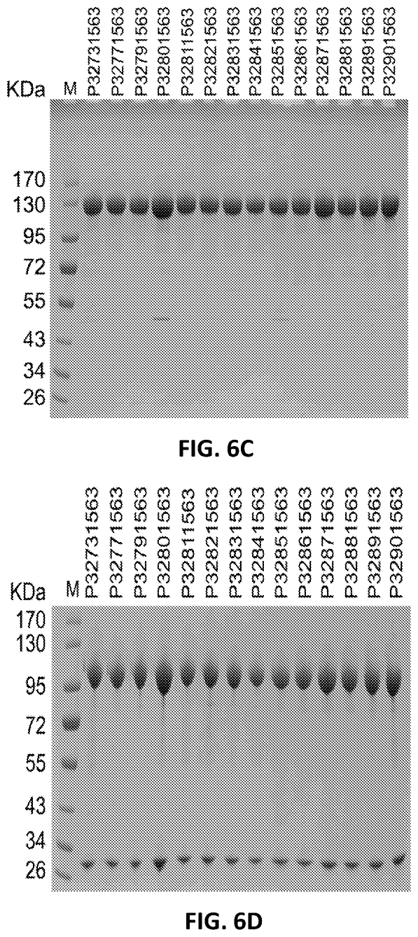

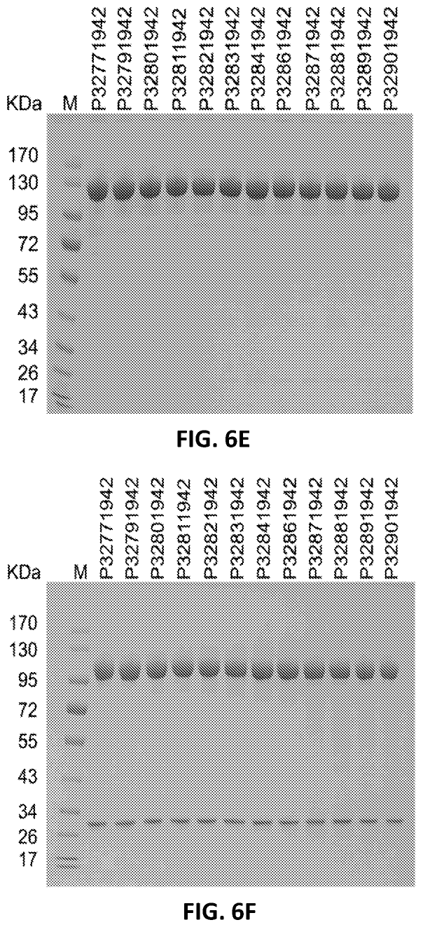

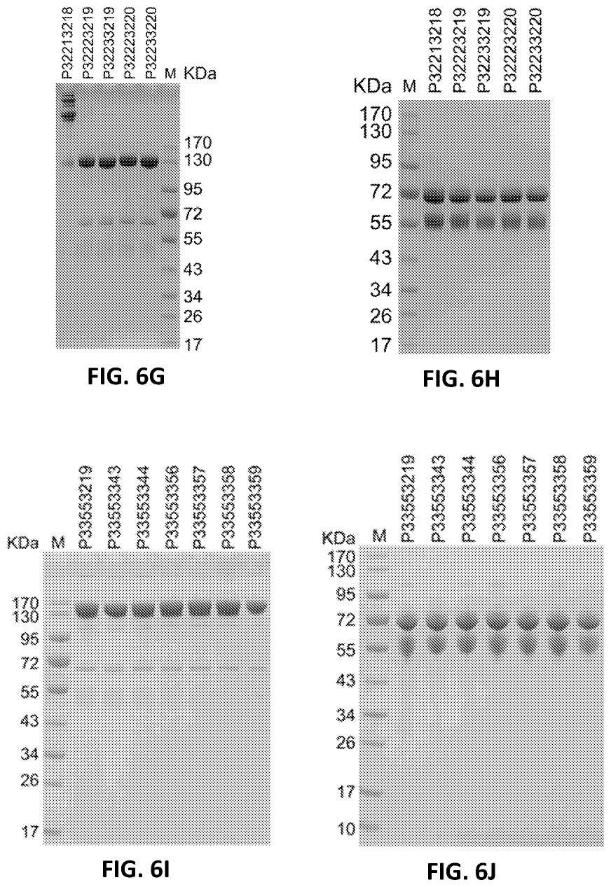

Purified proteins were characterized by SDS-PAGE for purity assessment. Representative SDS-PAGE results of purified proteins are shown in FIGS. 6A-6F. SDS-PAGE gel results of proteins P3189, P3190, P3191, P3192, P3193, P3194, P3195 and P3196 are shown in FIGS. 6A and 6B. Multiple bands are presented on the non-reducing gel (FIG. 6A) for P3194, P3195 and P3196 while one band on the reducing gel (FIG. 6B), indicating abnormal inter-chain di-sulfide bond formation. A higher band between 130 Kd and 170 Kd is observed for P3189 and P3191 (with C74S in p35 and C177A in p40) on a non-reducing gel, indicating inter-chain di-sulfide bond may be formed between C252. One band is present on non-reducing gel for P3190 and P3192.

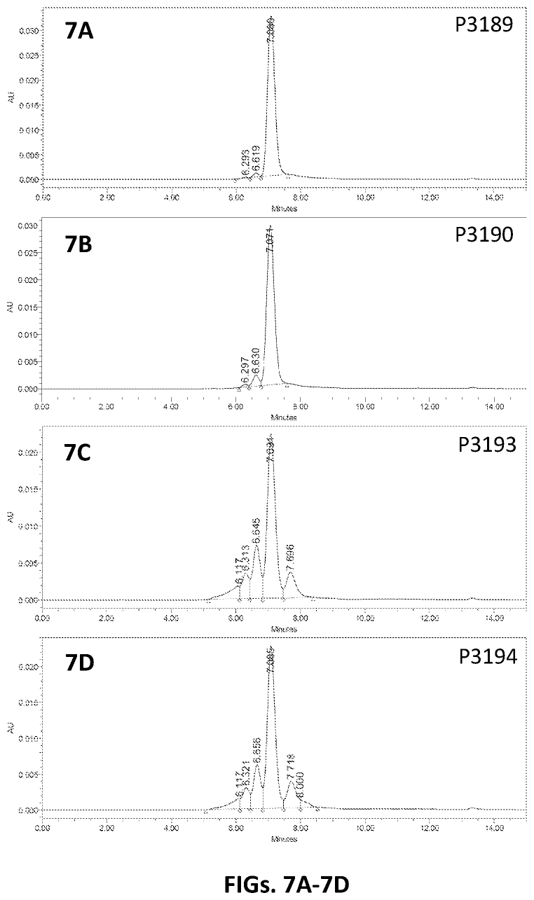

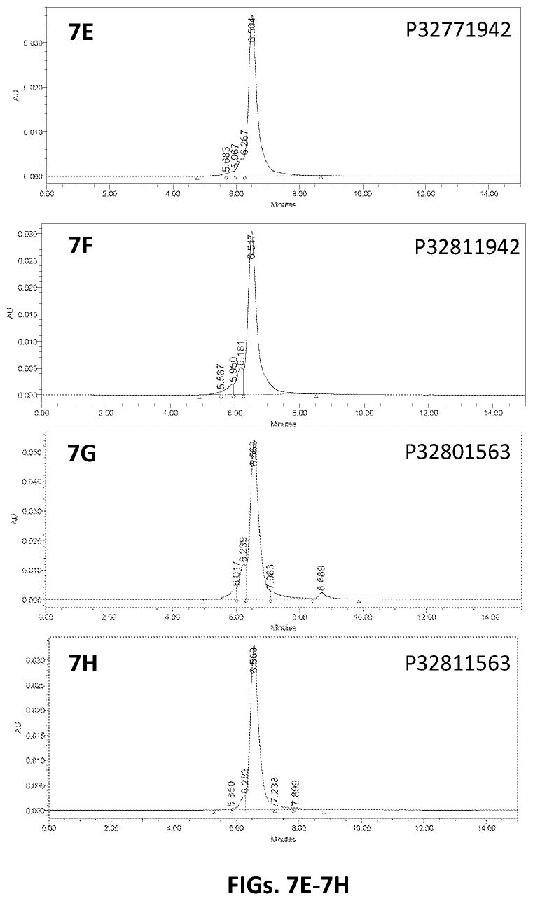

Purified proteins were also characterized by high performance liquid chromatography (HPLC) for homogeneity assessment. HPLC analysis was performed using Zenix-C column (Sepax) and Acquity Arc (Agilent) according to the manufacturer's instructions. Representative HPLC results of purified proteins are shown in FIGS. 7A-7H. HPLC results of P3189, P3190, P3193, and P3194 are shown in FIGS. 7A-7D. P3189 and P3190 purified from CH1 column showed a major monomeric peak with a small fraction of aggregation. P3193 and P3194 purified from CH1 column showed much higher percentage of aggregation. These HPLC results are consistent with SDS-PAGE results.

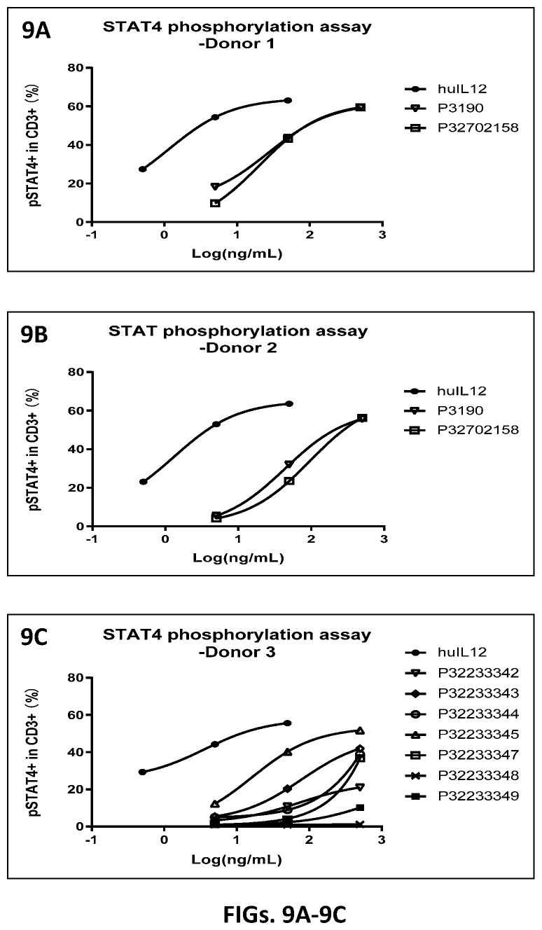

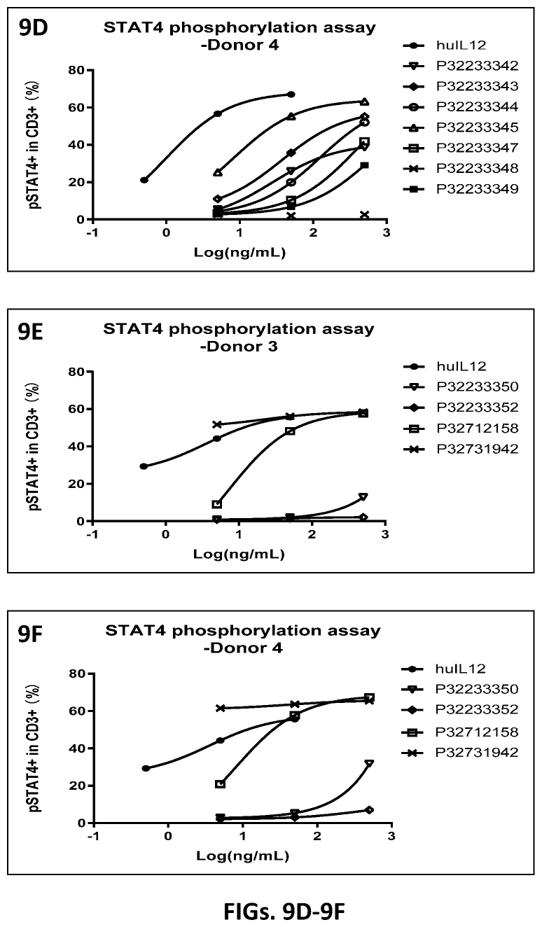

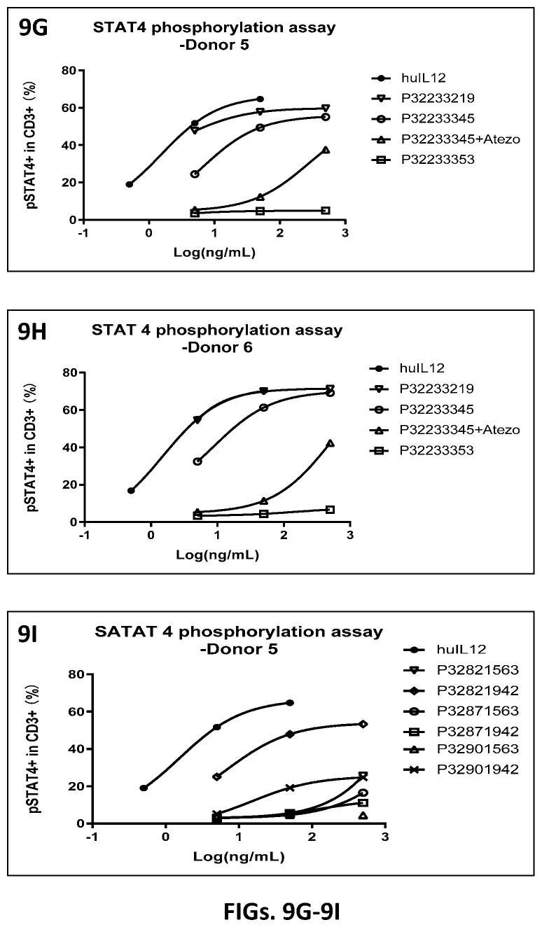

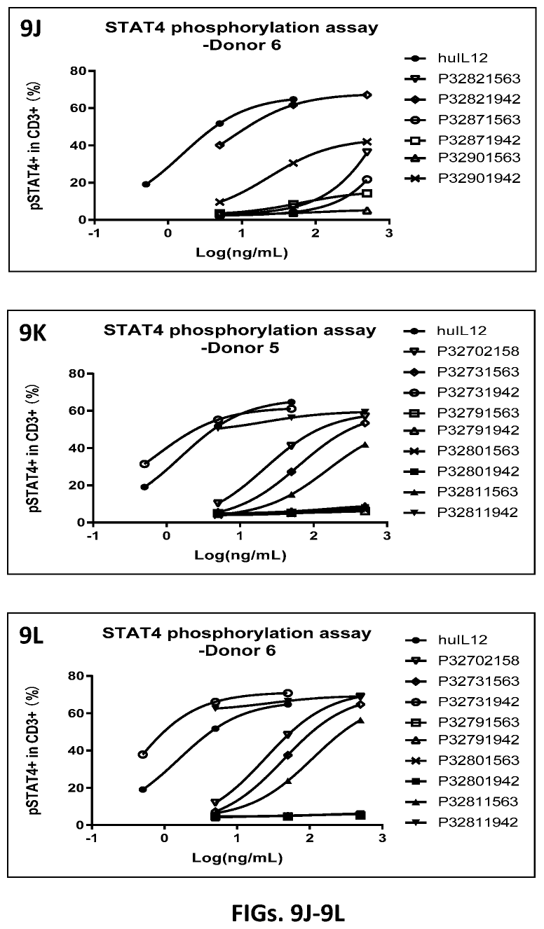

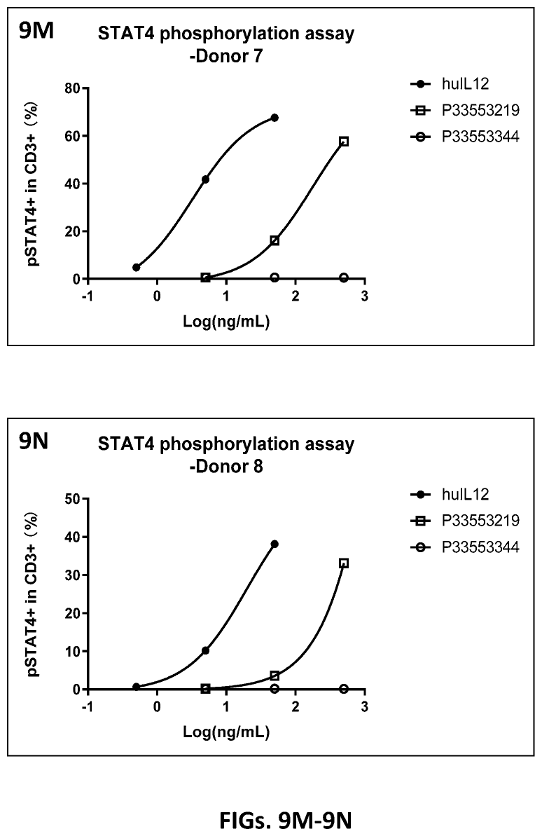

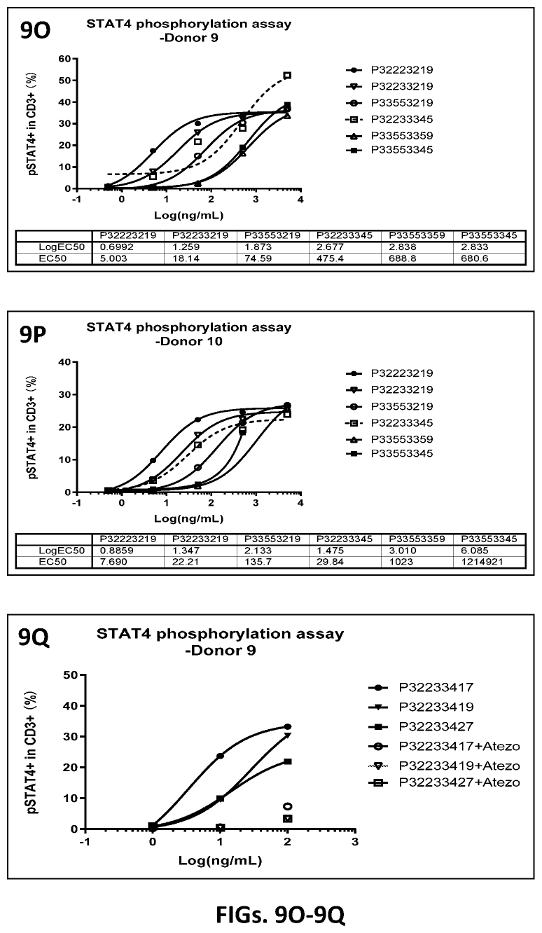

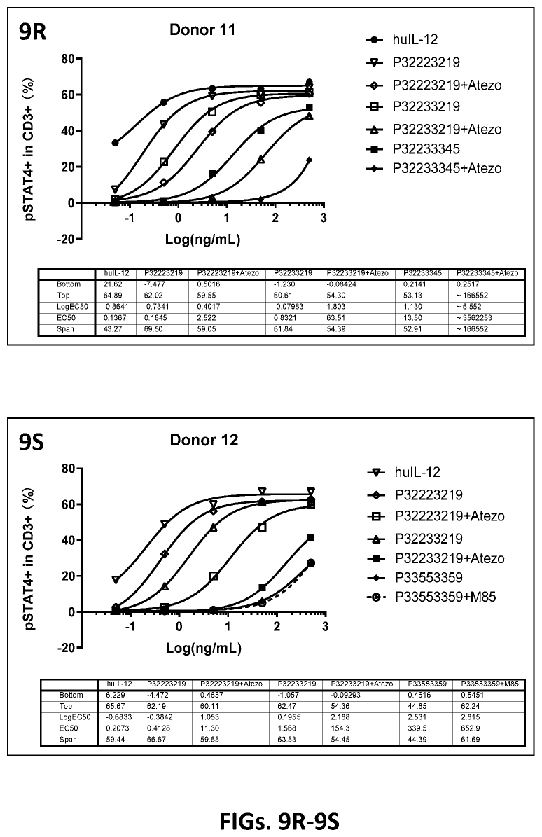

Functional assay—STAT4 phosphorylation. Human PBMCs were isolated from whole blood on a density gradient with Ficoll-Paque PREMIUM 1.084 (GE, 17-5446-02). Freshly isolated human PBMCs were activated with 0.5 μg/mL immobilized anti-CD3 antibodies for 3 days. Culture medium was RPMI 1640 medium supplemented with 10% FBS and 10,000 U/mL Penicillin-Streptomycin. After 3d of culture, activated PBMCs were washed with complete culture medium twice to remove cytokines in the supernatant and transferred into 96-well plates (3E5 cells per well). The indicated amounts of IL-12 or IL-12 fusion proteins were applied to activated PBMCs for 20 min at 37° C. to induce STAT4 phosphorylation. Cells were fixed immediately with Cytofix Fixation buffer (BD Bioscience, 554655) to preserve the phosphorylation status for 15 min on ice. The cells were stained with CD3 FITC (BD Bioscience, 561807) for 30 min on ice and permeabilized with Phosflow Perm buffer III (BD Bioscience, 558050) for 30 min on ice. Then the cells were stained with p-STAT4 AF647 (BD Bioscience, 562074) for 60 min on ice. Data were acquired using a Beckman CytoFLEX flow cytometer and were analyzed using Flowjo. The percentage of pSTAT4⁺ population in CD3⁺ T cells was plotted against cytokine concentration. Dose-response curve was fitted with Graphpad Prism. Exemplary results are summarized in FIGS. 9A, 9B, 9E, 9F, 9I, 9J, 9K, and 9L. P3190, P32702158, P32712158, and P32732158 show similar activity in STAT4 phosphorylation assays. P32731942 (AtezoABD-p35-p40; Atezolizumab is an anti-PD-L1 antibody) shows higher activity than human IL-12 control. P32811563, P32821563, and P32901563 show attenuated IL-12 activity, and fusion of these p35 mutants (P32811942, P32821942, and P32901942) to an ABD targeted against PD-L1 can restore IL-12 activity partially or completely.

### Example 2

### Engineering and Testing of Attenuated IL-12 Heterodimers

ABD-p35/ABD-p40 fusion proteins were generated by fusing wild-type or mutant p35 to the C-terminus of an LC or Fd region, and fusing wild-type or mutant p40 to the C-terminus of a corresponding Fd or LC (see FIGS. 5B-5E). The combined immunoglobulin ABD specifically binds to PD-L1. Illustrative proteins of the LC-p35/Fd-p40 formats are P32213218, P32223219, P32233219, P32223220, and P32233220. Illustrative proteins of the Fd-p35/LC-p40 format are P32243227, P32253228, P32253229, P32263228, and P32263229. Table E6 shows the p35 and p40 proteins used for each construct.

TABLE E6

| | p35 | | | p40 | | | |
|---|---|---|---|---|---|---|---|
| — | ABD | Mutants | | ABD | Mutants | | |
| P32213218 | Anti-PD-L1-LC | — | — | Anti-PD-L1-Fd | — | — | C252S |
| P32223219 | Anti-PD-L1-LC | C74S | — | Anti-PD-L1-Fd | — | C177A | C252S |
| P32233219 | Anti-PD-L1-LC | C74S | — | Anti PD-L1-Fd | QDQD (258-261) | C177A | C252S |
| P32223220 | Anti-PD-L1-LC | C74S | Extra A198 | Anti-PD-L1-Fd | — | C177A | C252S |
| P32233220 | Anti-PD-L1-LC | C74S | Extra A198 | Anti-PD-L1-Fd | QDQD (258-261) | C177A | C252S |
| P32243227 | Anti-PD-L1-Fd | — | — | antiPD-L1-LC | — | — | C252S |
| P32253228 | Anti-PD-L1-Fd | C74S | — | Anti-PD-L1-LC | — | C177A | C252S |
| P32253229 | Anti-PD-L1-Fd | C74S | — | Anti-PD-L1-LC | QDQD (258-261) | C177A | C252S |
| P32263228 | Anti-PD-L1-Fd | C74S | Extra A198 | Anti-PD-L1-LC | — | C177A | C252S |
| P32263229 | Anti-PD-L1-Fd | C74S | Extra A198 | Anti-PD-L1-LC | QDQD (258-261) | C177A | C252S |

To generate ABD-p35/ABD-p40 fusion proteins with attenuated IL-12R binding activities, point mutations were introduced at position 38, 39, 41, 128, 166, or 167 of p35, based on the construct P32233219 from Table E6 (supra). The design of the attenuated constructs is shown in Table E7 and Table E8.

TABLE E7

| Protein | Position | Protein | Position | Protein | Position |
|---|---|---|---|---|---|
| No. | E38 | No. | F39 | No. | P41 |
| P32233395 | A | P32233413 | A | P32233431 | A |
| P32233396 | D | P32233414 | D | P32233432 | D |
| P32233397 | F | P32233415 | E | P32233433 | E |
| P32233398 | G | P32233416 | G | P32233434 | F |
| P32233399 | H | P32233417 | H | P32233435 | G |
| P32233400 | I | P32233418 | I | P32233436 | H |
| P32233401 | K | P32233419 | K | P32233437 | I |
| P32233402 | L | P32233420 | L | P32233438 | K |
| P32233403 | M | P32233421 | M | P32233439 | L |
| P32233404 | N | P32233422 | N | P32233440 | M |
| P32233405 | P | P32233423 | P | P32233441 | N |
| P32233406 | Q | P32233424 | Q | P32233442 | Q |
| P32233407 | R | P32233425 | R | P32233443 | R |
| P32233408 | S | P32233426 | S | P32233444 | S |
| P32233409 | T | P32233427 | T | P32233445 | T |
| P32233410 | V | P32233428 | V | P32233446 | V |
| P32233411 | W | P32233429 | W | P32233447 | W |
| P32233412 | Y | P32233430 | Y | P32233448 | Y |

TABLE E8

| Protein | Position | Protein | Position | Protein | Position |
|---|---|---|---|---|---|
| No. | K128 | No. | F166 | No. | Y167 |
| P32233449 | A | P32233467 | A | P32233342 | A |
| P32233450 | D | P32233468 | D | P32233343 | D |
| P32233451 | E | P32233469 | E | P32233344 | E |
| P32233452 | F | P32233470 | G | P32233345 | F |
| P32233453 | G | P32233471 | H | P32233346 | G |
| P32233454 | H | P32233472 | I | P32233347 | H |
| P32233455 | I | P32233473 | K | P32233348 | I |
| P32233456 | L | P32233474 | L | P32233502 | K |
| P32233457 | M | P32233475 | M | P32233349 | L |
| P32233458 | N | P32233476 | N | P32233503 | M |
| P32233459 | P | P32233477 | P | P32233350 | N |
| P32233460 | Q | P32233478 | Q | P32233504 | P |
| P32233461 | R | P32233479 | R | P32233351 | Q |
| P32233462 | S | P32233480 | S | P32233505 | R |
| P32233463 | T | P32233481 | T | P32233352 | S |
| P32233464 | V | P32233482 | V | P32233353 | T |
| P32233465 | W | P32233483 | W | P32233354 | V |
| P32233466 | Y | P32233484 | Y | P32233506 | W |

Additional fusion proteins were also generated by fusing p35mut and p40mut to the C-terminus of M85 ABDs (anti-FAPa and B7H-3), nivolumab ABDs (anti-PD-1), muJi110 ABDs (anti-PD-1), or non-targeting control ABDs. The design of these fusion proteins is shown in Table E9, Table E10, Table E11, and Table E12.

TABLE E9

| | p35 | | | p40 | | | |
|---|---|---|---|---|---|---|---|
| Protein No. | ABD | Mutants | | ABD | Mutants | | |
| P33553356 | Anti-FAPa/B7H3-LC | C74S | Y167A | Anti-FAPa/B7H3-Fd | QDQD (258-261) | C177A | C252S |
| P33553357 | Anti-FAPa/B7H3-LC | C74S | Y167D | Anti-FAPa/B7H3-Fd | QDQD (258-261) | C177A | C252S |
| P33553358 | Anti-FAPa/B7H3-LC | C74S | Y167E | Anti-FAPa/B7H3-Fd | QDQD (258-261) | C177A | C252S |
| P33553359 | Anti-FAPa/B7H3-LC | C74S | Y167F | Anti-FAPa/B7H3-Fd | QDQD (258-261) | C177A | C252S |
| P33553360 | Anti-FAPa/B7H3-LC | C74S | Y167G | Anti-FAPa/B7H3-Fd | QDQD (258-261) | C177A | C252S |
| P33553361 | Anti-FAPa/B7H3-LC | C74S | Y167H | Anti-FAPa/B7H3-Fd | QDQD (258-261) | C177A | C252S |
| P33553362 | Anti-FAPa/B7H3-LC | C74S | Y167I | Anti-FAPa/B7H3-Fd | QDQD (258-261) | C177A | C252S |
| P33553363 | Anti-FAPa/B7H3-LC | C74S | Y167L | Anti-FAPa/B7H3-Fd | QDQD (258-261) | C177A | C252S |
| P33553364 | Anti-FAPa/B7H3-LC | C74S | Y167N | Anti-FAPa/B7H3-Fd | QDQD (258-261) | C177A | C252S |
| P33553365 | Anti-FAPa/B7H3-LC | C74S | Y167Q | Anti-FAPa/B7H3-Fd | QDQD (258-261) | C177A | C252S |
| P33553366 | Anti-FAPa/B7H3-LC | C74S | Y167S | Anti-FAPa/B7H3-Fd | QDQD (258-261) | C177A | C252S |
| P33553367 | Anti-FAPa/B7H3-LC | C74S | Y167T | Anti-FAPa/B7H3-Fd | QDQD (258-261) | C177A | C252S |
| P33553368 | Anti-FAPa/B7H3-LC | C74S | Y167V | Anti-FAPa/B7H3-Fd | QDQD (258-261) | C177A | C252S |
| P35123513 | Anti-PD-1-LC | C74S | Y167D | Anti-PD-1-Fd | QDQD (258-261) | C177A | C252S |
| P35123514 | Anti-PD-1-LC | C74S | Y167E | Anti-PD-1-Fd | QDQD (258-261) | C177A | C252S |
| P35123515 | Anti-PD-1-LC | C74S | Y167F | Anti-PD-1-Fd | QDQD (258-261) | C177A | C252S |
| P35163564 | Anti-PD-1-LC | C74S | Y167F | Anti-PD-1-Fd | QDQD (258-261) | C177A | C252S |
| P35173565 | Anti-PD-1-LC | C74S | Y167F | Anti-PD-1-Fd | QDQD (258-261) | C177A | C252S |
| P35183565 | Anti-PD-1-LC | C74S | Y167F | Anti-PD-1-Fd | QDQD (258-261) | C177A | C252S |
| P35193565 | Anti-PD-1-LC | C74S | Y167F | Anti-PD-1-Fd | QDQD (258-261) | C177A | C252S |

TABLE E10

| | p35 | | | p40 | | | |
|---|---|---|---|---|---|---|---|
| Protein No. | Fusion partner | Mutants | | Fusion partner | Mutants | | |
| P33553219 | Anti-PD-L1-LC | C74S | — | Anti-FAPa/B7H3-Fd | QDQD (258-261) | C177A | C252S |
| P33553342 | Anti-PD-L1-LC | C74S | Y167A | Anti-FAPa/B7H3-Fd | QDQD (258-261) | C177A | C252S |
| P33553343 | Anti-PD-L1-LC | C74S | Y167D | Anti-FAPa/B7H3-Fd | QDQD (258-261) | C177A | C252S |
| P33553344 | Anti-PD-L1-LC | C74S | Y167E | Anti-FAPa/B7H3-Fd | QDQD (258-261) | C177A | C252S |
| P33553345 | Anti-PD-L1-LC | C74S | Y167F | Anti-FAPa/B7H3-Fd | QDQD (258-261) | C177A | C252S |

TABLE E11

| | hu-p35 | | hu-p40 | | | |
|---|---|---|---|---|---|---|
| Protein No. | Fusion partner | Mutants | Fusion partner | Mutants | | |
| P36673219 | Anti-PD-L1-LC | C74S | Anti-PD-L1-Fd | QDQD (258-261) | C177A | C252S |
| P36673668 | Anti-PD-L1-LC | C74S | Anti-PD-L1-Fd | QDQD (258-261) | C177A | C252S |
| P36693670 | Anti-FAPa/B7H3-LC | C74S | Anti-FAPa/B7H3-Fd | QDQD (258-261) | C177A | C252S |
| P36693671 | Anti-FAPa/B7H3-LC | C74S | Anti-FAPa/B7H3-Fd | QDQD (258-261) | C177A | C252S |
| P39043906 | Anti-FAPa/B7H3-LC | — | Anti-FAPa/B7H3-Fd | QDQD (258-261) | — | C252S |
| P39043907 | Anti-FAPa/B7H3-LC | — | ALB8-Anti-FAPa/B7H3-Fd | QDQD (258-261) | — | C252S |
| P39053906 | Anti-FAPa/B7H3-LC | — | Anti-FAPa/B7H3-Fd | — | — | C252S |
| P39053907 | Anti-FAPa/B7H3-LC | — | ALB8-Anti-FAPa/B7H3-Fd | — | — | C252S |
| P37133715 | Anti-B7H3-LC | C74S | Anti-B7H3-Fd | QDQD (258-261) | C177A | C252S |
| P37143715 | Anti-B7H3-LC | C74S | Anti-B7H3-Fd | QDQD (258-261) | C177A | C252S |
| P37403715 | Anti-B7H3-LC | C74S | Anti-B7H3-Fd | — | C177A | C252S |
| P37413715 | Anti-B7H3-LC | C74S | Anti-B7H3-Fd | — | C177A | C252S |
| P38143816 | Anti-B7H3-LC | C74S | Anti-B7H3-Fd | QDQD (258-261) | C177A | C252S |
| P38153816 | Anti-B7H3-LC | C74S | Anti-B7H3-Fd | QDQD (258-261) | C177A | C252S |
| P38173819 | Anti-B7H3-LC | — | Anti-B7H3-Fd | — | — | C252S |
| P38183819 | Anti-B7H3-LC | — | Anti-B7H3-Fd | — | — | C252S |
| P37103712 | Anti-PD-L1-LC | C74S | Anti-PD-L1-Fd | QDQD (258-261) | C177A | C252S |
| P37113712 | Anti-PD-L1-LC | C74S | Anti-PD-L1-Fd | QDQD (258-261) | C177A | C252S |
| P37163717 | Anti-B7H3-LC-G4Fc-hole | C74S | Anti-B7H3-Fd-G4Fc-knob | QDQD (258-261) | C177A | C252S |
| P37423717 | Anti-B7H3-LC-G4Fc-hole | C74S | Anti-B7H3-Fd-G4Fc-knob | — | C177A | C252S |
| P37433717 | Anti-B7H3-LC-G4Fc-hole | C74S | Anti-B7H3-Fd-G4Fc-knob | — | C177A | C252S |
| P37633717 | Anti-B7H3-LC-G4Fc-hole | C74S | Anti-B7H3-Fd-G4Fc-knob | — | C177A | C252S |
| P37643717 | Anti-B7H3-LC-G4Fc-hole | C74S | Anti-B7H3-Fd-G4Fc-knob | — | C177A | C252S |

TABLE E12

| | Hu-p35 | | hu-p40 | | | | |
|---|---|---|---|---|---|---|---|
| Protein No. | Fusion partner | Mutants | Fusion Partner | Mutants | | | C-term |
| P40453671 | Anti-FAPa/B7H3-LC | C74S | Anti-FAPa/B7H3-Fd | QDQD (258-261) | C177A | C252S | HAS |
| P40473715 | Anti-B7H3-LC | C74S | Anti-B7H3-Fd | QDQD (258-261) | C177A | C252S | HAS |
| P40463712 | Anti-PD-L1-LC | C74S | Anti-PD-L1-Fd | QDQD (258-261) | C177A | C252S | HAS |

Production, purification and characterization. ABD-p35/ABD-p40 fusion proteins were produced by transient transfection in Expi293 cells and purified by a one-step purification of CaptureSelect™ IgG-CH1 Affinity Matrix (ThermoFisher).

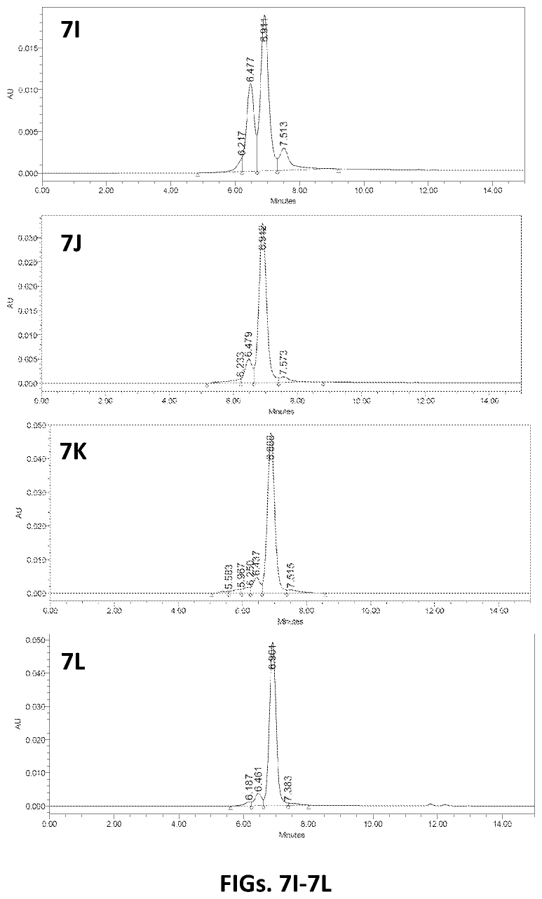

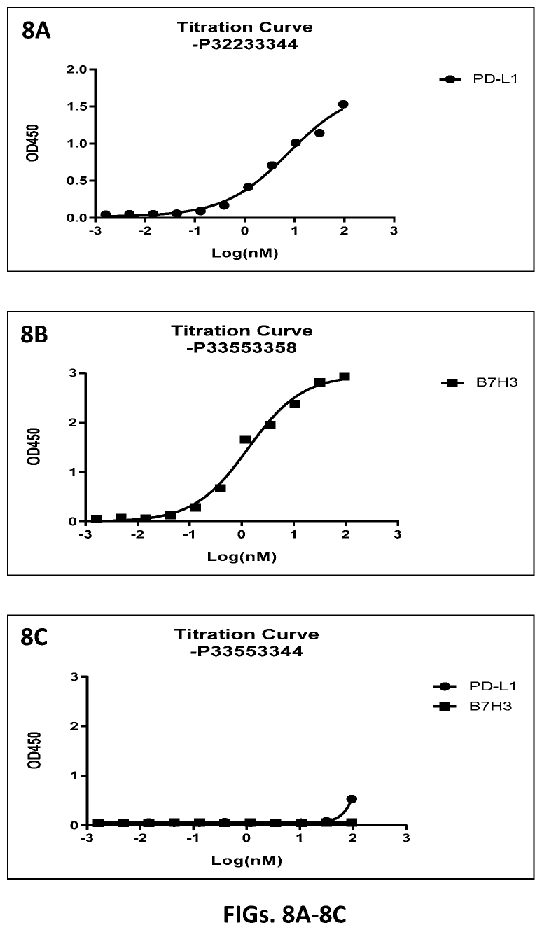

Purified proteins were characterized by SDS-PAGE for purity assessment. Representative SDS-PAGE results are shown in FIGS. 6G-6J. SDS-PAGE results of P32213218, P32223219, P32233219, P32223220, and P32233220 are shown in FIGS. 6G and 6H. Multiple bands are presented on the non-reducing gel (FIG. 6G) for P32213218 while one band on the reducing gel (FIG. 8H), indicating abnormal inter-chain di-sulfide bonds formation.

Purified proteins were also characterized by high performance liquid chromatography (HPLC) for homogeneity assessment. HPLC analysis was performed using Zenix-C column (Sepax) and Acquity Arc (Agilent) according to the manufacturer's instructions. Representative HPLC results were shown in FIGS. 71-7L. P32213218 showed much higher percentage of aggregation compared with P32223219 due to incorrect paring of cysteine residues probably.

Binding assay—ELISA. The binding activity and specificity of ABD-p35/ABD-p40 fusion proteins were determined by ELISA. Microtitre plates were coated with purified PD-L1 or goat anti-mouse Fc antibody (Sigma) overnight at 4° C. The next day, plates were washed with PBS and blocked with 3% non-fat dry milk in PBS. The B7H3-muFc fusion protein was captured by goat anti-mouse Fc antibody. Serially diluted IL-12 fusion proteins were added for binding to the immobilized antigen. Bound antibodies were detected with peroxidase-conjugated anti-human Fab secondary antibody (Jackson Immunoresearch). Exemplary ELISA results are shown in FIGS. 8A-8C. P32233344 (AtezoABd-p35/p40) bound to PD-L1 in ELISA analysis as shown in FIG. 8A. P33553358 (M85ABD-p35/p40) bound to B7H3 as shown in FIG. 8B. P33553344 (M85Fd-p40/AtezoLC-p35) did not bind to either PD-L1 or B7H3 as shown in FIG. 8C.

Functional assay—STAT4 phosphorylation. STAT4 phosphorylation assay was performed as described in Example 1. Exemplary results are shown in FIGS. 9C, 9D, 9E, 9F, 9G, 9H, 9M-9Q, and 9R-9S. P32233219 (AtezoABD-p35/p40) showed higher activity than P33553219 (M85Fd-p40/AtezoLC-p35). Some AtezoABD-p35/p40 with a mutation at Y167E, including P32233342, P32233343, P33233344, P32233345, and P32233347, show attenuated IL-12 activity relative to wild type IL-12 protein. No activity was observed for P33553344 (M85Fd-p40/AtezoLC-p35) with the same Y167 mutation as P32233344 in the current STAT4 phosphorylation assay. Lower activity was observed for P33553359 (M85Fab-p35/p40) and P33553345 (M85Fd-p40/AtezoLC-p35) with the same Y167F mutation as in P32233345. FIG. 9Q shows partial STAT4 assay results of F39 mutants with slightly attenuated IL-12 activity and the activity becomes lower without PDL1 targeting. Blocking with Atezo mAb reduces AtezoABD-p35/p40 activity (FIGS. 9R and 9S). Blocking with M85 does not reduce the activity of M85ABD-p35/p40 (FIG. 9S).

Table E13 below shows the relative activity of exemplary p35 mutants relative to wild-type human IL-12, as measured by phosphorylation of STAT4 in preactivated human PBMCs. % activity of IL-12 is measured by (activity of 100 ng/ml p35 mutants)/(activity of 50 ng/ml IL-12).

TABLE E13

% activity of IL-12

| Protein | Mutant in p35 | Donor A | Donor B |
|---|---|---|---|
| P32233346 | Y167G | 14.70% | 18.72% |
| P32233351 | Y167Q | 7.30% | 5.30% |
| P32233353 | Y167T | 5.02% | 4.48% |
| P32233354 | Y167V | 43.35% | 53.98% |
| P33553342 | Y167A | 5.09% | 3.71% |
| P33553345 | Y167F | 6.05% | 5.96% |
| P33553365 | Y167Q | 10.75% | 10.89% |
| P33553367 | Y167T | 4.98% | 3.14% |
| P33553368 | Y167V | 28.97% | 39.94% |
| P32233395 | E38A | 87.12% | 97.04% |
| P32233396 | E38D | 78.11% | 84.71% |
| P32233397 | E38F | 84.55% | 81.44% |
| P32233398 | E38G | 81.33% | 82.84% |
| P32233399 | E38H | 86.91% | 87.99% |
| P32233400 | E38I | 89.06% | 89.24% |
| P32233401 | E38k | 83.48% | 88.77% |
| P32233402 | E38l | 84.76% | 88.46% |
| P32233403 | E38M | 83.26% | 90.48% |
| P32233404 | E38N | 81.33% | 87.21% |
| P32233405 | E38P | 77.47% | 89.70% |
| P32233406 | E38Q | 90.77% | 92.98% |
| P32233407 | E38R | 75.54% | 84.71% |
| P32233408 | E38S | 86.70% | 91.26% |
| P32233409 | E38T | 89.06% | 92.67% |
| P32233410 | E38V | 87.34% | 92.82% |
| P32233411 | E38W | 80.26% | 87.83% |
| P32233412 | E38Y | 84.76% | 90.64% |
| P32233413 | F39A | 66.95% | 76.29% |
| P32233414 | F39D | 57.08% | 75.20% |
| P32233415 | F39E | 60.94% | 71.76% |
| P32233417 | F39H | 89.27% | 93.92% |
| P32233418 | F39I | 60.94% | 67.71% |
| P32233419 | F39K | 68.45% | 77.07% |
| P32233420 | F39L | 84.12% | 90.64% |
| P32233421 | F39M | 79.83% | 85.65% |
| P32233422 | F39N | 78.11% | 81.12% |
| P32233423 | F39P | 41.63% | 51.33% |
| P32233424 | F39Q | 77.90% | 80.19% |
| P32233425 | F39R | 69.53% | 73.48% |
| P32233426 | F39S | 80.47% | 82.53% |
| P32233427 | F39T | 53.86% | 59.91% |
| P32233428 | F39V | 50.21% | 64.27% |
| P32233429 | F39W | 80.26% | 82.68% |
| P32233430 | F39Y | 95.49% | 88.30% |
| P32233431 | P41A | 83.48% | 84.40% |
| P32233432 | P41D | 78.33% | 76.91% |
| P32233433 | P41E | 48.28% | 57.57% |
| P32233434 | P41F | 39.70% | 45.87% |
| P32233435 | P41G | 83.26% | 81.59% |
| P32233436 | P41H | 62.88% | 65.99% |
| P32233437 | P41I | 67.60% | 60.84% |
| P32233438 | P41K | 71.03% | 75.35% |
| P32233439 | P41L | 57.30% | 54.13% |
| P32233440 | P41M | 70.82% | 75.98% |
| P32233441 | P41N | 56.44% | 66.15% |
| P32233442 | P41Q | 74.68% | 76.44% |
| P32233443 | P41R | 64.38% | 61.93% |
| P32233445 | P41T | 76.39% | 81.59% |
| P32233447 | P41W | 31.33% | 37.91% |
| P32233449 | K128A | 91.20% | 87.36% |
| P32233450 | K128D | 58.80% | 68.49% |
| P32233452 | K128F | 64.16% | 70.36% |
| P32233453 | K128G | 83.91% | 85.96% |
| P32233454 | K128H | 73.18% | 77.69% |
| P32233455 | K128I | 66.31% | 73.17% |
| P32233456 | K128L | 77.25% | 74.73% |
| P32233457 | K128M | 82.40% | 78.78% |
| P32233458 | K128N | 82.19% | 80.66% |
| P32233459 | K128P | 68.67% | 69.73% |
| P32233460 | K128Q | 81.97% | 81.28% |

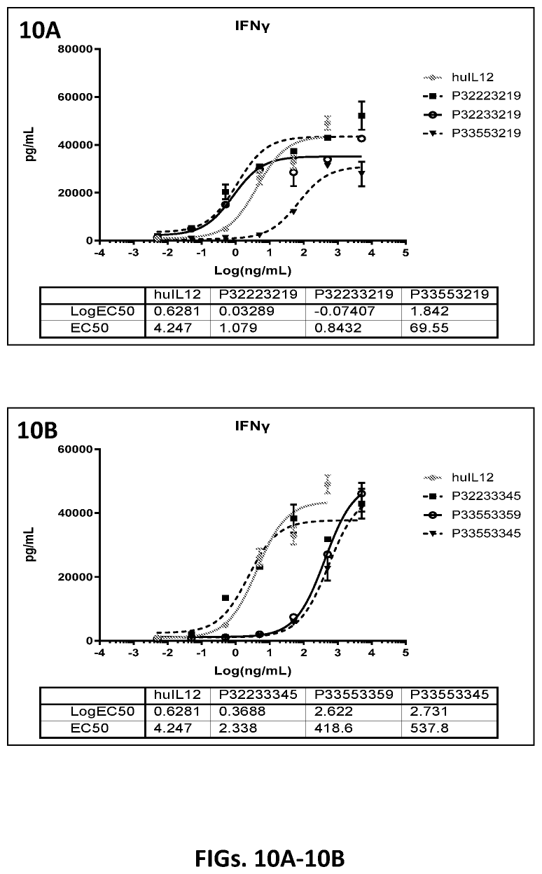

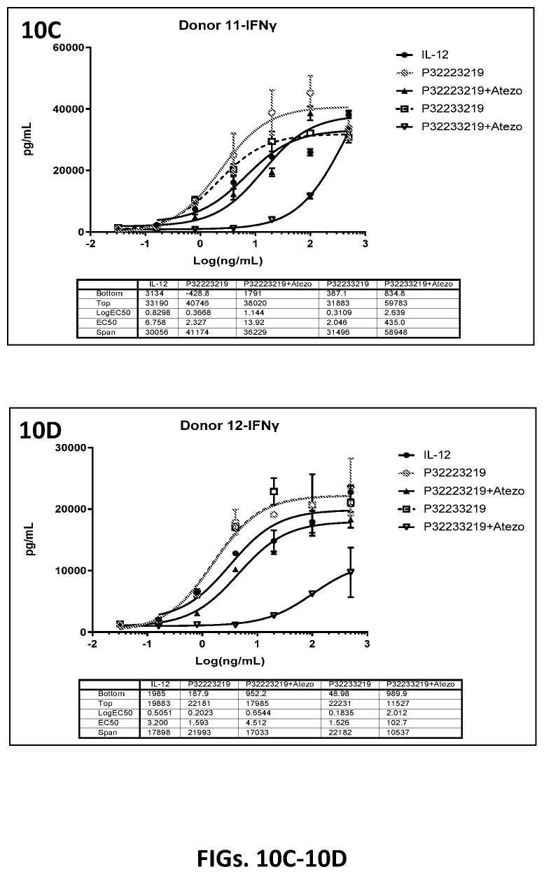

Functional assay—IFNγ production. Human PBMCs were isolated and activated as for the STAT4 phosphorylation assay. Activated PBMCs were seeded into 96-well plate at 2E5 cells/well. The indicated amounts of IL-12 or IL-12 fusion proteins were added to achieve a final concentration from 0-10 μg/mL. Cells in the culture medium alone served as controls. After 48 hrs, IL-12-dependent secretion of IFNγ into the supernatant was quantified via ELISA (Biolegend, 430104). Dose-response curve was fitted with Graphpad Prism. Exemplary results are summarized in FIGS. 10A-10D. P32223219 and P32233219 show similar activity in the IFNγ assay and slightly higher activity than huIL-12. P33553219 shows much lower activity without PDL1 targeting. P32233345 with Y167F mutation shows slighter lower activity than huIL-12, but P33553359 and P33553345 show much lower activity without PDL1 targeting. Consistent with pSTAT4 phosphorylation assay, blocking with Atezo mAb reduces AtezoABD-p35/p40 activity (FIGS. 10C-10D).

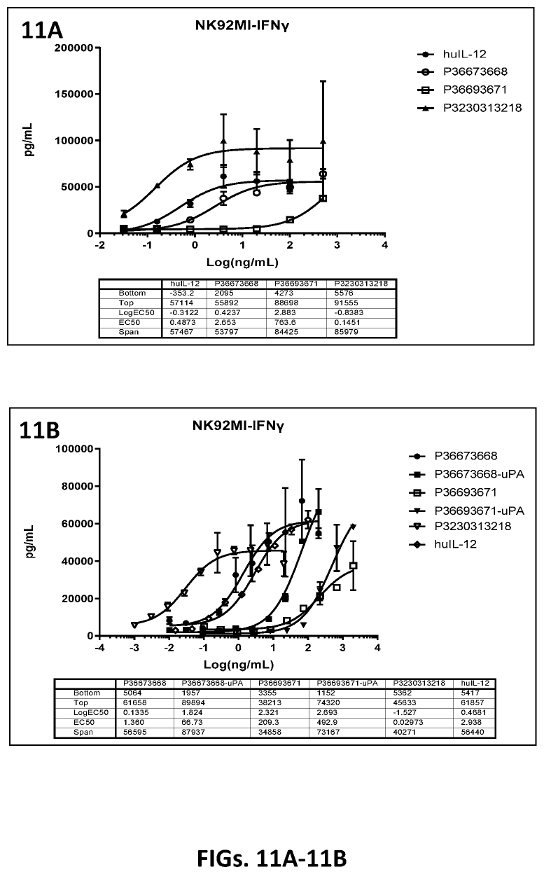

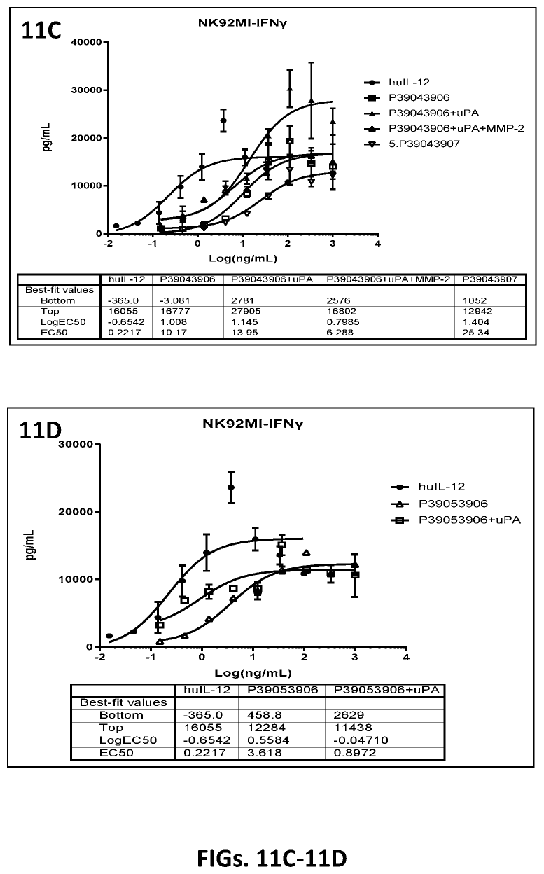

Functional assay—IFNγ production in NK92MI. NK92MI cells were seeded into 96-well plate at 2E4 cells/well. The indicated amounts of IL-12 or IL-12 fusion proteins were added into corresponding wells. Cells in the culture medium alone were served as controls. After 48 hrs, IL-12-dependent secretion of IFN-γ into the supernatant was quantified via ELISA (Biolegend, 430104). Dose-response curve was fitted with Graphpad Prism. Exemplary results are summarized in FIGS. 11A-11D. P36673668 (AtezoABD-p35/p40) shows similar activity as IL-12 while P36693671 (M85ABD-p35/p40) shows about 70 fold lower activity compared with IL-12 (FIGS. 11A and 11B). Protease cleavage between Fd and p40 does not improve IL-12 activity. P39043906 and P39053906 (M85ABD-p35/p40) with di-sulfide bond between p35 and p40 show higher activity compared with P36693671.

1. An attenuated protein homodimer, comprising a first polypeptide and a second polypeptide, wherein:
the first and the second polypeptide comprise, in an N- to C-terminal orientation, a region of an immunoglobulin antigen binding domain (ABD), an IL-12A (p35) protein, a linker, and an IL-12B (p40) protein,
wherein the ABD specifically binds to a cell surface protein expressed on a cell, a plasma protein, or an extracellular matrix (ECM) protein, wherein the IL-12A protein of the first polypeptide is bound to the IL-12B protein of the second polypeptide, and wherein the IL-12B protein of the first polypeptide is bound to the IL-12A protein of the second polypeptide, wherein said binding partially masks a binding site of IL-12 protein(s) that otherwise binds to an IL-12Rβ1/IL-12Rβ2 receptor complex on the surface of an immune cell in vitro or in vivo and attenuates or reduces at least one IL-12 signaling activity of the homodimer relative to wild-type IL-12.

2. The attenuated protein homodimer of claim 1, wherein the cell surface protein is inducible and co-expressed on an immune cell with the IL-12Rβ1/IL-12Rβ2 receptor complex, or wherein the plasma protein is selected from albumins, globulins, fibrinogens, and clotting factors, or wherein the ECM protein is selected from collagens, elastins, fibronectin, and laminins.

3. The attenuated protein homodimer of claim 1 or 2, wherein the cell surface protein is selected from Programmed cell death protein 1 (PD-1), Programmed death-ligand 1 (PD-L1), B7H3 (CD276), T cell immunoreceptor with Ig and ITIM domains (TIGIT), T-cell immunoglobulin and mucin-domain containing-3 (TIM-3), NKG2D, 4-1BB (CD137), CD3, CD4, CD8, CD25, CD70.

4. The attenuated protein homodimer of any one of claims 1-3, wherein the ABD comprises a (i) variable heavy chain (VH) region and a CH1 region, and (ii) a variable light chain (VL) region and a CL region, optionally wherein the CH1 region and the CL region are bound together via a disulfide bond.

5. The attenuated protein homodimer of claim 4, wherein the C-terminus of the CH1 region is fused to the N-terminus of the p35 protein, optionally via a linker, and wherein the VL/CL region is a separate polypeptide chain that is bound to the VH/CH1 region via the disulfide bond.

6. The attenuated protein homodimer of claim 4, wherein the C-terminus of the CL region is fused to the N-terminus of the p35 protein, optionally via a linker, and wherein the VH/CH1 region is a separate polypeptide chain that is bound to the VL/CL region via the disulfide bond.

7. The attenuated protein homodimer of any one of claims 1-6, wherein the first and second p35 proteins comprise, consist, or consist essentially of an amino acid sequence that is at least 80, 85, 90, 95, 98, or 100% identical to an amino acid sequence selected from Table S1, optionally wherein the p35 protein is a variant that comprises or retains an amino acid substitution at C74 and/or S197, as defined by the mature p35 sequence, optionally C74A or C74S and/or S197A.

8. The attenuated protein homodimer of any one of claims 1-7, wherein the first and second p40 proteins comprise, consist, or consist essentially of an amino acid sequence that is at least 80, 85, 90, 95, 98, or 100% identical to an amino acid sequence selected from Table S2, optionally wherein the p40 protein is a variant that comprises or retains amino acid substitutions at any one or more of Y144, C177, C252, K258, 5259, K260, R261, and/or D290, including combinations thereof, as defined by the mature p40 sequence, including any one or more of Y144F, C177A, C252S, K258Q, S259D, K260Q, R261D, and/or D290A, optionally a QDQD substitution at residues K258-R261.

9. The attenuated protein homodimer of any one of claims 1-8, wherein the linker is a flexible linker, optionally a stable or non-cleavable linker.

10. The attenuated protein homodimer of any one of claims 1-9, wherein the linker is about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

11. The attenuated protein homodimer any one of claims 1-10, wherein the linker is selected from Table L1 or Table L2.

12. The attenuated protein homodimer any one of claims 1-11, wherein the immune cell is selected from one or more of a T cell, a B cell, a natural killer (NK) cell, a monocyte, and a macrophage.

13. The attenuated protein homodimer of claim 12, wherein the immune cell is an exhausted T cell or an exhausted NK cell.

14. The attenuated protein homodimer of any one of claims 1-13, wherein the first polypeptide and the second polypeptide comprise, consist, or consist essentially of an amino acid sequence that is at least 80, 85, 90, 95, 98, or 100% identical to a sequence selected from Table S3, optionally wherein the attenuated proprotein homodimer comprises chains 3 and 4 selected from Table S3, optionally, wherein the VL/CL region is a separate polypeptide chain that is bound to the VH/CH1 region via the disulfide bond, or wherein the VH/CH1 region is a separate polypeptide chain that is bound to the VL/CL region via the disulfide bond.

15. The attenuated protein homodimer of any one of claims 1-14, wherein binding of the ABD to the cell surface protein increases binding (avidity) of the IL-12 protein(s) in the homodimer to the IL-12Rβ1/IL-12Rβ2 receptor complex on the surface of an immune cell in vitro or in vivo, and thereby increases at least one IL-12 signaling activity of the homodimer.

16. The attenuated protein homodimer of claim 15, wherein binding of the ABD to the cell surface protein increases binding (avidity) of the IL-12 protein(s) in the homodimer to the IL-12Rβ1/IL-12Rβ2 receptor complex by about or at least about 10%, 20%, 30%, 40%, 50%, 60%, 70%, 80%, 90%, 100%, 200%, 300%, 400%, 500%, 1000%, 2000%, 3000%, 4000%, or 5000% or more.

17. The attenuated protein homodimer of claim 15 or 16, wherein binding of the ABD to the cell surface protein increases at least one IL-12 signaling activity of the homodimer by about or at least about 10%, 20%, 30%, 40%, 50%, 60%, 70%, 80%, 90%, 100%, 200%, 300%, 400%, 500%, 1000%, 2000%, 3000%, 4000%, or 5000% or more.

18. The attenuated protein homodimer of any one of claims 1-17, wherein the at least one IL-12 signaling activity is selected from one or more of stimulating growth and function of T cells, enhancing cytotoxic activity of NK cells and/or CD8+ T cells, stimulating production of interferon-γ (IFN-γ) and/or tumor necrosis factor-a (TNF-α), and inhibiting angiogenesis.

19. The attenuated protein homodimer of any one of claims 1-18, which is substantially in homodimeric form in a physiological solution, or under physiological conditions, optionally in vivo conditions.

20. An attenuated protein heterodimer, comprising a first polypeptide and a second polypeptide, wherein:
(a) the first polypeptide comprises, in an N- to C-terminal orientation, a variable heavy chain (VH) region and a CH1 region, an optional linker, and an IL-12A (p35) protein, and the second polypeptide comprises, in an N- to C-terminal orientation, a variable light chain (VL) region and a CL region, an optional linker, and an IL-12B (p40) protein, wherein the p35 protein of the first polypeptide is bound to the p40 protein of the second polypeptide; or
(b) the first polypeptide comprises, in an N- to C-terminal orientation, a variable heavy chain (VH) region and a CH1 region, an optional linker, and an IL-12B (p40) protein, and the second polypeptide comprises, in an N- to C-terminal orientation, a variable light chain (VL) region and a CL region, an optional linker, and an IL-12A (35) protein, wherein the p40 protein of the first polypeptide is bound to the p35 protein of the second polypeptide,
wherein the CH1 region and the CL region of (a) or (b) are bound together via a disulfide bond to form an immunoglobulin antigen binding domain (ABD), wherein the ABD specifically binds to a cell surface protein expressed on a cell, a plasma protein, or an extracellular matrix (ECM) protein, and wherein at least one of the p35 and/or the p40 protein is a variant that has one or more amino acid alterations relative to a wild-type p35 or p40 sequence, and which has reduced binding affinity to a wild-type IL-12Rβ1/IL-12Rβ2 receptor complex on the surface of an immune cell in vitro or in vivo relative to that of the wild-type p35 and/or p40 sequence.

21. The attenuated protein heterodimer of claim 20, wherein the cell surface protein is inducible and co-expressed on the immune cell with the IL-12Rβ1/IL-12Rβ2 receptor complex, or wherein the plasma protein is selected from albumins, globulins, fibrinogens, and clotting factors, or wherein the ECM protein is selected from collagens, elastins, fibronectin, and laminins.

22. The attenuated protein heterodimer of claim 20 or 21, wherein the cell surface protein is selected from Programmed cell death protein 1 (PD-1), Programmed death-ligand 1 (PD-L1), B7H3 (CD276), T cell immunoreceptor with Ig and ITIM domains (TIGIT), T-cell immunoglobulin and mucin-domain containing-3 (TIM-3), NKG2D, 4-1BB (CD137), CD3, CD4, CD8, CD25, and CD70.

23. The attenuated protein heterodimer of any one of claims 20-22, wherein the p35 protein is a variant that has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p35 sequence.

24. The attenuated protein heterodimer of claim 23, wherein the p35 protein has an amino acid substitution at any one or more of Y167, E38, F39, P41, K128, F166, and/or D188, as defined by the mature p35 sequence, optionally selected from any one or more of:
a Y167A, Y167D, Y167E, Y167F, Y167G, Y167H, Y167I, Y167L, Y167N, Y167Q, Y167S, Y167T, or Y167V substitution;
a E38A, E38D, E38F, E38G, E38H, E38I, E38K, E38L, E38M, E38N, E38P, E38Q, E38R, E38S, E38T, E38V, or E38W substitution;
a F39A, F39D, F39E, F39G, F39H, F39I, F39K, F39L, F39M, F39N, F39P, F39Q, F39R, F39S, F39T, F39V, F39W, or F39Y substitution;
a P41A, P41D, P41E, P41F, P41G, P41H, P41I, P41K, P41L, P41M, P41N, P41Q, P41R, P41S, P41T, P41V, P41W, or P41Y substitution;
a K128A, K128D, K128E, K128F, K128G, K128H, K128I, K128L, K128M, K128N, K128P, K128Q, K128R, K128S, K128T, K128V, K128W, or K128Y substitution;
a F166A, F166D, F166E, F166G, F166H, F166I, F166K, F166L, F166M, F166N, F166P, F166Q, F166R, F166S, F166T, F166V, F166W, or F166Y substitution; and
a D188A substitution, including any combination of the foregoing.

25. The attenuated protein heterodimer of any one of claims 20-24, wherein the p35 protein comprises, consists, or consists essentially of an amino acid sequence that is at least 80, 85, 90, 95, 98, or 100% identical to an amino acid sequence selected from Table S1, optionally wherein the p35 protein is a variant that comprises or retains the acid substitution at Y167, and/or comprises or retains an amino acid substitution at C74 and/or S197, as defined by the mature p35 sequence, optionally C74A or C74S and/or S197A.

26. The attenuated protein heterodimer of any one of claims 20-25, wherein the p40 protein is a variant that has reduced binding affinity to wild-type IL-12Rβ1/IL-12Rβ2 receptor complex of about or at least about 2-fold, 5-fold, 10-fold, 50-fold, 100-fold, 1000-fold or more, relative to the binding affinity of the wild-type p40 sequence.

27. The attenuated protein heterodimer of claim 26, wherein the p40 protein has an amino acid substitution at any one or more of D18, E59, K99, and/or K264, as defined by the mature p40 sequence.

28. The attenuated protein heterodimer of any one of claims 20-27, wherein the p40 protein comprises, consists, or consists essentially of an amino acid sequence that is at least 80, 85, 90, 95, 98, or 100% identical to an amino acid sequence selected from Table S2, optionally wherein the p40 protein is a variant that comprises or retains amino acid substitutions at any one or more of Y144, C177, C252, K258, 5259, K260, R261, and/or D290, including combinations thereof, as defined by the mature p40 sequence, including any one or more of Y144F, C177A, C252S, K258Q, S259D, K260Q, R261D, and/or D290A, optionally a QDQD substitution at residues K258-R261.

29. The attenuated protein heterodimer of any one of claims 20-28, wherein the linker is a flexible linker, optionally a stable or non-cleavable linker.

30. The attenuated protein heterodimer any one of claims 20-29, wherein the linker is about 1-50, 1-40, 1-30, 1-20, 1-10, 1-5, 1-4, 1-3 amino acids in length, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acids in length.

31. The attenuated protein heterodimer any one of claims 20-30, wherein the linker is selected from Table L1 or Table L2.

32. The attenuated protein heterodimer of any one of claims 20-31, comprising four polypeptides selected from:
(i) two of the first and second polypeptides of (a); and
(ii) two of the first and second polypeptides of (b),
wherein the four polypeptides are bound together to form an attenuated protein tetramer, optionally wherein the linkers are about or less than about 1, 2, 3, 4, 5, 6, 7, 8, 9, or 10 amino acids in length.

33. The attenuated protein heterodimer any one of claims 20-32, wherein the immune cell is selected from one or more of a T cell, a B cell, a natural killer (NK) cell, a monocyte, and a macrophage.

34. The attenuated protein heterodimer of claim 33, wherein the immune cell is an exhausted T cell or an exhausted NK cell.

35. The attenuated protein heterodimer of any one of claims 20-34, wherein the first polypeptide comprises, consists, or consists essentially of an amino acid sequence that is at least 80, 85, 90, 95, 98, or 100% identical to a sequence selected from Table S4, wherein the second polypeptide comprises, consists, or consists essentially of an amino acid sequence that is at least 80, 85, 90, 95, 98, or 100% identical to a sequence selected from Table S4.

36. The attenuated protein heterodimer of any one of claims 20-35, wherein binding of the ABD to the cell surface protein increases binding (avidity) of the IL-12 protein(s) in the heterodimer to the IL-12Rβ1/IL-12Rβ2 receptor complex on the surface of the immune cell in vitro or in vivo, and thereby increases at least one IL-12 signaling activity of the heterodimer.

37. The attenuated protein heterodimer of claim 36, wherein binding of the ABD to the cell surface protein increases binding (avidity) of the IL-12 protein(s) in the heterodimer to the IL-12Rβ1/IL-12Rβ2 receptor complex by about or at least about 10%, 20%, 30%, 40%, 50%, 60%, 70%, 80%, 90%, 100%, 200%, 300%, 400%, 500%, 1000%, 2000%, 3000%, 4000%, or 5000% or more.

38. The attenuated protein heterodimer of claim 36 or 37, wherein binding of the ABD to the cell surface protein increases at least one IL-12 signaling activity of the heterodimer by about or at least about 10%, 20%, 30%, 40%, 50%, 60%, 70%, 80%, 90%, 100%, 200%, 300%, 400%, 500%, 1000%, 2000%, 3000%, 4000%, or 5000% or more.

39. The attenuated protein heterodimer of any one of claims 20-38, wherein the at least one IL-12 signaling activity is selected from one or more of stimulating growth and function of T cells, enhancing cytotoxic activity of NK cells and/or CD8+ T cells, stimulating production of interferon-γ (IFN-γ) and/or tumor necrosis factor-α (TNF-α), and inhibiting angiogenesis.

40. The attenuated protein heterodimer of any one of claims 20-39, which is substantially in homodimeric form in a physiological solution, or under physiological conditions, optionally in vivo conditions.

41. One or more isolated recombinant nucleic acid molecules encoding the first and second polypeptide of the attenuated protein homodimer of any one of claims 1-19, and optionally the VL/CL region and/or the VH/CH1 region of claim 5 or 6 as a separate polypeptide chain, one or more vectors comprising the recombinant nucleic acid molecules, or one or more host cells comprising the one or more vectors.

42. A method of producing an attenuated protein homodimer of any one of claims 1-19, comprising culturing the one or more host cells of claim 40 under culture conditions suitable for the expression of the attenuated protein homodimer, and isolating the attenuated protein homodimer from the culture.

43. One or more isolated recombinant nucleic acid molecules encoding the first and second polypeptide of the attenuated protein heterodimer of any one of claims 20-40, one or more vectors comprising the recombinant nucleic acid molecules, or one or more host cells comprising the one or more vectors.

44. A method of producing an attenuated protein heterodimer of any one of claims 20-40, comprising
(a) culturing the one or more host cells of claim 43 under culture conditions suitable for the expression of the attenuated protein heterodimer, and isolating the attenuated protein heterodimer from the culture; or
(b) culturing a host cell of claim 43 that expresses the first polypeptide of the heterodimer, culturing a separate host cell of claim 43 that expresses the second polypeptide of the heterodimer, isolating the first and second polypeptides from each separate host cell, and combining the first and second polypeptides to produce the attenuated protein heterodimer.

45. An isolated human IL-12A (p35) protein variant, which comprises, consists, or consists essentially of an amino acid sequence that is at least 80, 85, 90, 95, 98, or 100% identical to the amino acid sequence set forth in SEQ ID NO: 1 or 2, and which has an amino acid substitution at any one or more of E38, F39, P41, C74, K128, F166, Y167, S197, and/or D188, as defined by the mature p35 sequence.

46. The isolated human p35 protein variant of claim 45, which has an amino acid substitution selected from any one or more of:
a Y167A, Y167D, Y167E, Y167F, Y167G, Y167H, Y167I, Y167L, Y167N, Y167Q, Y167S, Y167T, or Y167V substitution;
a E38A, E38D, E38F, E38G, E38H, E38I, E38K, E38L, E38M, E38N, E38P, E38Q, E38R, E38S, E38T, E38V, or E38W substitution;
a F39A, F39D, F39E, F39G, F39H, F39I, F39K, F39L, F39M, F39N, F39P, F39Q, F39R, F39S, F39T, F39V, F39W, or F39Y substitution;
a P41A, P41D, P41E, P41F, P41G, P41H, P41I, P41K, P41L, P41M, P41N, P41Q, P41R, P41S, P41T, P41V, P41W, or P41Y substitution;
a K128A, K128D, K128E, K128F, K128G, K128H, K128I, K128L, K128M, K128N, K128P, K128Q, K128R, K128S, K128T, K128V, K128W, or K128Y substitution;
a F166A, F166D, F166E, F166G, F166H, F166I, F166K, F166L, F166M, F166N, F166P, F166Q, F166R, F166S, F166T, F166V, F166W, or F166Y substitution; and
a D188A substitution, including any combination of the foregoing.

47. The isolated p35 protein variant of claim 45 or 46, which has reduced binding affinity to wild-type IL-12Rβ31/IL-12Rβ2 receptor complex of about or at least about 2-fold, 5-fold, 10-fold, 50-fold, 100-fold, 1000-fold or more, relative to the binding affinity of the wild-type p35 sequence.

48. An isolated recombinant nucleic acid molecule encoding the human IL-12A (p35) protein variant of any one of claims 45-47, a vector comprising the recombinant nucleic acid molecule, a host cell comprising the vector.

49. A method of producing the human IL-12A (p35) protein variant of any one of claims 45-47, comprising culturing the host cell of claim 48 under culture conditions suitable for the expression of the p35 protein variant, and isolating the p35 protein variant from the culture.

45. A pharmaceutical composition, comprising a pharmaceutically acceptable carrier and the attenuated protein homodimer of any one of claims 1-19, or the attenuated protein heterodimer of any one of claims 20-40.

46. A method of treating disease in a subject, and/or a method of enhancing an immune response in a subject, comprising administering to the subject a therapeutically effective amount of the pharmaceutical composition of claim 45.

47. The method of claim 46, wherein the disease is selected from one or more of a cancer, a viral infection, and an immune disorder.

48. The method of claim 47, wherein the cancer is a primary cancer or a metastatic cancer, and is selected from one or more of melanoma (optionally metastatic melanoma), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

49. The method of any one of claims 45-48, wherein following administration, the attenuated protein homodimer or heterodimer is activated through binding of the ABD to the cell surface receptor on an immune cell in vivo, optionally in a tumor microenvironment, which increases binding (avidity) of the IL-12 protein(s) to the IL-12Rβ1/IL-12Rβ2 receptor complex on the surface of the immune cell, and thereby increases at least one IL-12 signaling activity.

50. The method of claim 49, wherein the immune cell is selected from one or more of a T cell, a B cell, a natural killer cell, a monocyte, and a macrophage.

51. The method of claim 50, wherein the immune cell is an exhausted T cell or an exhausted NK cell.

52. The method of any one of claims 45-51, wherein administration of the attenuated protein homodimer or heterodimer increases an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the immune response is an anti-cancer or anti-viral immune response.

53. The method of any one of claims 45-52, wherein administration of the attenuated protein homodimer or heterodimer increases cell-killing in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, relative to a control, optionally wherein the cell-killing is cancer cell-killing or virally-infected cell-killing.

54. The method of any one of claims 46-53, wherein the viral infection is selected from one or more of human immunodeficiency virus (HIV), Hepatitis A, Hepatitis B, Hepatitis C, Hepatitis E, Caliciviruses associated diarrhoea, Rotavirus diarrhoea, Haemophilus influenzae B pneumonia and invasive disease, influenza, measles, mumps, rubella, Parainfluenza associated pneumonia, Respiratory syncytial virus (RSV) pneumonia, Severe Acute Respiratory Syndrome (SARS), Human papillomavirus, Herpes simplex type 2 genital ulcers, Dengue Fever, Japanese encephalitis, Tick-borne encephalitis, West-Nile virus associated disease, Yellow Fever, Epstein-Barr virus, Lassa fever, Crimean-Congo haemorrhagic fever, Ebola haemorrhagic fever, Marburg haemorrhagic fever, Rabies, Rift Valley fever, Smallpox, upper and lower respiratory infections, and poliomyelitis, optionally wherein the subject is HIV-positive.

55. The method of any one of claims 46-54, wherein the immune disorder is selected from one or more of type 1 diabetes, vasculitis, and an immunodeficiency.

56. The method of any one of claims 45-55, wherein the pharmaceutical composition is administered to the subject by parenteral administration.

57. The method of claim 56, wherein the parenteral administration is intravenous administration.

58. Use of a pharmaceutical composition of claim 45 in the preparation of a medicament for treating a disease in a subject, and/or for enhancing an immune response in a subject.

59. A pharmaceutical composition of claim 45 for use in treating a disease in a subject, and/or for enhancing an immune response in a subject.
